SEQUENCE LISTING (149 sequences)

SEQ ID NO: 1 - protein, 1125 aa, Unknown
  Description of Unknown CasY sequence
MRKKLFKGYILHNKRLVYTGKAAIRSIKYPLVAPNKTALNNLSEKIIYDYEHLFGPLNVASYARNSNRYSLVDFWIDSLRAGVIWQSKSTSLIDLISKLEGSKSPSEKIFEQIDFELKNKLDKEQFKDIILLNTGIRSSSNVRSLRGRFLKCFKEEFRDTEEVIACVDKWSKDLIVEGKSILVSKQFLYWEEEFGIKIFPHFKDNHDLPKLTFFVEPSLEFSPHLPLANCLERLKKFDISRESLLGLDNNFSAFSNYFNELFNLLSRGEIKKIVTAVLAVSKSWENEPELEKRLHFLSEKAKLLGYPKLTSSWADYRMIIGGKIKSWHSNYTEQLIKVREDLKKHQIALDKLQEDLKKVVDSSLREQIEAQREALLPLLDTMLKEKDFSDDLELYRFILSDFKSLLNGSYQRYIQTEEERKEDRDVTKKYKDLYSNLRNIPRFFGESKKEQFNKFINKSLPTIDVGLKILEDIRNALETVSVRKPPSITEEYVTKQLEKLSRKYKINAFNSNRFKQITEQVLRKYNNGELPKISEVFYRYPRESHVAIRILPVKISNPRKDISYLLDKYQISPDWKNSNPGEVVDLIEIYKLTLGWLLSCNKDFSMDFSSYDLKLFPEAASLIKNFGSCLSGYYLSKMIFNCITSEIKGMITLYTRDKFVVRYVTQMIGSNQKFPLLCLVGEKQTKNFSRNWGVLIEEKGDLGEEKNQEKCLIFKDKTDFAKAKEVEIFKNNIWRIRTSKYQIQFLNRLFKKTKEWDLMNLVLSEPSLVLEEEWGVSWDKDKLLPLLKKEKSCEERLYYSLPLNLVPATDYKEQSAEIEQRNTYLGLDVGEFGVAYAVVRIVRDRIELLSWGFLKDPALRKIRERVQDMKKKQVMAVFSSSSTAVARVREMAIHSLRNQIHSIALAYKAKIIYEISISNFETGGNRMAKIYRSIKVSDVYRESGADTLVSEMIWGKKNKQMGNHISSYATSYTCCNCARTPFELVIDNDKEYEKGGDEFIFNVGDEKKVRGFLQKSLLGKTIKGKEVLKSIKEYARPPIREVLLEGEDVEQLLKRRGNSYIYRCPFCGYKTDADIQAALNIACRGYISDNAKDAVKEGERKLDYILEVRKLWEKNGAVLRSAKFL

SEQ ID NO: 2 - protein, 1226 aa, Unknown
  Description of Unknown CasY sequence
MQKVRKTLSEVHKNPYGTKVRNAKTGYSLQIERLSYTGKEGMRSFKIPLENKNKEVFDEFVKKIRNDYISQVGLLNLSDWYEHYQEKQEHYSLADFWLDSLRAGVIFAHKETEIKNLISKIRGDKSIVDKFNASIKKKHADLYALVDIKALYDFLTSDARRGLKTEEEFFNSKRNTLFPKFRKKDNKAVDLWVKKFIGLDNKDKLNFTKKFIGFDPNPQIKYDHTFFFHQDINFDLERITTPKELISTYKKFLGKNKDLYGSDETTEDQLKMVLGFHNNHGAFSKYFNASLEAFRGRDNSLVEQIINNSPYWNSHRKELEKRIIFLQVQSKKIKETELGKPHEYLASFGGKFESWVSNYLRQEEEVKRQLFGYEENKKGQKKFIVGNKQELDKIIRGTDEYEIKAISKETIGLTQKCLKLLEQLKDSVDDYTLSLYRQLIVELRIRLNVEFQETYPELIGKSEKDKEKDAKNKRADKRYPQIFKDIKLIPNFLGETKQMVYKKFIRSADILYEGINFIDQIDKQITQNLLPCFKNDKERIEFTEKQFETLRRKYYLMNSSRFHHVIEGIINNRKLIEMKKRENSELKTFSDSKFVLSKLFLKKGKKYENEVYYTFYINPKARDQRRIKIVLDINGNNSVGILQDLVQKLKPKWDDIIKKNDMGELIDAIEIEKVRLGILIALYCEHKFKIKKELLSLDLFASAYQYLELEDDPEELSGTNLGRFLQSLVCSEIKGAINKISRTEYIERYTVQPMNTEKNYPLLINKEGKATWHIAAKDDLSKKKGGGTVAMNQKIGKNFFGKQDYKTVFMLQDKRFDLLTSKYHLQFLSKTLDTGGGSWWKNKNIDLNLSSYSFIFEQKVKVEWDLTNLDHPIKIKPSENSDDRRLFVSIPFVIKPKQTKRKDLQTRVNYMGIDIGEYGLAWTIINIDLKNKKINKISKQGFIYEPLTHKVRDYVATIKDNQVRGTFGMPDTKLARLRENAITSLRNQVHDIAMRYDAKPVYEFEISNFETGSNKVKVIYDSVKRADIGRGQNNTEADNTEVNLVWGKTSKQFGSQIGAYATSYICSFCGYSPYYEFENSKSGDEEGARDNLYQMKKLSRPSLEDFLQGNPVYKTFRDFDKYKNDQRLQKTGDKDGEWKTHRGNTAIYACQKCRHISDADIQASYWIALKQVVRDFYKDKEMDGDLIQGDNKDKRKVNELNRLIGVHKDVPIINKNLITSLDINLL

SEQ ID NO: 3 - protein, 1201 aa, Unknown
  Description of Unknown CasY3 sequence
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAVRGYLKSVNPDRAIKHGDKGLSRDFLCQEEGKLNFEQIGLLI

SEQ ID NO: 4 - protein, 1210 aa, Unknown
  Description of Unknown CasY sequence
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 5 - protein, 1287 aa, Unknown
  Description of Unknown CasY sequence
MKRILNSLKVAALRLLFRGKGSELVKTVKYPLVSPVQGAVEELAEAIRHDNLHLFGQKEIVDLMEKDEGTQVYSVVDFWLDTLRLGMFFSPSANALKITLGKFNSDQVSPFRKVLEQSPFFLAGRLKVEPAERILSVEIRKIGKRENRVENYAADVETCFIGQLSSDEKQSIQKLANDIWDSKDHEEQRMLKADFFAIPLIKDPKAVTEEDPENETAGKQKPLELCVCLVPELYTRGFGSIADFLVQRLTLLRDKMSTDTAEDCLEYVGIEEEKGNGMNSLLGTFLKNLQGDGFEQIFQFMLGSYVGWQGKEDVLRERLDLLAEKVKRLPKPKFAGEWSGHRMFLHGQLKSWSSNFFRLFNETRELLESIKSDIQHATMLISYVEEKGGYHPQLLSQYRKLMEQLPALRTKVLDPEIEMTHMSEAVRSYIMIHKSVAGFLPDLLESLDRDKDREFLLSIFPRIPKIDKKTKEIVAWELPGEPEEGYLFTANNLFRNFLENPKHVPRFMAERIPEDWTRLRSAPVWFDGMVKQWQKVVNQLVESPGALYQFNESFLRQRLQAMLTVYKRDLQTEKFLKLLADVCRPLVDFFGLGGNDIIFKSCQDPRKQWQTVIPLSVPADVYTACEGLAIRLRETLGFEWKNLKGHEREDFLRLHQLLGNLLFWIRDAKLVVKLEDWMNNPCVQEYVEARKAIDLPLEIFGFEVPIFLNGYLFSELRQLELLLRRKSVMTSYSVKTTGSPNRLFQLVYLPLNPSDPEKKNSNNFQERLDTPTGLSRRFLDLTLDAFAGKLLTDPVTQELKTMAGFYDHLFGFKLPCKLAAMSNHPGSSSKMVVLAKPKKGVASNIGFEPIPDPAHPVFRVRSSWPELKYLEGLLYLPEDTPLTIELAETSVSCQSVSSVAFDLKNLTTILGRVGEFRVTADQPFKLTPIIPEKEESFIGKTYLGLDAGERSGVGFAIVTVDGDGYEVQRLGVHEDTQLMALQQVASKSLKEPVFQPLRKGTFRQQERIRKSLRGCYWNFYHALMIKYRAKVVHEESVGSSGLVGQWLRAFQKDLKKADVLPKKGGKNGVDKKKRESSAQDTLWGGAFSKKEEQQIAFEVQAAGSSQFCLKCGWWFQLGMREVNRVQESGVVLDWNRSIVTFLIESSGEKVYGFSPQQLEKGFRPDIETFKKMVRDFMRPPMFDRKGRPAAAYERFVLGRRHRRYRFDKVFEERFGRSALFICPRVGCGNFDHSSEQSAVVLALIGYIADKEGMSGKKLVYVRLAELMAEWKLKKLERSRVEEQSSAQ

SEQ ID NO: 6 - protein, 1192 aa, Unknown
  Description of Unknown CasY sequence
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDSGVAEKIAQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPVGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWGTYWEKRKPEDILGISTQVYWTGGIGESCPAVAVALLGHIRATSTQTEWEKEEVVFGRLKKFFPS

SEQ ID NO: 7 - protein, 1192 aa, Unknown
  Description of Unknown CasY sequence
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDRGVAEKIEQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPAGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWSTYWEKRKPEDILGISTQVYWTGGIGESCPAVAVALLGHIRATSTQTEWEKEEVVFGRLKKFFPS

SEQ ID NO: 8 - protein, 1193 aa, Unknown
  Description of Unknown CasY Ortholog  sequence
MKRIAKFRHDKPVKREAWSKGYRVHKNRIINKVTRSIKYPLVVKDEWKKRLIDDAAHDYRWLVGPINYSDWCRDPNQYSILEFWIDFLCVGGVFQSSHSNICRLAIQLSGGSVFEQEWKDLSPFVRANLIQGIKPAEFIGFLTAEFRSSSNPKNFISKFFEGSNEDLESLTNEFASIVDFIKAKDISLLRKSLPSCKKIAPNLWEKAVGSHSTNELLKLLTKYTRVMLVAEPSHSDRVFSQTVLQSNDQDDPELTGPLPSHKVGKASYLFIPEFIREVNLDKISKLDLSAKSKLAVEQVKKLSELTSDFKQIENQSEAYFGLSTSFNELSNFLGILIRTLRNAPEAILKDQIALCAPLDKDILKITLDWLCDRAQALPENPRFETNWAEYRSYLGGKIKSWFSNYENFFEIPQAASSQQNNNREKKLGNRSAIRALNLKKEAFEKARETFKGDKGTLEKIDLAYRLLGSISPEVLQCDEGLKLYQQFNDELLVLNETINQKFQDAKRDIKAKKEKESFEKLQRNLSSPLPRIPEFFGERAKKGYQKARVSPKLARHLLECLNDWLARFAKVEESAFSEKEFQRILDWLRTSDFLPVFIRKSKDPPSWLRYIARVATGKYYFWVSEYSRKRVQIIDKPIAQNPLKELISWFLLNKDAFSRDNELFKGLSSKMVTLARIMAGILRDRGEGLKELQAMTSKLDNIGLLHPSFSVPVTDSLKDAAFYRAFFSELEGLLNIGRSRLIIERITLQSQQSKNKKTRRPLMPEPFINEDKEVFLAFPKFETKNKVKGTRVVYNSPDEVNWLLSPIRSSKGQLSFMFRCLSEDAKIMTTSGGCSYIVEFKKLLEAQEEVLSIHDCDIIPRAFVSIPFTLERESEETKPDWKPNRFMGVDIGEYAVAYCVIEKGTDSIEILDCGIVRNGAHRVLKEKVDRLKRRQRSMTFGAMDTSIAAARESLVGNYRNRLHAIALKHGAKLVYEYEVSAFESGGNRIKKVYETLKKSDCTGETEADKNARKHIWGETNAVGDQIGAGWTSQTCAKCGRSFGADLKAGNFGVAVPVPEKVEDSKGHYAYHEFPFEDGLKVRGFLKPNKIISDQKELAKAVHAYMRPPLVALGKRKLPKNARYRRGNSSLFRCPFSDCGFTADADIQAAYNIAVKQLYKPKKGYPKERKWQDFVILKPKEPSKLFDKQFYRPN

SEQ ID NO: 9 - protein, 1154 aa, Unknown
  Description of Unknown CasY10 sequence
MKDSKINAPININANNVSKNKTPKKKPRRKSGKRGYRLHDERIAYSGGTGSCRSIKYELLNPDATRKNLLRGSGLQHEIISAVRQDNLLLYGPLNFNDYIFDKDAPNLLHFWTLALSLGFVFSNQNSIEREFKDYLGVSTEEAVLFGKLNETLKAVFDEAKFISGFLYRNFRGLASKTREQRIKLLTDTLREPLDGVNGDSVSEIIKPYAEKWAEYDGECDQFVFKCELFSIKSTDKPRENTRLSFAIDPAFEVMKLDDKTVFFDDLITHYKENCSDEAQAKRFLGIGDNGNYFNGIFGGLFELLTDGDEKICETTDHLARIYGFDETKKTEINKRLVRLAEYARQINRRPCLVKRWSEYRSDFNGTIESWYSNRQSKQNDTLKQLDEKLKLLEEMRASFPTDSDLCGIKSLSETIEFIRSLKGERIARKVTDELESYLAVLGSELNQYTQQNKDHALPLGWQKKLSKHIQSSPLFFGENKIALWEKLINLKELIKTEVKELEVVLAEDFDDYEITDKQVDNLAALAGRFSESPDGSGHPLVTERLAKIESTLGVDFTHKNNRAKFYLSGFERGKFGKLDVPNKIKVSHLFELADLSILYNAVANSPEDGYILRDTAQLSKIILSAKLRDADREKQRKTVLAHSTLQGYSALISKREFVSRYPLQAVNGSQNLMAYDANRKYYYAYNSEKFAGTKELTVALRGNNFGPEAFGGKFKKVPALRVQSSKYQIQFLDWFFEKQKKRKTELGAGGSFTIAEISCKVNWDDKTPVIFEKPDPRLFVSQPFTINPPENSAKKDYARYIGIDIGEYGLAWHLVEVFEDANEDIGGAGKNAVRIKSVEKGFFTDPQQISLKEDVKKLRENQVRATFTSPDTKIARVRESLIGSYRNLLEDLAVRKDARLCFEYEVSGFESGGARISKVYDSIKRSSVAKKENKAENKQSWGKLFGPEFSFKAIEITAAGTSQYCTKCKRWASLAIKDNNNYQLLEWDNGETGDKRGSDGLLAVTLDGEGKETNRTVRLFPKDGKKAGDTIKGKDLKSAIYRAMRPNMRPSEDGSISLGAGMEAVRRDLMPEQWEKLTLEFGQGKPRGNMAIYVCPYCGHISDADMQAAFNIAVRGYLANRDKEKKVKLGKEYLTDEQSKLTFDPVGILEHTT

SEQ ID NO: 10 - protein, 1266 aa, Unknown
  Description of Unknown CasY15 sequence
MPLMIRNTMNEKKTATQRRNARRRRGERARTKSQELRGYRLHDARIEFSGGLGSMRTVKVELLNPDSSREDPQRGQGLQGKVAKAVFDDYRALYGPMNIEDYLSDPDCPSFLGLWVKAVCLGVIMSRKTATDFGELRGGSKSGQAFDSIPEHLRRQLIKLKWLDWYDKGIRKSSSKASRLKSLTDVFANPKQPDQGVMAAWEQGEKLAESSRDIAALGRREFKDKLFAIPPPTSSVVLDDDVKATKVSRDWQWAVDPQFKLPSTDLDITRALEEVDRQWFERLGNNRGMVQQFFAIGDNGNHLNNGLFGHFFASIRSANLADIVAEMGTAFGFSAEERDIVRQRLETLHEYAQGLPEKPVLASRWAEYRTDMTAKLGSWYSNRTSKGAASITQVWGTINTETGEVKDDGLVRTLENIQSDLPDSCSIKEGILQETLDFIGDRRSSTDRAFTDELELYLATLRSDLNTWCQEQSALWEEKQRQVATPASDEKSKKADNPWAGKGSKTDKWLGALHTRIQSSPLFWGVDKLELWKTLANLKQAIRDEIDKLNEQVEVFGRSAYDEPVGKDADSGEGDRRVDQLSYLSARLGDQAHEEVRQRLDAIALALGVKFSERDDLHRFFVSSRARRRAALLAMPNTITVGKLRELADLTPLWERIKKKPEEPRLLADTVALSKVVNSACASRANPSDQIELTTIHSRLDGYSKNIGHTEFISRATVQSTNGAQNTVALDSLVSPRLFYYNFPNIVESAEPHVSHLEVATRGNLGSFEEFAAKEHRTFDRENPQKDSRNRIDSVNPLAVASSRYQIQFFTWWAGLHRSKETALEVGGSFTIAERQVRLDWSQEKPQAVVSEELRVFVSQPFTIVPDDKKRPATSGTRYIGVAIGEYGLAWSCWEFAPGYWNGSVVNPSKVTCLDYGFLAEPGQRRIVERVKKLRESQATKTFTSPDTYIARLRENVVATYQAQLEALMMAYNAQLVFASEISAFETGGNRVKKIYDAIKRSSVFGRSDAEATDNNQHWGKNGNRSSVKDPDKLRLNEAGQVAARVPWAEPVSAWMTSQTCSACGRVYVRAYRGKNSNEPDSGATGEVRYFDNKQQKILTKTIGADTVWVTDQERKEFERGVYNAMRPNAFMPDGRWTAAGEILEAALKSRGTLDGGRGFAGLHLTSKAQVHEYIEGTGKSHADAHGNSAIFICPYTDCGHIADAALQASYNIALRGFAYAIVRKKHPELFAGSGSSTDGDEGGGKKPQQKQAFIDEIVRAAGRAS

SEQ ID NO: 11 - RNA, 5 nt, Artificial Sequence
  Description of Artificial Sequence Synthetic oligonucleotide
uuuuu

SEQ ID NO: 12 - RNA, 8 nt, Artificial Sequence
  Description of Artificial Sequence Synthetic oligonucleotide
uuuuuuuu

SEQ ID NO: 13 - RNA, 10 nt, Artificial Sequence
  Description of Artificial Sequence Synthetic oligonucleotide
uuuuuuuuuu

SEQ ID NO: 14 - DNA, 10 bp, Artificial Sequence
  Description of Artificial Sequence Synthetic oligonucleotide
  Description of Combined DNA/RNA Molecule Synthetic oligonucleotide
ttttuutttt

SEQ ID NO: 15 - DNA, 6 bp, Artificial Sequence
  Description of Artificial Sequence Synthetic oligonucleotide
  Description of Combined DNA/RNA Molecule Synthetic oligonucleotide
ttuutt

SEQ ID NO: 16 - DNA, 6 bp, Artificial Sequence
  Description of Artificial Sequence Synthetic oligonucleotide
  Description of Combined DNA/RNA Molecule Synthetic oligonucleotide
taaugc

SEQ ID NO: 17 - DNA, 6 bp, Artificial Sequence
  Description of Artificial Sequence Synthetic oligonucleotide
  Description of Combined DNA/RNA Molecule Synthetic oligonucleotide
tauggc

SEQ ID NO: 18 - RNA, 5 nt, Artificial Sequence
  Description of Artificial Sequence Synthetic oligonucleotide
uuuuu

SEQ ID NO: 19 - DNA, 8 bp, Artificial Sequence
  Description of Artificial Sequence Synthetic oligonucleotide
ttattatt

SEQ ID NO: 20 - DNA, 6 bp, Artificial Sequence
  Description of Artificial Sequence Synthetic oligonucleotide
tttttt

SEQ ID NO: 21 - DNA, 8 bp, Artificial Sequence
  Description of Artificial Sequence Synthetic oligonucleotide
tttttttt

SEQ ID NO: 22 - DNA, 10 bp, Artificial Sequence
  Description of Artificial Sequence Synthetic oligonucleotide
tttttttttt

SEQ ID NO: 23 - DNA, 12 bp, Artificial Sequence
  Description of Artificial Sequence Synthetic oligonucleotide
tttttttttttt

SEQ ID NO: 24 - DNA, 14 bp, Artificial Sequence
  Description of Artificial Sequence Synthetic oligonucleotide
tttttttttttttt

SEQ ID NO: 25 - DNA, 6 bp, Artificial Sequence
  Description of Artificial Sequence Synthetic oligonucleotide
aaaaaa

SEQ ID NO: 26 - DNA, 6 bp, Artificial Sequence
  Description of Artificial Sequence Synthetic oligonucleotide
cccccc

SEQ ID NO: 27 - DNA, 6 bp, Artificial Sequence
  Description of Artificial Sequence Synthetic oligonucleotide
gggggg

SEQ ID NO: 28 - RNA, 43 nt, Artificial Sequence
  Description of Artificial Sequence Synthetic oligonucleotide
cuccgaauuaucgggaggauaaggccaagacccgcgccgaggu

SEQ ID NO: 29 - RNA, 36 nt, Artificial Sequence
  Description of Artificial Sequence Synthetic oligonucleotide
uuaucgggaggauaaggccaagacccgcgccgaggu

SEQ ID NO: 30 - RNA, 31 nt, Artificial Sequence
  Description of Artificial Sequence Synthetic oligonucleotide
gggaggauaaggccaagacccgcgccgaggu

SEQ ID NO: 31 - RNA, 27 nt, Artificial Sequence
  Description of Artificial Sequence Synthetic oligonucleotide
ggauaaggccaagacccgcgccgaggu

SEQ ID NO: 32 - RNA, 105 nt, Artificial Sequence
  Description of Artificial Sequence Synthetic polynucleotide
cuccgaauuaucgggaggauaaguauggauauuuccacaaucuugaaagaaagauuuguuagccuuuaauccauucuccuuucccuuuauuuuaucugacaacau

SEQ ID NO: 33 - RNA, 50 nt, Artificial Sequence
  Description of Artificial Sequence Synthetic oligonucleotide
auggauauuuccacaaucuugaaagaaagauuuguuagccuuuaauccau

SEQ ID NO: 34 - RNA, 23 nt, Artificial Sequence
  Description of Artificial Sequence Synthetic oligonucleotide
aaggccaagacccgcgccgaggu

SEQ ID NO: 35 - RNA, 24 nt, Artificial Sequence
  Description of Artificial Sequence Synthetic oligonucleotide
uaaggccaagacccgcgccgaggu

SEQ ID NO: 36 - RNA, 25 nt, Artificial Sequence
  Description of Artificial Sequence Synthetic oligonucleotide
auaaggccaagacccgcgccgaggu

SEQ ID NO: 37 - RNA, 26 nt, Artificial Sequence
  Description of Artificial Sequence Synthetic oligonucleotide
gauaaggccaagacccgcgccgaggu

SEQ ID NO: 38 - RNA, 28 nt, Artificial Sequence
  Description of Artificial Sequence Synthetic oligonucleotide
aggauaaggccaagacccgcgccgaggu

SEQ ID NO: 39 - RNA, 29 nt, Artificial Sequence
  Description of Artificial Sequence Synthetic oligonucleotide
gaggauaaggccaagacccgcgccgaggu

SEQ ID NO: 40 - RNA, 30 nt, Artificial Sequence
  Description of Artificial Sequence Synthetic oligonucleotide
ggaggauaaggccaagacccgcgccgaggu

SEQ ID NO: 41 - RNA, 95 nt, Artificial Sequence
  Description of Artificial Sequence Synthetic oligonucleotide
ucgggaggauaaguauggauauuuccacaaucuugaaagaaagauuuguuagccuuuaauccauucuccuuucccuuuauuuuaucugacaacau

SEQ ID NO: 42 - DNA, 200 bp, Artificial Sequence
  Description of Artificial Sequence Synthetic polynucleotide
catgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtaca

SEQ ID NO: 43 - RNA, 26 nt, Artificial Sequence
  Description of Artificial Sequence Synthetic oligonucleotide
gauaaggcaucaauaccaaacucugg

SEQ ID NO: 44 - RNA, 26 nt, Artificial Sequence
  Description of Artificial Sequence Synthetic oligonucleotide
augaaggccaagacccgcgccgaggu

SEQ ID NO: 45 - RNA, 26 nt, Artificial Sequence
  Description of Artificial Sequence Synthetic oligonucleotide
augaaggcaucaauaccaaacucugg

SEQ ID NO: 46 - RNA, 26 nt, Artificial Sequence
  Description of Artificial Sequence Synthetic oligonucleotide
aaaaaggccaagacccgcgccgaggu

SEQ ID NO: 47 - RNA, 26 nt, Artificial Sequence
  Description of Artificial Sequence Synthetic oligonucleotide
aaaaaggcaucaauaccaaacucugg

SEQ ID NO: 48 - RNA, 60 nt, Artificial Sequence
  Description of Artificial Sequence Synthetic oligonucleotide
cuuaguuaaggauguuccagguucuuucgggagccuuggccuucucccuuaaccuaugcc

SEQ ID NO: 49 - RNA, 75 nt, Artificial Sequence
  Description of Artificial Sequence Synthetic oligonucleotide
ugguuccauucuccugagcuccguugagagcgagaaagagaacuagccuucccacucaucacuccggcauauucu

SEQ ID NO: 50 - RNA, 62 nt, Artificial Sequence
  Description of Artificial Sequence Synthetic oligonucleotide
uauuuccacaaucuugaaagaaagauuguuagccuuugauaaggcccccuucuacaguggcc

SEQ ID NO: 51 - RNA, 62 nt, Artificial Sequence
  Description of Artificial Sequence Synthetic oligonucleotide
uauuuccacaaucuugaaagaaagauuguuagccuuugauaaggcuccccuucuacaguggc

SEQ ID NO: 52 - RNA, 62 nt, Artificial Sequence
  Description of Artificial Sequence Synthetic oligonucleotide
uauuuccacaaucuugaaagaaagauuguuagccuuugauaaggcauccccuucuacagugg

SEQ ID NO: 53 - RNA, 62 nt, Artificial Sequence
  Description of Artificial Sequence Synthetic oligonucleotide
uauuuccacaaucuugaaagaaagauuguuagccuuugauaaggccauccccuucuacagug

SEQ ID NO: 54 - RNA, 62 nt, Artificial Sequence
  Description of Artificial Sequence Synthetic oligonucleotide
uauuuccacaaucuugaaagaaagauuguuagccuuugauaaggcucauccccuucuacagu

SEQ ID NO: 55 - RNA, 62 nt, Artificial Sequence
  Description of Artificial Sequence Synthetic oligonucleotide
uauuuccacaaucuugaaagaaagauuguuagccuuugauaaggcuucauccccuucuacag

SEQ ID NO: 56 - RNA, 62 nt, Artificial Sequence
  Description of Artificial Sequence Synthetic oligonucleotide
uauuuccacaaucuugaaagaaagauuguuagccuuugauaaggccuucauccccuucuaca

SEQ ID NO: 57 - RNA, 62 nt, Artificial Sequence
  Description of Artificial Sequence Synthetic oligonucleotide
uauuuccacaaucuugaaagaaagauuguuagccuuugauaaggcgcuucauccccuucuac

SEQ ID NO: 58 - RNA, 62 nt, Artificial Sequence
  Description of Artificial Sequence Synthetic oligonucleotide
uauuuccacaaucuugaaagaaagauuguuagccuuugauaaggcugcuucauccccuucua

SEQ ID NO: 59 - RNA, 62 nt, Artificial Sequence
  Description of Artificial Sequence Synthetic oligonucleotide
uauuuccacaaucuugaaagaaagauuguuagccuuugauaaggccugcuucauccccuucu

SEQ ID NO: 60 - RNA, 62 nt, Artificial Sequence
  Description of Artificial Sequence Synthetic oligonucleotide
uauuuccacaaucuugaaagaaagauuguuagccuuugauaaggcccugcuucauccccuuc

SEQ ID NO: 61 - RNA, 62 nt, Artificial Sequence
  Description of Artificial Sequence Synthetic oligonucleotide
uauuuccacaaucuugaaagaaagauuguuagccuuugauaaggcuccugcuucauccccuu

SEQ ID NO: 62 - RNA, 62 nt, Artificial Sequence
  Description of Artificial Sequence Synthetic oligonucleotide
uauuuccacaaucuugaaagaaagauuguuagccuuugauaaggcuuccugcuucauccccu

SEQ ID NO: 63 - RNA, 62 nt, Artificial Sequence
  Description of Artificial Sequence Synthetic oligonucleotide
uauuuccacaaucuugaaagaaagauuguuagccuuugauaaggcguuccugcuucaucccc

SEQ ID NO: 64 - RNA, 62 nt, Artificial Sequence
  Description of Artificial Sequence Synthetic oligonucleotide
uauuuccacaaucuugaaagaaagauuguuagccuuugauaaggcuguuccugcuucauccc

SEQ ID NO: 65 - RNA, 62 nt, Artificial Sequence
  Description of Artificial Sequence Synthetic oligonucleotide
uauuuccacaaucuugaaagaaagauuguuagccuuugauaaggcauguuccugcuucaucc

SEQ ID NO: 66 - RNA, 62 nt, Artificial Sequence
  Description of Artificial Sequence Synthetic oligonucleotide
uauuuccacaaucuugaaagaaagauuguuagccuuugauaaggcuauguuccugcuucauc

SEQ ID NO: 67 - RNA, 62 nt, Artificial Sequence
  Description of Artificial Sequence Synthetic oligonucleotide
uauuuccacaaucuugaaagaaagauuguuagccuuugauaaggcgaugaagcaggaacaua

SEQ ID NO: 68 - RNA, 62 nt, Artificial Sequence
  Description of Artificial Sequence Synthetic oligonucleotide
uauuuccacaaucuugaaagaaagauuguuagccuuugauaaggcggaugaagcaggaacau

SEQ ID NO: 69 - RNA, 62 nt, Artificial Sequence
  Description of Artificial Sequence Synthetic oligonucleotide
uauuuccacaaucuugaaagaaagauuguuagccuuugauaaggcgggaugaagcaggaaca

SEQ ID NO: 70 - RNA, 62 nt, Artificial Sequence
  Description of Artificial Sequence Synthetic oligonucleotide
uauuuccacaaucuugaaagaaagauuguuagccuuugauaaggcggggaugaagcaggaac

SEQ ID NO: 71 - RNA, 62 nt, Artificial Sequence
  Description of Artificial Sequence Synthetic oligonucleotide
uauuuccacaaucuugaaagaaagauuguuagccuuugauaaggcaggggaugaagcaggaa

SEQ ID NO: 72 - RNA, 62 nt, Artificial Sequence
  Description of Artificial Sequence Synthetic oligonucleotide
uauuuccacaaucuugaaagaaagauuguuagccuuugauaaggcaaggggaugaagcagga

SEQ ID NO: 73 - RNA, 62 nt, Artificial Sequence
  Description of Artificial Sequence Synthetic oligonucleotide
uauuuccacaaucuugaaagaaagauuguuagccuuugauaaggcgaaggggaugaagcagg

SEQ ID NO: 74 - RNA, 62 nt, Artificial Sequence
  Description of Artificial Sequence Synthetic oligonucleotide
uauuuccacaaucuugaaagaaagauuguuagccuuugauaaggcagaaggggaugaagcag

SEQ ID NO: 75 - RNA, 62 nt, Artificial Sequence
  Description of Artificial Sequence Synthetic oligonucleotide
uauuuccacaaucuugaaagaaagauuguuagccuuugauaaggcuagaaggggaugaagca

SEQ ID NO: 76 - RNA, 62 nt, Artificial Sequence
  Description of Artificial Sequence Synthetic oligonucleotide
uauuuccacaaucuugaaagaaagauuguuagccuuugauaaggcguagaaggggaugaagc

SEQ ID NO: 77 - RNA, 62 nt, Artificial Sequence
  Description of Artificial Sequence Synthetic oligonucleotide
uauuuccacaaucuugaaagaaagauuguuagccuuugauaaggcuguagaaggggaugaag

SEQ ID NO: 78 - RNA, 62 nt, Artificial Sequence
  Description of Artificial Sequence Synthetic oligonucleotide
uauuuccacaaucuugaaagaaagauuguuagccuuugauaaggccuguagaaggggaugaa

SEQ ID NO: 79 - RNA, 62 nt, Artificial Sequence
  Description of Artificial Sequence Synthetic oligonucleotide
uauuuccacaaucuugaaagaaagauuguuagccuuugauaaggcacuguagaaggggauga

SEQ ID NO: 80 - RNA, 62 nt, Artificial Sequence
  Description of Artificial Sequence Synthetic oligonucleotide
uauuuccacaaucuugaaagaaagauuguuagccuuugauaaggccacuguagaaggggaug

SEQ ID NO: 81 - RNA, 62 nt, Artificial Sequence
  Description of Artificial Sequence Synthetic oligonucleotide
uauuuccacaaucuugaaagaaagauuguuagccuuugauaaggcccacuguagaaggggau

SEQ ID NO: 82 - RNA, 62 nt, Artificial Sequence
  Description of Artificial Sequence Synthetic oligonucleotide
uauuuccacaaucuugaaagaaagauuguuagccuuugauaaggcgccacuguagaagggga

SEQ ID NO: 83 - RNA, 62 nt, Artificial Sequence
  Description of Artificial Sequence Synthetic oligonucleotide
uauuuccacaaucuugaaagaaagauuguuagccuuugauaaggcggccacuguagaagggg

SEQ ID NO: 84 - RNA, 62 nt, Artificial Sequence
  Description of Artificial Sequence Synthetic oligonucleotide
uauuuccacaaucuugaaagaaagauuguuagccuuugauaaggcgcccuucuacaguggcc

SEQ ID NO: 85 - RNA, 62 nt, Artificial Sequence
  Description of Artificial Sequence Synthetic oligonucleotide
uauuuccacaaucuugaaagaaagauuguuagccuuugauaaggcugcccuucuacaguggc

SEQ ID NO: 86 - RNA, 62 nt, Artificial Sequence
  Description of Artificial Sequence Synthetic oligonucleotide
uauuuccacaaucuugaaagaaagauuguuagccuuugauaaggcaugcccuucuacagugg

SEQ ID NO: 87 - RNA, 62 nt, Artificial Sequence
  Description of Artificial Sequence Synthetic oligonucleotide
uauuuccacaaucuugaaagaaagauuguuagccuuugauaaggccaugcccuucuacagug

SEQ ID NO: 88 - RNA, 62 nt, Artificial Sequence
  Description of Artificial Sequence Synthetic oligonucleotide
uauuuccacaaucuugaaagaaagauuguuagccuuugauaaggcucaugcccuucuacagu

SEQ ID NO: 89 - RNA, 62 nt, Artificial Sequence
  Description of Artificial Sequence Synthetic oligonucleotide
uauuuccacaaucuugaaagaaagauuguuagccuuugauaaggcuucaugcccuucuacag

SEQ ID NO: 90 - RNA, 62 nt, Artificial Sequence
  Description of Artificial Sequence Synthetic oligonucleotide
uauuuccacaaucuugaaagaaagauuguuagccuuugauaaggccuucaugcccuucuaca

SEQ ID NO: 91 - RNA, 62 nt, Artificial Sequence
  Description of Artificial Sequence Synthetic oligonucleotide
uauuuccacaaucuugaaagaaagauuguuagccuuugauaaggcgcuucaugcccuucuac

SEQ ID NO: 92 - RNA, 62 nt, Artificial Sequence
  Description of Artificial Sequence Synthetic oligonucleotide
uauuuccacaaucuugaaagaaagauuguuagccuuugauaaggcugcuucaugcccuucua

SEQ ID NO: 93 - RNA, 62 nt, Artificial Sequence
  Description of Artificial Sequence Synthetic oligonucleotide
uauuuccacaaucuugaaagaaagauuguuagccuuugauaaggccugcuucaugcccuucu

SEQ ID NO: 94 - RNA, 62 nt, Artificial Sequence
  Description of Artificial Sequence Synthetic oligonucleotide
uauuuccacaaucuugaaagaaagauuguuagccuuugauaaggcccugcuucaugcccuuc

SEQ ID NO: 95 - RNA, 62 nt, Artificial Sequence
  Description of Artificial Sequence Synthetic oligonucleotide
uauuuccacaaucuugaaagaaagauuguuagccuuugauaaggcuccugcuucaugcccuu

SEQ ID NO: 96 - RNA, 62 nt, Artificial Sequence
  Description of Artificial Sequence Synthetic oligonucleotide
uauuuccacaaucuugaaagaaagauuguuagccuuugauaaggcuuccugcuucaugcccu

SEQ ID NO: 97 - RNA, 62 nt, Artificial Sequence
  Description of Artificial Sequence Synthetic oligonucleotide
uauuuccacaaucuugaaagaaagauuguuagccuuugauaaggcguuccugcuucaugccc

SEQ ID NO: 98 - RNA, 62 nt, Artificial Sequence
  Description of Artificial Sequence Synthetic oligonucleotide
uauuuccacaaucuugaaagaaagauuguuagccuuugauaaggcuguuccugcuucaugcc

SEQ ID NO: 99 - RNA, 62 nt, Artificial Sequence
  Description of Artificial Sequence Synthetic oligonucleotide
uauuuccacaaucuugaaagaaagauuguuagccuuugauaaggcauguuccugcuucaugc

SEQ ID NO: 100 - RNA, 62 nt, Artificial Sequence
  Description of Artificial Sequence Synthetic oligonucleotide
uauuuccacaaucuugaaagaaagauuguuagccuuugauaaggcuauguuccugcuucaug

SEQ ID NO: 101 - RNA, 62 nt, Artificial Sequence
  Description of Artificial Sequence Synthetic oligonucleotide
uauuuccacaaucuugaaagaaagauuguuagccuuugauaaggccaugaagcaggaacaua

SEQ ID NO: 102 - RNA, 62 nt, Artificial Sequence
  Description of Artificial Sequence Synthetic oligonucleotide
uauuuccacaaucuugaaagaaagauuguuagccuuugauaaggcgcaugaagcaggaacau

SEQ ID NO: 103 - RNA, 62 nt, Artificial Sequence
  Description of Artificial Sequence Synthetic oligonucleotide
uauuuccacaaucuugaaagaaagauuguuagccuuugauaaggcggcaugaagcaggaaca

SEQ ID NO: 104 - RNA, 62 nt, Artificial Sequence
  Description of Artificial Sequence Synthetic oligonucleotide
uauuuccacaaucuugaaagaaagauuguuagccuuugauaaggcgggcaugaagcaggaac

SEQ ID NO: 105 - RNA, 62 nt, Artificial Sequence
  Description of Artificial Sequence Synthetic oligonucleotide
uauuuccacaaucuugaaagaaagauuguuagccuuugauaaggcagggcaugaagcaggaa

SEQ ID NO: 106 - RNA, 62 nt, Artificial Sequence
  Description of Artificial Sequence Synthetic oligonucleotide
uauuuccacaaucuugaaagaaagauuguuagccuuugauaaggcaagggcaugaagcagga

SEQ ID NO: 107 - RNA, 62 nt, Artificial Sequence
  Description of Artificial Sequence Synthetic oligonucleotide
uauuuccacaaucuugaaagaaagauuguuagccuuugauaaggcgaagggcaugaagcagg

SEQ ID NO: 108 - RNA, 62 nt, Artificial Sequence
  Description of Artificial Sequence Synthetic oligonucleotide
uauuuccacaaucuugaaagaaagauuguuagccuuugauaaggcagaagggcaugaagcag

SEQ ID NO: 109 - RNA, 62 nt, Artificial Sequence
  Description of Artificial Sequence Synthetic oligonucleotide
uauuuccacaaucuugaaagaaagauuguuagccuuugauaaggcuagaagggcaugaagca

SEQ ID NO: 110 - RNA, 62 nt, Artificial Sequence
  Description of Artificial Sequence Synthetic oligonucleotide
uauuuccacaaucuugaaagaaagauuguuagccuuugauaaggcguagaagggcaugaagc

SEQ ID NO: 111 - RNA, 62 nt, Artificial Sequence
  Description of Artificial Sequence Synthetic oligonucleotide
uauuuccacaaucuugaaagaaagauuguuagccuuugauaaggcuguagaagggcaugaag

SEQ ID NO: 112 - RNA, 62 nt, Artificial Sequence
  Description of Artificial Sequence Synthetic oligonucleotide
uauuuccacaaucuugaaagaaagauuguuagccuuugauaaggccuguagaagggcaugaa

SEQ ID NO: 113 - RNA, 62 nt, Artificial Sequence
  Description of Artificial Sequence Synthetic oligonucleotide
uauuuccacaaucuugaaagaaagauuguuagccuuugauaaggcacuguagaagggcauga

SEQ ID NO: 114 - RNA, 62 nt, Artificial Sequence
  Description of Artificial Sequence Synthetic oligonucleotide
uauuuccacaaucuugaaagaaagauuguuagccuuugauaaggccacuguagaagggcaug

SEQ ID NO: 115 - RNA, 62 nt, Artificial Sequence
  Description of Artificial Sequence Synthetic oligonucleotide
uauuuccacaaucuugaaagaaagauuguuagccuuugauaaggcccacuguagaagggcau

SEQ ID NO: 116 - RNA, 62 nt, Artificial Sequence
  Description of Artificial Sequence Synthetic oligonucleotide
uauuuccacaaucuugaaagaaagauuguuagccuuugauaaggcgccacuguagaagggca

SEQ ID NO: 117 - RNA, 62 nt, Artificial Sequence
  Description of Artificial Sequence Synthetic oligonucleotide
uauuuccacaaucuugaaagaaagauuguuagccuuugauaaggcggccacuguagaagggc

SEQ ID NO: 118 - protein, 1056 aa, Unknown
  Description of Unknown CasM.21524 sequence
MKGYRLHDQRIAYSGGTGSMRSIKYELVDTDGSEGLRDKVAGAIANDYRTLYGPLNFDDYLAGNRTPSLIDFWLKSLSLGFVFSNQNSIESEFLEYLGKKTIWQNCYECLSDELKGVVDEQAFCQFLIKSHRSVEKKTDEQRQKIILNLVKKGCDTSALLPVAKDWSQKFSLDTDQLQLKCEIFGIPVPIVPQRDLSLSFAVDPNFVVMDCSDRTEFLDQIIKFYEDKVGAAQAKKFLAIGDNGNYFNGLFGNLLTCLKQGEVDSVAEFLDSTYELNNKVEISKRLAELKELADKIGEPELVNKWSDYRSDFNGTIESWYSNRISKQQATLEQLDGKIDKKTGEVTGGLKELLKNISDALPEGNDIKEGILAETIAFLRGHGARIDRKFTDELESYLATLKTDLNEWSQNNKEHKMPTGWQRELSKRVQSSPLFFGENKYALWEQLIRLKGLIRDEVAKLEAVLQGQFEDYAITDKQVDMLAQLAQRIDGDGNPEVIRRLADIERELQVNFGERSERARYFISGFERSKVTQLEIGNRINVSKLAELADLGELYDKLKNAPQDNYVLRDTAQLSKIVVSALVHGSDKEREVVLMHSNLSGYASLISRREFITRCTVQAVNGGQLNLGVRGNKYFYAFLPDKFDARSDVQLFSKTYNFTKADLKDNTSSVPLLAVRSSKYQVQFLDWFMGRHSRKKTELGAGGAFSIAEKTVKLDWSGETPRIAEISDPRVFVSQPFEIKPLGKGTQASDNRFIGVDIGEYGLAWSLIEVNGNNVERLEDGFIADLQQQKLKNAVKRLRESQVRATFGSPDTRVARIRESLIGAYRNQLEDLAMRKNARLSFEYEVSGFEAGGARISKVYDSIKRGDIRKKDNNAANKMAWGDFGVNNWGFETTAAGTSQTCSKCRRWASLAIEDGKSYRLGEYQDKLFKAQIADGEVRLLAKQDTGETVKGKDLKGLIYKAMRPNDDGLGMAIVKRQMDWDKLSKDFGAGKPRGNIAIFVCPYTDCHHIADADLQAALNIAIRGYGKRKSDGKMGKVNDFAEFTKELQYDPVGFAS

SEQ ID NO: 119 - protein, 1338 aa, Unknown
  Description of Unknown CasM.21518 sequence
MNKKSSNSTGYRLHKDRILFSGGEIMRTIKYPLVVEKNNLNSEEIVEKIRQAIINDDRVIRSDINLNDYIEYTKKGNRLYTLIDFWQDCLRAGVIWQPSTSFLLYLINKLYSKPKAIELIENAKPDISRFFDVDKFSKCFILPGEIREGKILKTFKRELIEALKGEFKKGKKEKIKDEDDYLEKFVEKDARKLIREIADCFFSNDILVTHDLKEGKKEYQDRLWEEKFGIKKGKLLENFKLPDHLRNFKNISFFIIPELSDKSKNFDELIELRRKWLLERKICVREDGDYLENEKKLDEELRNLVGLSDNCNPLSNFLGTVFCELLVPNNLNEDNALEKFYDVFTIVEPKIAELNIKDQIMGSLEFLRLRAKQLGSPNLVNFSKSQNLKANESIKLDGWSLYRQNFGSKMQSWFTSYIERNKLLEDSLKNFKEKIKKAQNFIKNLKNISEEPQQEEEAQQEKEEIVELFEKIFSSLEKVNRENFEVFDSLLSSLRKRLNFFYQQYLYNEAKEGDDVKKHKILGPIFKNIEKPIAFYGETQRKKNEKFVEDTIPILEEGTVFLTTLISNLLDSFSPKQVFPDVRKKDETEEIIYRKELQFFWNKLKDLAVNSKEFEKEYQDIIESAVDESELSKLKELFVNKKKNGSKYNKYTFYKSKYTKGSIEEIKLKGSKEEYLLRFEKLIKSLTNFLTQFNRNKLLQDKDLLLDWVELAKNIVSVLIRFSTNTEFSLNEIKAQSQFKKAKNYLELFKLKKAKKKEFGFIIQSFILSEIKGAATLYSKRKYIASYSVQIVGSNNKFKLFYQPLDSSINISGGPKDFVTKKHKYLIVFQDLKNVKNKDATENRINLLRLNKERKIPLVAYKDDLVSKSLLLSSSPYQLQFLDKYLYRPRGWENIDIKLNEWSFVVEEAYDIEWDLNSKTPKLIPSPKSNRNKLYLAIPFTLKGNVKEPPLDKIVLKSETKKDHSRDKNRLNYPILGVDVGEYGVAWCLTKFDYNQDFSLRDIDIQGKGFIEDRNIGKIKDYFAEIQQKSRKGAYDEDDTTIAKVRENAIGKLRNAIHSILTGSLEGASPVYEDAISNFETGSGKTIKIYNSVKRADTEFKSEADKAEHSLVWGKKDRNQETKYIGRNVSAYASSYTCVNCLHTLFKVKKEDLSNIKILEKDGRIVTMSSPYGPDKKVRGYLSEKEKYEIGYQFKESEEDLKAFRKIVRDFARPPVNKNSEVLEKYAKEILAGNKIEEFRKKRGNSAIFVCPFCQFKADADIQAAFMMAMRGYLRFSGIVPSKENSKNNPQESEDKSLKNSKKQSETGDTFLTKTAEYLQQLRFEIKEKIKEAVKVDF

SEQ ID NO: 120 - protein, 1258 aa, Unknown
  Description of Unknown CasM.21520 sequence
MNEKKTATQRRNARRRRGERARTKSQELRGYRLHDARIEFSGGLGSMRTVKVELLNPDSSREDPQRGQGLQGKVAKAVFDDYRALYGPMNIEDYLSDPDCPSFLGLWVKAVCLGVIMSRKTATDFGELRGGSKSGQAFDSIPEHLRRQLIKLKWLDWYDKGIRKSSSKASRLKSLTDVFANPKQPDQGVMAAWEQGEKLAESSRDIAALGRREFKDKLFAIPPPTSSVVLDDDVKATKVSRDWQWAVDPQFKLPSTDLDITRALEEVDRQWFERLGNNRGMVQQFFAIGDNGNHLNNGLFGHFFASIRSANLADIVAEMGTAFGFSAEERDIVRQRLETLHEYAQGLPEKPVLASRWAEYRTDMTAKLGSWYSNRTSKGAASITQVWGTINTETGEVKDDGLVRTLENIQSDLPDSCSIKEGILQETLDFIGDRRSSTDRAFTDELELYLATLRSDLNTWCQEQSALWEEKQRQVATPASDEKSKKADNPWAGKGSKTDKWLGALHTRIQSSPLFWGVDKLELWKTLANLKQAIRDEIDKLNEQVEVFGRSAYDEPVGKDADSGEGDRRVDQLSYLSARLGDQAHEEVRQRLDAIALALGVKFSERDDLHRFFVSSRARRRAALLAMPNTITVGKLRELADLTPLWERIKKKPEEPRLLADTVALSKVVNSACASRANPSDQIELTTIHSRLDGYSKNIGHTEFISRATVQSTNGAQNTVALDSLVSPRLFYYNFPNIVESAEPHVSHLEVATRGNLGSFEEFAAKEHRTFDRENPQKDSRNRIDSVNPLAVASSRYQIQFFTWWAGLHRSKETALEVGGSFTIAERQVRLDWSQEKPQAVVSEELRVFVSQPFTIVPDDKKRPATSGTRYIGVDIGEYGLAWSCWEFAPGYWNGSVVNPSKVTCLDYGFLAEPGQRRIVERVKKLRESQATKTFTSPDTYIARLRENVVATYQAQLEALMMAYNAQLVFESEISAFETGGNRVKKIYDAIKRSSVFGRSDAEATDNNQHWGKNGNRSSVKDPDKLRLNEAGQVAARVPWAEPVSAWMTSQTCSACGRVYVRAYRGKNSNEPDSGATGEVRYFDNKQQKILTKTIGADTVWVTDQERKEFERGVYNAMRPNAFMPDGRWTAAGEILEAALKSRGTLDGGRGFAGLHLTSKAQVHEYIEGTGKSHRDAHGNSAIFICPYTDCGHIADADLQASYNIALRGFAYAIVRKKHPELFAGSGSSTDGDEGGGKKPQQKQAFIDEIVRAAGRAS

SEQ ID NO: 121 - protein, 895 aa, Unknown
  Description of Unknown CasM.21522 sequence
NPLGITACLLPGFETPDAYRAGRDVTLLYHVQRMQRLLSLDEVKEAYEFVGMHDSALSNFLNGNSNKGFLALLLRGEFDTLARGMMDMTPLWNEHNHDVLMNRLQALGRNAQKLSFTKPRFGNSFADHRKQISGKATAWFSGYCNKLDIAKEQIPLVLEDAKMFMEMLCAVEIDYEKEEFMHLQLSAFIERMERAHERLDADGVVALKKAQYVLPIIREFANPIVQREEAQQWLSLNIELVPDKRFTFKQAFPYLSDQGGADTEKGEQTKFQSVPRFFAEGAHAEYVAFSQAPMIFSVYMENLRLLWDRLIGMERRTPRNWEEHLSKLLYYLYVDIYVECRTDACKKYVRGILETYAHVSRIPEHVPGIKRFTIKPGYTPQGGLTKDQLVQCVLQIAQRLSNAGFEWKRISALERLDLVEVHKKAFAFLVGITHGSVDVSAYNWLNNKSVVQYLDVLKTTDLGGIRLARFLQSCVCATLRGSATRMSEQMLTARFSVQTATTIPQCELVYAVSEYMRKRRFVTPPSHVSRDVLDRKPASIVAQAIYNPEQMAGSMRFIPHRFGYQIQLPELARHESLNNALVVQKPTSTRRYSFARFDAEKGPVLWVESSHYQQQYFDWFFFAPNNRPADVIPQGVSLVVEQDIALQWDFDALVVHMQPVHEPRMYCPQPFKFVPRVESSVMQNRYMGIAPGTNEVAYAVIEVNGTRVSVINCGTFPLAGFANLSRAEEKKQKRERRVGSHAFSPDKRAIVLDNSANKLANQIHACAIQNRARLVWQWTPQTVQIAKGREVDMVYARARKSCAPKNDSDFDAMKTKWGKIWSAKWEDGKHTLSTEVYYAESLFQSACNAPVPPECATALMVALLGRVRDIATPKKWEDDAWRADTLAELFQQITH

SEQ ID NO: 122 - protein, 1184 aa, Unknown
  Description of Unknown CasM.21516 sequence
MFNQKKGYRLHLERIIYSGGEITRSIKYLLASHSDSQKNKELLNNFSQDLYNDDLKIRGCLNLNDLVNNNQIYNLADFWIDSLRAGVIWQSSASSLIDFIKRLNHQETIGEKIFNNANERIKRFFNSEKFIKEIILSEPKRISSKKQAFYNSLFDILKDEFKKQEKNEKIIIDNKAEQLIKEIVDAFYSNDGVFLMEGEEKQNNFWQEKFNIDKNMIKKEKEDILKDVGDITAFIHPPLIILKGDVSQLIDERKKYFSEKDLEEILGLSDNFNAFSHYFNKFFLLLYQDKQEKIFECYQKIFSFSQEDRKRIKDALDFLLEKSKLLGLPKIVNSWSDYRSVFGGKIKSWFSNYLNREDKAKKQEKKIKEGLEKVNKFLLDFIQKNQVDSDLQQEIKFYYDKLNQFINSYQNQEFFHQQELFLLFSDLLAEYREKLNRFYQKYLSDKEKEEKKVDEFPLFKDLFEKYEGPISFYGKTKLEDNKKIIDLTFKTIKVGLNLIRRLLIDLYNSSDFKNSDNNNQERDLRRIFEFLLNKIPATKTFREKYLSILKDNFDQQTYKEMTLKPSRYTFVENIYSRENRKLIELPSKNFEELLSKIIKDLTDFSLSFKNDDLFVDIYLLSDLVELAKTLISLVINYSNKSQFDSYKNELIDDTYQKAKKYLETFKISFFNSKKEANYFYQTRVLSELKGAVALFSKKYYQAKYNIQILKSNEIFPLFVKFSDLLKKEEINDINKLKLIFKKPYRYLIALKKIKFKKKQQQSSVIHLDKKNKDLVLISPQDEDFLFKLTSSFYQLQFLDRFVYPVKKWLNVDITLSEWSFILEKKYKINWDFNNGKPEFSEIDSRLYLNIPFKIKAINQQKILKPKELFLGIDVGEYGVGYALVNFKDEEIKIIKSGFIRSKNIASIRDKYRLLQDRSKKGVYFSSTNVVQEVRENAIGEIRNQIHDILIKNNADLIYEYNISNFETGSGRITKIYDSIKKSDVYAENEADKSVIQHVWGIKKSIASHLSAYGSSYTCSNCGRSIFSFSENDIFSSKVIKRDGNIITIQTPKGEVFAYSKDKKFNIGYSFSQEKNKEEMKNLFMKIVKAYARPPLLKSEVLLTQKKLDREFLEKFKKERGNSAIFVCPFVDCQSLADSDIQAAFIMALRGYLKKKKGKDINYLEESLNYLQNFKGKINFSNLLH

SEQ ID NO: 123 - protein, 1192 aa, Unknown
  Description of Unknown CasM.21466 sequence
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDRGVAEKIEQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPAGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWGTYWEKRKPEDILGISTQVYWTGGIGESCPAVAVALLGHIRATSTQTEWEKEEVVFGRLKKFFPS

SEQ ID NO: 124 - RNA, 68 nt, Artificial Sequence
  Description of Artificial Sequence Synthetic oligonucleotide
cuucgccucguccucggagcaagcuccugugggcgagccuuugaaaaggcuauuaaauacucguauug

SEQ ID NO: 125 - RNA, 56 nt, Artificial Sequence
  Description of Artificial Sequence Synthetic oligonucleotide
uuuuccccaacugaaagguuggaugccuuucaaaaggcuauuaaauacucguauug

SEQ ID NO: 126 - RNA, 59 nt, Artificial Sequence
  Description of Artificial Sequence Synthetic oligonucleotide
auguuccagguucuuucgggagccuuggccuuuaugaaggcuauuaaauacucguauug

SEQ ID NO: 127 - RNA, 83 nt, Artificial Sequence
  Description of Artificial Sequence Synthetic oligonucleotide
gccaguuugggaaaccugggucuuuauuuuuaaagacacaggaauucccgcgucuuuguaaagacuauuaaauacucguauug

SEQ ID NO: 128 - RNA, 57 nt, Artificial Sequence
  Description of Artificial Sequence Synthetic oligonucleotide
cuuuuccuuccccaaaagggaaguugccuuuuaaaaggcuauuaaauacucguauug

SEQ ID NO: 129 - DNA, 7 bp, Artificial Sequence
  Description of Artificial Sequence Synthetic oligonucleotide
  modified_base (1)..(5): a, c, t, g, unknown or other
nnnnntr

SEQ ID NO: 130 - RNA, 11 nt, Artificial Sequence
  Description of Artificial Sequence Synthetic oligonucleotide
gcgaugaaggc

SEQ ID NO: 131 - DNA, 200 bp, Unknown
  Description of Unknown Target sequence
cgcgcgtttcggtgatgacggtgaaaacctctgacacatgcagctcccggagacggtcacagcttgtctgtaagcggatgcctgcccgcagactaatcaataccaaactctggaccgcgtaaactttccagtcgggaaacctgtcgtgccagctgcattaatgaatcggccaacgcgcggggagaggcggtttgcgtatt

SEQ ID NO: 132 - RNA, 15 nt, Artificial Sequence
  Description of Artificial Sequence Synthetic oligonucleotide
agccuuugauaaggc

SEQ ID NO: 133 - RNA, 73 nt, Artificial Sequence
  Description of Artificial Sequence Synthetic oligonucleotide
gggaggauaaguauggauauuuccacaaucuugaaagaaagauuuguuagccuuuaauccauucuccuuuccc

SEQ ID NO: 134 - RNA, 71 nt, Artificial Sequence
  Description of Artificial Sequence Synthetic oligonucleotide
gggaggauaaguauggauauuuccacaaucuugaaagaaagauuuguuagccuuuaauccauucuccuuuc

SEQ ID NO: 135 - RNA, 68 nt, Artificial Sequence
  Description of Artificial Sequence Synthetic oligonucleotide
ggauaaguauggauauuuccacaaucuugaaagaaagauuuguuagccuuuaauccauucuccuuucc

SEQ ID NO: 136 - RNA, 56 nt, Artificial Sequence
  Description of Artificial Sequence Synthetic oligonucleotide
aguauggauauuuccacaaucuugaaagaaagauuuguuagccuuuaauccauucu

SEQ ID NO: 137 - RNA, 50 nt, Artificial Sequence; identical to SEQ ID NO: 33
  Description of Artificial Sequence Synthetic oligonucleotide

SEQ ID NO: 138 - RNA, 50 nt, Artificial Sequence
  Description of Artificial Sequence Synthetic oligonucleotide
auggauauuuccacaaucuugaaagaaagauuuguuagccuucgauccau

SEQ ID NO: 139 - RNA, 40 nt, Artificial Sequence
  Description of Artificial Sequence Synthetic oligonucleotide
uauuuccacaaucuugaaagaaagauuuguuagccuuuaa

SEQ ID NO: 140 - RNA, 45 nt, Artificial Sequence
  Description of Artificial Sequence Synthetic oligonucleotide
uauuuccacaaucuugaaagaaagauuuguuagccuuuaauccau

SEQ ID NO: 141 - RNA, 50 nt, Artificial Sequence
  Description of Artificial Sequence Synthetic oligonucleotide
auggauaaaaggccaaucuugaaagaaagauuuguuagccuuuaauccau

SEQ ID NO: 142 - RNA, 50 nt, Artificial Sequence
  Description of Artificial Sequence Synthetic oligonucleotide
auggauauuuccacaaucuugaaagaaagauuuguaugccuuuaauccau

SEQ ID NO: 143 - RNA, 50 nt, Artificial Sequence
  Description of Artificial Sequence Synthetic oligonucleotide
aagauuuguuagccuuuaauccaugaaagaauggauauuuccacaaucuu

SEQ ID NO: 144 - RNA, 50 nt, Artificial Sequence
  Description of Artificial Sequence Synthetic oligonucleotide
auggauccaauuacaaucuugaaagaaagauuuguuagccuuuaauccau

SEQ ID NO: 145 - RNA, 105 nt, Artificial Sequence; identical to SEQ ID NO: 32
  Description of Artificial Sequence Synthetic polynucleotide

SEQ ID NO: 146 - RNA, 63 nt, Artificial Sequence
  Description of Artificial Sequence Synthetic oligonucleotide
uauuuccacaaucuugaaagaaagauuuguuagccuuugauaaggccaagacccgcgccgagg

SEQ ID NO: 147 - RNA, 28 nt, Artificial Sequence
  Description of Artificial Sequence Synthetic oligonucleotide
gauaaggccaagacccgcgccgagguga

SEQ ID NO: 148 - DNA, 36 bp, Artificial Sequence
  Description of Artificial Sequence Synthetic oligonucleotide
atgttcctgcttcatccccttctacagtggccttat

SEQ ID NO: 149 - DNA, 36 bp, Artificial Sequence
  Description of Artificial Sequence Synthetic oligonucleotide
atgttcctgcttcatgcctttctacagtggccttat

(19) United States
(12) Patent Application Publication
(10) Pub. No.: US 2023/0332218 A1
(43) Pub. Date: Oct. 19, 2023

(54) CASY PROGRAMMABLE NUCLEASES AND RNA COMPONENT SYSTEMS

(71) Applicant: Mammoth Biosciences, Inc., Brisbane, CA (US)

(72) Inventors: Benjamin RAUCH, San Francisco, CA (US); William Douglass WRIGHT, Fairfield, CA (US); Wiputra HARTONO, San Francisco, CA (US); Lucas Benjamin HARRINGTON, San Francisco, CA (US); Clarissa Oriel RHINES, San Mateo, CA (US); Janice Sha CHEN, San Francisco, CA (US); James Paul BROUGHTON, South San Francisco, CA (US); Aaron DELOUGHERY, San Francisco, CA (US)

(21) Appl. No.: 17/919,786

(22) PCT Filed: Apr. 21, 2021
(86) PCT No.: PCT/US2021/028481

§ 371 Date: Oct. 19, 2022

Related U.S. Application Data
(60) Provisional application No. 63/147,567, filed on Feb. 9, 2021.
(60) Provisional application No. 63/013,332, filed on Apr. 21, 2020.

(51) Int. Cl. C12Q 1/6858; C12N 15/11; C12N 9/22; C12N 15/90

(52) U.S. Cl. CPC C12Q 1/6858; C12N 15/11; C12N 9/22; C12N 15/902; C12Q 2600/156; C12N 2310/20; C12N 2800/80; C12N 2310/531

(57) ABSTRACT

The present disclosure provides compositions and methods of use for Type V CRISPR/Cas proteins. Type V CRISPR/Cas nucleases may be configured to bind nucleic acids in a sequence specific manner. Such a binding event may activate the Type V CRISPR/Cas nuclease for sequence non-specific trans-collateral cleavage of single-stranded nucleic acids. Provided herein are methods leveraging Type V CRISPR/Cas nuclease trans-collateral cleavage for identifying nucleic acid sequences.

### CROSS REFERENCE

The present application is a U.S. National Stage Application under 35 U.S.C. § 371 of International Application No. PCT/US2021/028481, filed Apr. 21, 2021, which claims priority to and benefit from U.S. Provisional Application No. 63/147,567 filed Feb. 9, 2021 and U.S. Provisional Application No. 63/013,332 filed on Apr. 21, 2020, the entire contents of each of which are herein incorporated by reference.

### INCORPORATION OF SEQUENCE LISTING

The Sequence Listing associated with this application is provided electronically in XML file format and is hereby incorporated by reference into the specification. The name of the XML file containing the Sequence Listing is MABI_005_02US SeqList_ST25.txt. The XML file is 203,292 bytes and was created on Oct. 17, 2022.

### BACKGROUND

Cas family programmable nucleases may exhibit nuclease activity upon complex formation with a guide nucleic acid and a target nucleic acid. There exists a need for improved guide nucleic acid component systems to enhance and regulate target-dependent nuclease activity of Cas family programmable nucleases.

### SUMMARY

Described herein, in certain embodiments, is a composition comprising a programmable nuclease or a nucleic acid encoding the programmable nuclease; and an engineered guide RNA comprising a crRNA or a nucleic acid encoding the crRNA, wherein a repeat of the crRNA is no more than 24 bases in length. In some embodiments, a sequence of the repeat comprises 5′-AAGGC-3′. In some embodiments, the engineered guide RNA comprises an intermediary RNA. In some embodiments, the intermediary RNA comprises a repeat hybridization region no more than 7 bases complementary to a sequence of the crRNA. In some embodiments, the intermediary RNA comprises a repeat hybridization region no more than 5 bases complementary to a sequence of the crRNA. In some embodiments, the repeat hybridization region is exposed in a bubble within a stem of a hairpin stem-loop structure of the intermediary RNA. In some embodiments, the crRNA comprises a repeat and a spacer. In some embodiments, the composition further comprises a target nucleic acid. In some embodiments, the spacer is complimentary to a target sequence of the target nucleic acid. In some embodiments, the target nucleic acid is DNA. In some embodiments, the DNA is single stranded DNA. In some embodiments, the DNA is double stranded DNA. In some embodiments, the spacer comprises 15 to 20 bases. In some embodiments, the spacer comprises 17 to 19 bases. In some embodiments, the spacer comprises 17 bases. In some embodiments, the repeat comprises 5 to 20 bases. In some embodiments, the repeat comprises 7-8 bases. In some embodiments, the repeat comprises 5 bases. In some embodiments, the repeat further comprises A, U, or C 5′ of the 5′-AAGGC-3′. In some embodiments, the repeat comprises A or U 5′ of the 5′-AAGGC-3′. In some embodiments, the intermediary RNA comprises an RNA hairpin of from 20 to 56 bases. In some embodiments, the intermediary RNA comprises an RNA hairpin of 21 bases. In some embodiments, the intermediary RNA comprises an RNA hairpin of 25 bases. In some embodiments, the intermediary RNA comprises an RNA hairpin of 56 bases. In some embodiments, the repeat hybridization region is positioned at a 3′ end of the RNA hairpin. In some embodiments, a sequence of the repeat hybridization region comprises 5′ GCCUU 3′. In some embodiments, the intermediary RNA comprises a sequence 5′ of the RNA hairpin that hybridizes to a sequence 3′ of the repeat hybridization region. In some embodiments, the intermediary RNA comprises from 50 to 105 bases. In some embodiments, the intermediary RNA comprises 50 bases. In some embodiments, the intermediary RNA comprises a 5′AU sequence adjacent and 5′ of the 5 bases complementary to the sequence of the crRNA. In some embodiments, the target nucleic acid comprises a protospacer adjacent motif (PAM) of TR, or TTR wherein R is A or G. In some embodiments, the target nucleic acid comprises a PAM of TTA. In some embodiments, the target nucleic acid comprises a PAM of TTG. In some embodiments, the engineered guide RNA is a discrete engineered guide RNA system. In some embodiments, the engineered guide RNA is a composite engineered guide RNA. In some embodiments, the crRNA and the intermediary RNA of the composite engineered guide RNA are linked. In some embodiments, the crRNA is adjacent and 3′ of the intermediary RNA. In some embodiments, the composite engineered guide RNA comprises fewer than 100 bases. In some embodiments, the composite engineered guide RNA comprises 50 to 100 bases. In some embodiments, the composite engineered guide RNA comprises 63 bases. In some embodiments, the crRNA is positioned at a 3′ end of the repeat hybridization region of the intermediary RNA. In some embodiments, the composite engineered guide RNA comprises a tetraloop between the 5′-AAGGC-3′ sequence of the crRNA and the repeat hybridization region of the intermediary RNA. In some embodiments, the tetraloop comprises a U, G, A, or any combination thereof. In some embodiments, the tetraloop is 5′-XGAU-3′, where X is any base. In some embodiments, the tetraloop is 5′-UGAU-3′. In some embodiments, the programmable nuclease is a Cas12 protein. In some embodiments, the Cas12 protein is CasY. In some embodiments, the CasY has at least 60%, at least 70%, at least 80%, at least 85%, at least 90%, at least 92%, at least 95%, at least 97%, at least 99%, or 100% sequence identity with any one of SEQ ID NOs: 1-10 and SEQ ID NOs:118-123. In some embodiments, the composition is at a temperature of up to and including 30° C. In some embodiments, the composition is at a temperature of up to and including 37° C. In some embodiments, the composition is at a pH of from 7 to 9. In some embodiments, the composition is at a pH of from 7.1 to 9. In some embodiments, the composition is at a pH of from 8.5 to 9. In some embodiments, the composition is at a pH of about 8.5. In some embodiments, the composition is at a pH of about 8.8.

Described herein, in certain embodiments, is a method of modifying a target nucleic acid, the method comprising contacting any of the compositions described herein to the target nucleic acid. In some embodiments, the modifying comprises introducing a double stranded break in the target nucleic acid. In some embodiments, the programmable nuclease comprises an enzymatically dead programmable nuclease. In some embodiments, the modifying comprises transcriptional activation. In some embodiments, the enzymatically dead programmable nuclease is fused to a transcriptional activator. In some embodiments, the transcriptional activator comprises VP16, VP64, VP48, VP160, a p65 subdomain, an EDLL activation domain, a TAL activation domain, SET1A, SET1B, MLL1 to 5, ASH1, SYMD2, NSD1, JHDM2a/b, UTX, JMJD3, GCN5, PCAF, CBP, p300, TAF1, TIP60/PLIP, MOZ/MYST3, MORF/MYST4, SRC1, ACTR, P160, CLOCK, Ten-Eleven Translocation (TET) dioxygenase 1 (TET1CD), TET1, DME, DML1, DML2, or ROS1. In some embodiments, the modifying comprises transcriptional repression. In some embodiments, the enzymatically dead programmable nuclease is fused to a transcriptional repressor. In some embodiments, the transcriptional repressor comprises a Krüppel associated box (KRAB or SKD); a KOX1 repression domain; a Mad mSIN3 interaction domain (SID); an ERF repressor domain (ERD), a SRDX repression domain, Pr-SET7/8, SUV4-20H1, RIZ1, JMJD2A/JHDM3A, JMJD2B, JMJD2C/GASC1, JMJD2D, JARID1A/RBP2, JARID1B/PLU-1, JARID1C/SMCX, JARID1D/SMCY, HDAC1, HDAC2, HDAC3, HDAC8, HDAC4, HDAC5, HDAC7, HDAC9, SIRT1, SIRT2, HDAC11, HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2, Lamin A, or Lamin B. In some embodiments, the target nucleic acid is a target DNA. In some embodiments, the target DNA is from an animal. In some embodiments, the target DNA is from a plant. In some embodiments, the target DNA is target chromosomal DNA. In some embodiments, the method further comprises administering the composition to a cell. In some embodiments, the method further comprises inducing production of a biologic by the cell. In some embodiments, the method further comprises administering the composition to a subject in need thereof. In some embodiments, the subject is a human.

Described herein, in certain embodiments, is a method of assaying for a target nucleic acid in a sample from a subject, the method comprising: contacting the sample to: any one of the compositions disclosed herein and a detector nucleic acid; and assaying for a signal produced by cleavage of the detector nucleic acid. In some embodiments, the target nucleic acid is DNA. In some embodiments, the target nucleic acid is RNA. In some embodiments, the method further comprises reverse transcribing the RNA prior to the contacting. In some embodiments, the method further comprises amplifying the target nucleic acid prior to the contacting. In some embodiments, the target nucleic acid is viral DNA or bacterial DNA. In some embodiments, the viral DNA is from papovavirus, human papillomavirus (HPV), hepadnavirus, Hepatitis B Virus (HBV), herpesvirus, varicella zoster virus (VZV), epstein-barr virus (EBV), kaposi's sarcoma-associated herpesvirus, adenovirus, poxvirus, or parvovirus, an influenza virus, a respiratory syncytial virus, or a coronavirus. In some embodiments, the target nucleic acid comprises a single nucleotide polymorphism. In some embodiments, the signal is produced in the presence of the target nucleic acid comprising a first variant at the single nucleotide polymorphism, and wherein the signal is higher in the presence of the target nucleic acid comprising the first variant at the single nucleotide polymorphism than in the presence of the target nucleic acid comprising a second variant at the single nucleotide polymorphism. In some embodiments, the method further comprises distinguishing a first variant and a second variant of the single nucleotide polymorphism. In some embodiments, the method further comprises determining a homozygous or heterozygous genotype of the sample for a first variant and a second variant of the target nucleic acid. In some embodiments, the sample is heterozygous for a first variant and a second variant of the target nucleic acid.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a schematic of a target nucleic acid (“target DNA”) having a PAM sequence of “TR,” wherein R is A or G. Also shown is an engineered guide RNA (egRNA) system comprising a discrete egRNA system.

FIG. 1B shows an engineered guide RNA (egRNA) system comprising a composite egRNA complexed with a target nucleic acid and a CasY protein.

FIG. 2A shows a graph of results from 2-hour DETECTR reactions in which the length of the repeat of the crRNA was varied.

FIG. 2B shows a graph of results from DETECTR reactions with 20 nM of the target nucleic acid, in which the length of the repeat of the crRNA was varied.

FIG. 2C shows a graph of results from DETECTR reactions with 20 nM or 1 nM of the target nucleic acid, in which the length of the repeat of the crRNA was varied.

FIG. 2D shows a graph of results from DETECTR reactions in which the length of the spacer of the crRNA was varied.

FIG. 2E shows a graph of results from 50-min DETECTR reactions in which the length of the spacer of the crRNA was varied.

FIG. 2F shows a graph of results from DETECTR reactions in which various repeats either 8 nucleotides in length (AAGGC+3 nucleotides at the 5′ end) or a “universal” AAGGC repeat was tested.

FIG. 3A shows predicted structures of minimized versions of an intermediary RNA (top) and quantitation of each minimized intermediary RNA in a DETECTR reaction (bottom).

FIG. 3B shows classification of the minimized intermediary RNAs of FIG. 3A as functional or non-functional.

FIG. 3C shows a graph of results from DETECTR reactions with various CasY proteins in combination with various crRNA and various intermediary RNA.

FIG. 4A shows schematics of how composite egRNAs were engineered.

FIG. 4B shows a graph of results from DETECTR reactions in which various composite egRNAs were tested with a CasY protein.

FIG. 5A shows a graph of results from DETECTR reactions in which the order of adding various components to the DETECTR reaction was modulated. In Scheme A, the CasY protein was first added, followed by the crRNA, followed by the intermediary RNA. In Scheme B, the CasY protein was first added, followed by the intermediary RNA, followed by the crRNA. In Scheme C, the CasY protein was first added, followed by both RNA components together (crRNA and intermediary RNA).

FIG. 5B shows a graph of results from DETECTR reactions in which two CasY proteins were tested at several pH values. Triplicate reaction traces (time versus absorbance units) for each condition are shown below the graphed data.

FIG. 5C shows an agarose gel of DETECTR assay products to reveal the extent of cis cleavage in the DETECTR reactions. Various nucleic acid species in the reaction are labeled. Triplicate reaction traces (time versus absorbance units) for each condition are shown below the graphed data.

FIG. 6A shows results from genome editing with various CasY proteins targeting a GFP domain. The graphed results show the fraction of cells that still fluoresced in the GFP channel, as determined by flow cytometry, after the GFP domain was targeted with the CasY proteins tested.

FIG. 6B shows results from a comparison of genome editing efficiency of an LbCas12a protein to a CasY protein and a c2c3 protein (also referred to as “Cas12c”) programmable nuclease by measuring the percentage of cells that still fluorescence in the GFP channel, as determined by flow cytometry, after the GFP domain was targeted with the various programmable nucleases tested.

FIG. 7A illustrates genetic variations in exon 3 of the patatin-like phospholipase domain-containing protein 3 (PNPLA3) gene.

FIG. 7B illustrates detection of PNPLA3 alleles using gRNAs to detect the presence or absence of the at-risk allele (rs738409) while ignoring the non-risk allele (rs738408).

FIG. 8 shows the maximum rates (fluorescence detected per minute) of a DETECTR assay detecting wild type (“WT”), at-risk (rs738409), non-risk (rs738409), or both at-risk and non-risk (rs738409+408) alleles of PNPLA3 using different composite egRNAs.

FIG. 9 shows the time to result (minutes) of a DETECTR assay using different pre-amplification conditions (“pre-amp #1” through “pre-amp #5”).

FIG. 10 illustrates an assay workflow for detecting at-risk alleles of a target gene in about 30 minutes.

FIG. 11A shows limit of detection of a DETECTR assay in the presence of decreasing number of copies of genomic DNA (“HeLa DNA”) per reaction.

FIG. 11B shows the limit of detection of a DETECTR assays to detect a wild type (left) or at-risk (right) allele of PNPLA3 in the presence of decreasing copies of DNA (“concentration”) per reaction.

FIG. 12 shows the results of a DETECTR assay to detect different homozygous or heterozygous combinations of PNPLA3 alleles.

FIG. 13A shows the results of a DETECTR assay to detect different PNPLA3 alleles in validated cell lines.

FIG. 13B shows the genotypes of the cell lines used in the assay shown in FIG. 13A.

FIG. 14 shows the results of a DETECTR assay measuring synthetic control samples for different genetic combinations of PNPLA3 alleles.

FIG. 15 shows the results of a DETECTR assay to detect the presence or absence of an at-risk PNPLA3 allele.

FIG. 16 shows the results of a DETECTR assay to determine PNPLA3 genotype of 22 samples (AZ-01 through AZ-22).

FIG. 17A shows a comparison of DETECTR assays detecting the presence or absence of a PNPLA3 mutation (I148M DETECTR positive or I148M DETECTR negative, respectively) to the at-risk genotype encoding for the wild type sequence (rs738409 absent) or the mutant sequence (rs738409 present).

FIG. 17B shows the raw fluorescence of the DETECTR assay to determine PNPLA3 genotype of 22 samples (AZ-01 through AZ-22), shown in FIG. 16, and 10 additional samples (MB-001 through MB-010).

FIG. 18 shows a summary of results from DETECTR assays to detect the presence or absence of an at-risk PNPLA3 allele in blinded samples.

FIG. 19 shows the results of a DETECTR assay testing nucleotide spacer lengths.

FIG. 20 shows the results of a DETECTR assay to test the temperature sensitivity CasY programmable nucleases.

FIG. 21A illustrates PAM preferences for a CasM.21524 protein. FIG. 21B illustrates PAM preferences for a CasM.21518 protein. FIG. 21C illustrates PAM preferences for a CasM.21516 protein.

### DETAILED DESCRIPTION

Disclosed herein are non-naturally occurring compositions and systems comprising at least one of an engineered Cas protein and an engineered guide nucleic acid, which may simply be referred to herein as a Cas protein and a guide nucleic acid, respectively. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some instances, the guide nucleic acid comprises a non-natural nucleobase sequence. In some instances, the non-natural sequence is a nucleobase sequence that is not found in nature. The non-natural sequence may comprise a portion of a naturally occurring sequence, wherein the portion of the naturally occurring sequence is not present in nature absent the remainder of the naturally occurring sequence. In some instances, the guide nucleic acid comprises two naturally occurring sequences arranged in an order or proximity that is not observed in nature. In some instances, compositions and systems comprise a ribonucleotide complex comprising a CRISPR/Cas effector protein and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally occurring Cas protein, wherein the mutation is not found in nature. The Cas protein may also comprise at least one additional amino acid relative to the naturally occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

In some instances, compositions and systems provided herein comprise a multi-vector system encoding a Cas protein and a guide nucleic acid described herein, wherein the guide nucleic acid and the Cas protein are encoded by the same or different vectors. In some embodiments, the engineered guide and the engineered Cas protein are encoded by different vectors of the system.

The present disclosure provides compositions of new RNA components for use with programmable nucleases for genome editing and detection of target nucleic acids in a sample. RNA components disclosed herein include an engineered guide RNA (egRNA)s comprising a CRISPR RNA (crRNA) and an intermediary RNA. crRNAs described herein have been engineered for structure and sequence. For example, the structures disclosed herein are small crRNA sequences, which support high levels of nuclease activity in the programmable nucleases disclosed herein. crRNAs described herein have also been engineered for sequence. For example, particular bases and positions of said bases within the crRNA have been identified, which support high levels of nuclease activity in the programmable nucleases disclosed herein. Intermediary RNAs described herein have been engineered for structure and sequence. For example, the structures disclosed herein are small intermediary RNA sequences, which support high levels of nuclease activity it the programmable nucleases disclosed herein. Intermediary RNAs described herein have also been engineered for sequence. For example, particular bases and positions of said bases within the intermediary RNA have been identified, which support high levels of nuclease activity in the programmable nucleases disclosed herein.

Engineered guide RNA (egRNA) systems disclosed herein include these engineered RNA components (crRNA and intermediary RNA). The present disclosure additionally provides egRNA systems in which the crRNA and intermediary RNA are separate (discrete egRNA systems) and egRNA systems in which the crRNA and intermediary RNA are linked (composite egRNAs).

### RNA Components

The present disclosure provides compositions of RNA components that can be coupled with a programmable nuclease to support high levels of nuclease activity by a programmable nuclease (e.g., a Cas12 nuclease such as CasY, also referred to as “Cas12d”). These RNA components include crRNA and intermediary RNA and form the engineered guide RNA (egRNA) systems described herein. The RNA components of the present disclosure may comprise nucleotides. The term “nucleotide” may be used interchangeably with “nucleotide residue,” “nucleic acid,” “nucleic acid residue,” “base,” or “nucleotide base.” The crRNAs and intermediary RNAs disclosed herein have been engineered for superior activity when used with CasY proteins and have been designed to be used as separate RNA components (referred to as a “discrete egRNA system”) or as linked RNA components (referred to as a “composite egRNA”). A composite egRNA comprises a crRNA and an intermediary RNA in a single polyribonucleotide. A discrete egRNA system (comprising a crRNA and an intermediary RNA) described herein may activate enzymatic activity in a programmable nuclease (e.g., a CasY protein) upon hybridization to a target nucleic acid. A composite egRNA described herein may activate enzymatic activity in a programmable nuclease (e.g., a CasY protein) upon hybridization to a target nucleic acid. Formation of a complex comprising a programmable nuclease (e.g., a CasY protein), a discrete egRNA system or a composite egRNA, and a target nucleic acid may activate trans cleavage activity by the programmable nuclease of collateral nucleic acids (nucleic acids that are not the target nucleic acid). Formation of a complex comprising a programmable nuclease (e.g., a CasY protein), a discrete egRNA system or a composite egRNA, and a target nucleic acid may activate cis cleavage activity by the programmable nuclease of the target nucleic acid.

a. crRNA

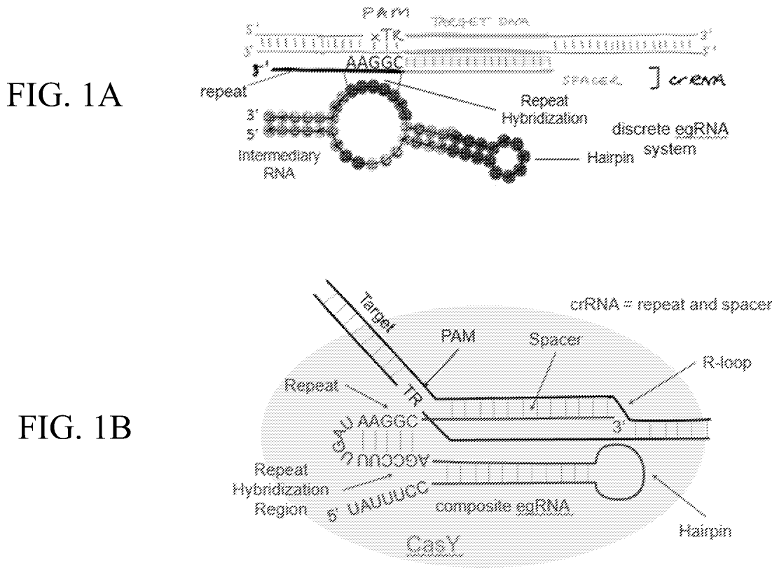

Provided herein are crRNAs that have been engineered to support high levels of programmable nuclease activity. As shown in FIG. 1A and FIG. 1B, a crRNA can comprise a repeat and a spacer. The spacer can have a sequence that hybridizes to a sequence of a target nucleic acid. The sequence of the target nucleic acid that hybridizes to the spacer may also be referred to as the target region. The spacer can have a sequence that is reverse complementary, or sufficiently reverse complementary to allow for hybridization, to a sequence of a target nucleic acid. In some embodiments, a portion of the spacer sequence hybridizes to a sequence of a target nucleic acid. The portion of the spacer sequence can have a sequence that is reverse complementary, or sufficiently reverse complementary to allow for hybridization, to the sequence of the target nucleic acid.

Repeats. A crRNA may comprise a repeat positioned immediately 5′ of the spacer. The repeat may have a length of no more than 25 nucleotides. In some embodiments, the repeat has a length of from 5 to 25 nucleotides. In some embodiments, the repeat has a length of from 5 to 20 nucleotides. In some embodiments, the repeat has a length of from 5 to 15 nucleotides. In some embodiments, the repeat has a length of from 5 to 10 nucleotides. In a preferred embodiment, the repeat has a length of from 5 to 8 nucleotides. The repeat may have a length of no more than 25 nucleotides. In some embodiments, the repeat has a length of no more than 20 nucleotides. In some embodiments, the repeat has a length of no more than 15 nucleotides. In some embodiments, the repeat has a length of no more than 10 nucleotides. In a preferred embodiment, the repeat has a length of no more than 8 nucleotides. In some embodiments, the repeat has a length of about 5, about 6, about 7, about 8, about 9, about 10, about 11, about 12, about 15, about 20, or about 25 nucleotides. In a first preferred embodiment, the repeat has a length of 7 nucleotides. In some embodiments, a repeat sequence with a length of 7 nucleotides may have a sequence of NNAAGGC, wherein N is any nucleotide residue. In a second preferred embodiment, the repeat has a length of 8 nucleotides. In some embodiments, a repeat sequence with a length of 8 nucleotides may have a sequence of NNNAAGGC, wherein N is any nucleotide residue (e.g., A, C, U, or G).

The repeat may comprise a sequence that hybridizes to an intermediary RNA. The sequence that hybridizes to the intermediary RNA may be positioned 5′ of the spacer of the crRNA. The sequence that hybridizes to the intermediary RNA may have a length of about 5 nucleotides. In a preferred embodiment, the sequence that hybridizes to the intermediary RNA may have a sequence of AAGGC. This AAGGC sequence may be a conserved motif across several crRNA repeats disclosed herein. The conserved AAGGC sequence may hybridize with an intermediary RNA. For example, the conserved AAGGC sequence in the repeat may hybridize with a conserved GCCUU sequence in the intermediary RNA. In some embodiments, the repeat and the intermediary RNA are part of a single polyribonucleotide, for example in the composite egRNAs disclosed herein. The repeat may comprise a sequence immediately 5′ of the sequence that hybridizes to the intermediary RNA. The length and nucleotide identity in the sequence immediately 5′ of the sequence that hybridizes to the intermediary RNA can impact programmable nuclease (e.g., a CasY protein) cleavage activity. In some embodiments, the nucleotide sequence of the repeat that may impact programmable nuclease cleavage activity may have a length of from 2 to 20 nucleotides. In some embodiments, the nucleotide sequence of the repeat that may impact programmable nuclease cleavage activity may have a length of from 2 to 15 nucleotides, from 2 to 10 nucleotides, or from 2 to 5 nucleotides. Exemplary sequence of the repeat that may impact programmable nuclease cleavage activity include the sequence AU, the sequence AC, the sequence AG, the sequence AA, the sequence CU, the sequence CC, the sequence CG, the sequence CA, the sequence UU, the sequence UC, the sequence UG, the sequence UA, the sequence GU, the sequence GC, the sequence GG, the sequence GA, the sequence GAU, the sequence AUA, the sequence CCU, the sequence GUG, the sequence UCA, the sequence CCC, or the sequence UUU. In a preferred embodiment, the nucleotide sequence of the repeat that impacts programmable nuclease cleavage activity may have a length of from 2 to 3 nucleotides. In a first preferred embodiment, the nucleotide sequence of the repeat that impacts programmable nuclease cleavage activity is AU. For example, a repeat of the present disclosure may have a sequence of 5′ AUAAGGC 3′. In a second preferred embodiment, the nucleotide sequence of the repeat that impacts programmable nuclease cleavage activity is GAU. For example, a repeat of the present disclosure may have a sequence of 5′ GAUAAGGC 3′.

The repeat may be part of a crRNA. The repeat may be part of the crRNA in a discrete egRNA system. The repeat may be part of the crRNA in a composite egRNA.

Spacers. A crRNA may comprise a spacer positioned immediately 3′ of the repeat. The spacer may hybridize to a sequence of a target nucleic acid. Although 100% reverse complementarity is not needed for hybridization, a spacer can have a sequence that is at least 70% reverse complementary to a region of a target nucleic acid sequence to which the spacer hybridizes. A spacer can have a sequence that is at least 75% reverse complementary, at least 80% reverse complementary, at least 85% reverse complementary, at least 90% reverse complementary, at least 92% reverse complementary, at least 95% reverse complementary, at least 97% reverse complementary, at least 99% reverse complementary, at least 100% reverse complementary, from 70% to 100% reverse complementary, from 80% to 90% reverse complementary, from 85% to 95% reverse complementary, from 75% to 99% reverse complementary, from 90% to 99% reverse complementary, from 90% to 100% reverse complementary, or from 85% to 100% reverse complementary to a region of a target nucleic acid sequence to which the spacer hybridizes.

The spacer can have a length of from 5 to 100 nucleotides. In some embodiments, the spacer has a length of from 5 to 50 nucleotides. In some embodiments, the spacer has a length of from 5 to 25 nucleotides. In some embodiments, the spacer has a length of from 25 to 100 nucleotides. In some embodiments, the spacer has a length of from 50 to 100 nucleotides. In some embodiments, the spacer has a length of from 75 to 100 nucleotides. In a preferred embodiment, the spacer has a length of from 16 to 20 nucleotides. In some embodiments, the spacer has a length of at least 5, at least 10, at least 15, at least 20, at least 25, at least 30, at least 35, at least 40, at least 45, at least 50, at least 55, at least 60, at least 65, at least 70, or at least 75 nucleotides. In a preferred embodiment, the spacer has a length of at least 16 nucleotides. In some embodiments, the spacer has a length of about 16 nucleotides, about 17 nucleotides, about 18 nucleotides, about 19 nucleotides, or about 20 nucleotides. In a first preferred embodiment, the spacer has a length of 17 nucleotides. In a second preferred embodiment, the spacer has a length of 18 nucleotides. In a third preferred embodiment, the spacer has a length of 19 nucleotides.

The spacer may be part of a crRNA. The spacer may be part of the crRNA in a discrete egRNA system. The spacer may be part of the crRNA in a composite egRNA.

Universal Repeat Sequences. The repeat of a crRNA may contain nucleotides that may impart sequence-dependent activation (e.g., sequence-dependent activation of a CasY protein of the present disclosure). In some embodiments, the two or three nucleotides immediately 5′ of the sequence of the repeat that hybridizes to the intermediary RNA may impart sequence-dependent activation of the programmable nuclease. That is, the two or three nucleotides immediately 5′ of the sequence of the region of the repeat that hybridizes to the intermediary RNA may impart sequence-dependent, ortholog-specific activation of programmable nuclease enzymatic activity (cis cleavage activity or trans cleavage activity). For example, a repeat may have a sequence of 5′ AUAAGGC 3′, wherein the two nucleotides at the 5′ end (AU) impart the activity (e.g., trans cleavage activity) of a programmable nuclease (e.g., a CasY protein). As another example, a repeat may have a sequence of 5′ GAUAAGGC 3′, wherein the three nucleotides at the 5′ end (GAU) may impart sequence-dependent activation of the programmable nuclease (e.g., a CasY protein). A repeat lacking these short dinucleotides or trinucleotides at the 5′ end may be a universal repeat sequence. A crRNA comprising a universal repeat may activate two or more programmable nuclease orthologs (e.g., two or more CasY orthologs) when the crRNA is complexed with an intermediary RNA (as a discrete egRNA system or as a composite egRNA), the programmable nuclease, and a target nucleic acid. For example, a crRNA comprising a universal repeat may activate two or more of a CasY3, a CasY10, or a CasY15. An exemplary sequence of a universal repeat may be 5′ AAGGC 3′. For example, a universal repeat sequence may have a sequence of NNNAAGGC, wherein N is any nucleotide residue (e.g., A, C, U, or G). In another example, a universal repeat sequence may have a sequence of NNAAGGC, wherein N is any nucleotide residue. In contrast, a crRNA comprising an ortholog-specific repeat may activate a single programmable nuclease ortholog or a subset of programmable nuclease orthologs. For example, a crRNA comprising an ortholog-specific repeat may activate a CasY3 but not a CasY10 or a CasY15. In another example, a crRNA comprising an ortholog-specific repeat may activate a CasY3 and a CasY10 but not a CasY15. In some embodiments, a crRNA comprising an ortholog-specific repeat may activate a single programmable nuclease ortholog or a subset of programmable nuclease orthologs and inhibit a different programmable nuclease ortholog or a different subset of programmable nuclease orthologs.

A universal repeat may be positioned immediately 5′ of a spacer that hybridizes to a target nucleic acid. In some embodiments, a sequence of a universal repeat may have a length of no more than 5 nucleotides. In some embodiments, a sequence of a universal repeat may have a length of no more than 10 nucleotides. In some embodiments, a sequence of a universal repeat may have a length of no more than 15 nucleotides. In some embodiments, a sequence of a universal repeat may have a length of from 3 to 15 nucleotides. In some embodiments, a sequence of a universal repeat may have a length of from 3 to 10 nucleotides. In some embodiments, a sequence of a universal repeat may have a length of from 3 to 5 nucleotides. In some embodiments, a sequence of a universal repeat may have a length of about 3, about 4, about 5, about 6, about 7, about 8, about 9, or about 10 nucleotides. In a preferred embodiment, a sequence of a universal repeat may have a length of 5 nucleotides.

A crRNA comprising a universal repeat, as disclosed herein, may be used to activate two or more programmable nuclease orthologs having different activities in the presence of a target nucleic acid. A method of modifying or detecting a target nucleic acid with a crRNA comprising a universal repeat may comprise contacting a sample comprising the target nucleic acid with two or more programmable nuclease orthologs and the crRNA comprising a universal repeat and a spacer that hybridizes to a sequence of the target nucleic acid. The crRNA comprising the universal repeat may form a complex with the target nucleic acid and a first programmable nuclease ortholog, thereby activating the first programmable nuclease ortholog. The crRNA comprising the universal repeat may form a complex with the target nucleic acid and a second programmable nuclease ortholog, thereby activating the second programmable nuclease ortholog. In some embodiments, the crRNA comprising the universal repeat may form a complex with the target nucleic acid and a third programmable nuclease ortholog, thereby activating the third programmable nuclease ortholog. The two or more programmable nuclease orthologs may comprise different functions. In some embodiments, the two or more programmable nucleases may comprise fusion proteins. For example, a first programmable nuclease ortholog may comprise a first programmable nuclease (e.g., a CasY protein) fused to a first fusion protein, and a second programmable nuclease ortholog may comprise a second programmable nuclease (e.g., a CasY protein) fused to a second fusion protein. A fusion protein may comprise an activity (e.g., an enzymatic activity) for use in a biochemical assay, such as for research purposes. For example, a fusion protein may be a reporter protein used to visualize the location of a target nucleic acid site. In some embodiments, a programmable nuclease ortholog comprising a reporter protein fusion protein may use used to label or modify multiple target nucleic acids simultaneously. A fusion protein may comprise an activity (e.g., an enzymatic activity) for use in a genome modification strategy. For example, the fusion protein may comprise a base editing activity, transcriptional modulation activity, or any activity to be specifically targeted to a target site. In some embodiments, the first programmable nuclease ortholog may perform a first activity upon activation, and the second programmable nuclease ortholog may perform a second activity upon activation. For example, the first programmable nuclease ortholog may exhibit target cleavage activity upon activation, and the second programmable nuclease may exhibit trans cleavage activity upon activation, thereby enabling simultaneous modification and detection of a target nucleic acid using two programmable nuclease orthologs and a crRNA comprising a universal repeat.

In some embodiments, a programmable nuclease ortholog may be an enzymatically dead programmable nuclease (e.g., a programmable nuclease lacking cis cleavage activity and/or trans cleavage activity). An enzymatically dead programmable nuclease may be capable of binding to a target nucleic acid sequence when complexed with an egRNA (e.g., a discrete egRNA system or a composite egRNA) but that does not catalyze a cis cleavage reaction or a trans cleavage reaction upon binding to the target nucleic acid sequence. In some embodiments, an enzymatically dead programmable nuclease may comprise a point mutation in an endonuclease domain of the programmable nuclease. In some embodiments the enzymatically dead programmable nuclease may be fused to a fusion protein having additional enzymatic activity. The protein having additional activity may catalyze a reaction upon recruitment to the target nucleic acid by the enzymatically dead programmable nuclease. The enzymatically dead programmable nuclease may be a dead Cas12 protein (e.g., a dead CasY protein).

Ortholog-Specific Repeat Sequences. In some embodiments, an ortholog-specific repeat may comprise nucleotides that form sequence-specific interactions with a single programmable nuclease ortholog, a subset of programmable nuclease orthologs, a single intermediary RNA complexed with a programmable nuclease, or a subset of intermediary RNAs complexed with a programmable nuclease. A crRNA comprising the ortholog-specific repeat sequence may activate a programmable nuclease ortholog (e.g., a CasY ortholog) when complexed with the programmable nuclease, an intermediary RNA, and a target nucleic acid. For example, a crRNA comprising an ortholog-specific repeat sequence may activate a CasY3, a CasY10, or a CasY15. The ortholog-specific repeat sequence may comprise about 1, about 2, about 3, about 4, or about 5 nucleotides that form sequence-specific interactions with a programmable nuclease ortholog. In a first preferred embodiment, the ortholog-specific repeat sequence comprises 2 nucleotides that form sequence-specific interactions with a programmable nuclease ortholog. In a second preferred embodiment, the ortholog-specific repeat sequence comprises 3 nucleotides that form sequence-specific interactions with a programmable nuclease ortholog. In some embodiments, an ortholog-specific sequence comprises the nucleotides AU, the nucleotides AC, the nucleotides AG, the nucleotides AA, the nucleotides CU, the nucleotides CC, the nucleotides CG, the nucleotides CA, the nucleotides UU, the nucleotides UC, the nucleotides UG, the nucleotides UA, the nucleotides GU, the nucleotides GC, the nucleotides GG, the nucleotides GA, the nucleotides GAU, the nucleotides AUA, the nucleotides CCU, the nucleotides GUG, the nucleotides UCA, the nucleotides CCC, or the nucleotides UUU immediately 5′ of the sequence that hybridizes to the intermediary RNA. In a first preferred embodiment, an ortholog-specific sequence comprises the nucleotides GAU immediately 5′ of the sequence that hybridizes to the intermediary RNA. In a second preferred embodiment, an ortholog-specific sequence comprises the nucleotides AU immediately 5′ of the sequence that hybridizes to the intermediary RNA.

An ortholog-specific repeat may be positioned immediately 5′ of a spacer that hybridizes to a target nucleic acid. In some embodiments, an ortholog-specific repeat may have a length of no more than 5 nucleotides. In some embodiments, an ortholog-specific repeat may have a length of no more than 10 nucleotides. In some embodiments, an ortholog-specific repeat may have a length of no more than 15 nucleotides. In some embodiments, an ortholog-specific repeat may have a length of from 3 to 15 nucleotides. In some embodiments, an ortholog-specific repeat may have a length of from 3 to 10 nucleotides. In some embodiments, an ortholog-specific repeat may have a length of from 3 to 5 nucleotides. In some embodiments, an ortholog-specific repeat may have a length of about 3, about 4, about 5, about 6, about 7, about 8, about 9, or about 10 nucleotides. In a first preferred embodiment, an ortholog-specific repeat may have a length of 7 nucleotides. In a second preferred embodiment, an ortholog-specific repeat may have a length of 8 nucleotides.

A crRNA comprising an ortholog-specific repeat, as disclosed herein, may be used to activate a single programmable nuclease ortholog in a plurality of programmable nuclease orthologs. A method of modifying or detecting a target nucleic acid with a crRNA comprising an ortholog-specific repeat may comprise contacting a sample comprising the target nucleic acid with two or more programmable nuclease orthologs and the crRNA comprising an ortholog-specific repeat and a spacer that hybridizes to a sequence of the target nucleic acid. The crRNA comprising the ortholog-specific repeat may form a complex with the target nucleic acid and a first programmable nuclease ortholog, thereby activating the first programmable nuclease ortholog. The crRNA comprising the ortholog-specific repeat may not form a complex with a second programmable nuclease ortholog and may not activate the second programmable nuclease ortholog. In some embodiments, a method of modifying or detecting a target nucleic acid with a crRNA comprising an ortholog-specific repeat may comprise contacting a sample comprising the target nucleic acid with two or more programmable nuclease orthologs, a first crRNA comprising a first ortholog-specific repeat and a spacer that hybridizes to a first region of the target nucleic acid, and a second crRNA comprising a second ortholog-specific repeat and a spacer that hybridizes to a second region of the target nucleic acid. The first crRNA may activate a first programmable nuclease having a first activity, and the second crRNA may activate a second programmable nuclease having a second activity. For example, first programmable nuclease may have target cleavage activity and may modify the first target nucleic acid upon activation, and the second programmable nuclease may have trans cleavage activity and may detect the second target nucleic acid upon activation.

crRNAs comprising universal repeats may be used to temporally separate the activity of two or more programmable nuclease orthologs using two different crRNAs. In some embodiments, a programmable nuclease of the two or more programmable nuclease orthologs may be a modified programmable nuclease, as disclosed herein. In some embodiments, temporally separation of programmable nuclease activity may be implemented in vitro or in vivo. A crRNA comprising a universal repeat may direct two or programmable nuclease orthologs to the same region of a target nucleic acid. In some embodiments, the two or more programmable nuclease orthologs may be differentially expressed within a target cell. For example, a first gene encoding a first programmable nuclease ortholog may be under a first inducible promoter, and a second gene encoding a second programmable nuclease ortholog may be under a second inducible promoter. The first programmable nuclease ortholog and the second programmable nuclease ortholog may have different activities. For example, the first programmable nuclease ortholog may exhibit trans cleavage activity upon activation, and the second programmable nuclease ortholog may exhibit target cleavage activity upon activation. In some embodiments, the first programmable nuclease ortholog may be a first CasY protein ortholog (e.g., a CasY3, a CasY10, or a CasY15). In some embodiments, the second programmable nuclease ortholog may be a second CasY ortholog (e.g., a CasY3, a CasY10, or a CasY15).

In some embodiments, a programmable nuclease ortholog may be an enzymatically dead programmable nuclease (e.g., a programmable nuclease lacking endonuclease activity). An enzymatically dead programmable nuclease may be capable of binding to a target nucleic acid sequence when complexed with an egRNA (e.g., a discrete egRNA system or a composite egRNA) but that does not catalyze a cis cleavage reaction or a trans cleavage reaction upon binding to the target nucleic acid sequence. In some embodiments, an enzymatically dead programmable nuclease may comprise a point mutation in an endonuclease domain of the programmable nuclease. In some embodiments the enzymatically dead programmable nuclease may be fused to a fusion protein having additional enzymatic activity. The protein having additional activity may catalyze a reaction upon recruitment to the target nucleic acid by the enzymatically dead programmable nuclease. The enzymatically dead programmable nuclease may be a dead Cas12 protein (e.g., a dead CasY protein).

In some embodiments, crRNAs comprising ortholog-specific repeat may be used to spatially separate the activity of two or more programmable nuclease orthologs along a genome. A first crRNA comprising a first ortholog-specific repeat may direct a first programmable nuclease to a first region of a target nucleic acid, and a second crRNA comprising a second ortholog-specific repeat may direct a second programmable nuclease to a second region of the target nucleic acid, thereby spatially separating the activity of two or more programmable nuclease orthologs. The first region of the target nucleic acid may be spatially separated from the second region of the target nucleic acid by a genomic distance (e.g., a number of bases or a number of centimorgans) along a genome. In some embodiments, a programmable nuclease of the two or more programmable nuclease orthologs may be a modified programmable nuclease, as disclosed herein. The first region and the second region may be positioned a desired distance apart (e.g., a desired number of base pairs apart). In some embodiments, crRNA s comprising ortholog-specific repeat may be used to temporally separate the activity of two or more programmable nuclease orthologs. A first crRNA comprising a first ortholog-specific repeat may be expressed at a first time and direct a first programmable nuclease to a target nucleic acid, and a second crRNA comprising a second ortholog-specific repeat may be expressed and a second time and direct a second programmable nuclease to the target nucleic acid, thereby temporally separating the activity of two or more programmable nuclease orthologs. For example, expression of the first programmable nuclease, the second programmable nuclease, the first crRNA, the second crRNA, the intermediary RNA, or any combination thereof may be controlled using inducible RNA polymerase system, possibly in combination with constitutive or transfection-mediated cellular expression the programmable nuclease or RNA components. Using an inducible RNA polymerase system may enable differential timing for site-specific activation of programmable nuclease activities. The first programmable nuclease ortholog and the second programmable nuclease ortholog may have different activities. For example, the first programmable nuclease may exhibit trans cleavage activity of collateral nucleic acids upon activation, and the second programmable nuclease may exhibit cis cleavage activity of the target nucleic acid upon activation. In some embodiments, the first programmable nuclease ortholog may be a first CasY ortholog (e.g., a CasY3, a CasY10, or a CasY15). In some embodiments, the second programmable nuclease ortholog may be a second CasY ortholog (e.g., a CasY3, a CasY10, or a CasY15). In some embodiments, this approach of combinatorial RNA delivery of multiple CasY proteins may enable spatial or temporal control of programmable nuclease activity, for example, in gene targeting applications where multiple activities, including or in addition to the CasY cis cleavage or trans cleavage activities, are desired at specific settings.

b. Intermediary RNA

Provided herein are intermediary RNAs that have been engineered to have shortened nucleic acid sequences and support high levels of programmable nuclease activity. The intermediary RNA may be separate from, but form a complex with, a crRNA to form a discrete egRNA system. The intermediary RNA may be linked to a crRNA to form a composite egRNA. A programmable nuclease of the present disclosure (e.g., a CasY protein) may be activated to exhibit cleavage activity (e.g., cis cleavage of a target nucleic acid or trans cleavage of a collateral nucleic acid) upon binding of a ribonucleoprotein (RNP) (a complex of a programmable nuclease and egRNA system comprising the intermediary RNA and a crRNA) to a target nucleic acid, in which the spacer of the crRNA hybridizes to the target nucleic acid. In some embodiments, the crRNA and the intermediary RNA are covalently linked in a single polynucleotide (e.g., a composite egRNA). In some embodiments, the crRNA and the intermediary RNA are separate polynucleotides (e.g., a discrete egRNA system). As shown in FIG. 1A and FIG. 1B, an intermediary RNA may comprise a repeat hybridization region and a hairpin region. The repeat hybridization region hybridizes to all or part of the sequence of the repeat of a crRNA. The repeat hybridization region may be positioned 3′ of the hairpin region. The hairpin region may comprise a first sequence, a second sequence that is reverse complementary to the first sequence, and a stem-loop linking the first sequence and the second sequence.

The intermediary RNA may have a length of no more than 105 nucleotides. In some embodiments, the intermediary RNA has a length of from 30 to 120 nucleotides. In some embodiments, the intermediary RNA has a length of from 50 to 105 nucleotides, from 50 to 95 nucleotides, from 50 to 73 nucleotides, from 50 to 71 nucleotides, from 50 to 68 nucleotides, or from 50 to 56 nucleotides. In some embodiments, the intermediary RNA has a length of from 56 to 105 nucleotides, from 56 to 105 nucleotides, from 68 to 105 nucleotides, from 71 to 105 nucleotides, from 73 to 105 nucleotides, or from 95 to 105 nucleotides. In a preferred embodiment, the intermediary RNA has a length of from 40 to 60 nucleotides. In some embodiments, the intermediary RNA has a length of no more than 95 nucleotides. In some embodiments, the intermediary RNA has a length of no more than 73 nucleotides. In some embodiments, the intermediary RNA has a length of no more than 71 nucleotides. In some embodiments, the intermediary RNA has a length of no more than 68 nucleotides. In some embodiments, the intermediary RNA has a length of no more than 56 nucleotides. In a preferred embodiment, the intermediary RNA has a length of no more than 50 nucleotides. In some embodiments, the intermediary RNA has a length of about 50, about 56, about 68, about 71, about 73, about 95, or about 105 nucleotides. In a preferred embodiment, the intermediary RNA has a length of 50 nucleotides.

An exemplary intermediary RNA may comprise, from 5′ to 3′, a 5′ region, a hairpin region, a repeat hybridization region, and a 3′ region. In some embodiments, the 5′ region may hybridize to the 3′ region. In some embodiments, the 5′ region does not hybridize to the 3′ region. In some embodiments, the 3′ region is covalently linked to the crRNA (e.g., through a phosphodiester bond). The 3′ region covalently linked to the crRNA may form a stem-loop structure. In a preferred embodiment, the 3′ region covalently linked to the crRNA may have a sequence of 5′ UGAU 3′.

In some embodiments, an intermediary RNA may comprise an un-hybridized region at the 3′ end of the intermediary RNA. The un-hybridized region may have a length of about 1, about 2, about 3, about 4, about 5, about 6, about 7, about 8, about 9, about 10, about 12, about 14, about 16, about 18, or about 20 nucleotides. In some embodiments, the un-hybridized region may have a length of from 0 to 20 nucleotides.

Repeat Hybridization Region. An intermediary RNA of the present disclosure may comprise a “repeat hybridization region”. This repeat hybridization region may be a sequence that hybridizes to a repeat of a crRNA. Although 100% reverse complementarity is not needed for hybridization, a region that hybridizes to a spacer can have a sequence that is at least 70% reverse complementary to the spacer to which it hybridizes. A region that hybridizes to a spacer can have a sequence that is at least 75% reverse complementary, at least 80% reverse complementary, at least 85% reverse complementary, at least 90% reverse complementary, at least 92% reverse complementary, at least 95% reverse complementary, at least 97% reverse complementary, at least 99% reverse complementary, at least 100% reverse complementary, from 70% to 100% reverse complementary, from 80% to 90% reverse complementary, from 85% to 95% reverse complementary, from 75% to 99% reverse complementary, from 90% to 99% reverse complementary, from 90% to 100% reverse complementary, from 85% to 100% reverse complementary to the spacer to which it hybridizes.

The repeat hybridization region can have a length of about 3, about 4, about 5, about 6, about 7, or about 8 nucleotides. In some embodiments, the repeat hybridization region has a length of 5 nucleotides. In a preferred embodiment, the repeat hybridization region has a sequence of 5′ GCCUU 3′. The GCCUU sequence may be substantially centrally located within the intermediary RNA.

In some embodiments, the intermediary RNA comprises un-hybridized nucleotide sequence (depicted in FIG. 1B as the 5′ UAUUUCC sequence) immediately 5′ of the repeat hybridization region. The un base-paired nucleotides immediately 5′ of the repeat hybridization region may not hybridize to the crRNA and may not hybridize to a region of the intermediary RNA. In some embodiments, the un-hybridized nucleotides immediately 5′ of the repeat hybridization region have a length of about 1, about 2, about 3, about 4, or about 5 nucleotides. In a preferred embodiment, the un-hybridized nucleotides immediately 5′ of the repeat hybridization region have a length of 2 nucleotides. In a preferred embodiment, the un-hybridized nucleotides immediately 5′ of the repeat hybridization region have a sequence of UA.

In some embodiments, the intermediary RNA comprises un-hybridized nucleotides immediately 3′ of the repeat hybridization region. The un base-paired nucleotides immediately 3′ of the repeat hybridization region may not hybridize to the crRNA and may not hybridize to a region of the intermediary RNA. In some embodiments, the un-hybridized nucleotides immediately 3′ of the repeat hybridization region have a length of about 1, about 2, about 3, about 4, or about 5 nucleotides. In a preferred embodiment, the un-hybridized nucleotides immediately 3′ of the repeat hybridization region have a length of 2 nucleotides. In a preferred embodiment, the un-hybridized nucleotides immediately 5′ of the repeat hybridization region have a sequence of UA. In another preferred embodiment, the un-hybridized nucleotides immediately 5′ of the repeat hybridization region have a sequence of CG.

Hairpin Region. An intermediary RNA of the present disclosure may comprise a hairpin region. In a preferred embodiment, the hairpin region may be positioned 5′ of the repeat hybridization region that hybridizes to a repeat of a crRNA. In some embodiments, the hairpin region may be positioned 3′ of the repeat hybridization region. The hairpin region may comprise a first sequence, a second sequence that hybridizes to the first sequence, and stem-loop separating the first sequence and the second sequence. Although 100% reverse complementarity is not needed for hybridization, the first sequence can have a sequence that is at least 70% reverse complementary to the second sequence to which it hybridizes. The first sequence can have a sequence that is at least 75% reverse complementary, at least 80% reverse complementary, at least 85% reverse complementary, at least 90% reverse complementary, at least 92% reverse complementary, at least 95% reverse complementary, at least 97% reverse complementary, at least 99% reverse complementary, 100% reverse complementary, from 70% to 100% reverse complementary, from 80% to 90% reverse complementary, from 85% to 95% reverse complementary, from 75% to 99% reverse complementary, from 90% to 99% reverse complementary, from 90% to 100% reverse complementary, from 85% to 100% reverse complementary to the second sequence to which it hybridizes. In a preferred embodiment, the first sequence comprises a single un-hybridized nucleotide as compared to the second sequence. In some embodiments, the stem loop comprises the region that hybridizes to a repeat of a crRNA.

In some embodiments, the hairpin region may have a length of no more than 60 nucleotides. In some embodiments, the hairpin region may have a length of no more than 56 nucleotides. In a preferred embodiment, the hairpin region may have a length of no more than 21 nucleotides. The hairpin region may have a length of from 15 to 60 nucleotides. In a preferred embodiment, the hairpin region has a length of from 20 to 56 nucleotides. The hairpin region may have a length of about 20 nucleotides, about 21 nucleotides, about 25 nucleotides, about 30 nucleotides, about 35 nucleotides, about 40 nucleotides, about 45 nucleotides, about 50 nucleotides, about 55 nucleotides, or about 56 nucleotides. In a preferred embodiment, the hairpin region has a length of about 21 nucleotides.

An intermediary RNA of the present disclosure may comprise a sequence 5′ of the hairpin region. In some embodiments, a region of the sequence 5′ of the hairpin region hybridizes with a region of the intermediary RNA 3′ of the repeat hybridization region. In some embodiments, the region 5′ of the hairpin region does not hybridize with a region of the intermediary RNA. The region 5′ of the hairpin region may have a length of no more than 25 nucleotides. In some embodiments, the region 5′ of the hairpin region has a length of from 5 to 25 nucleotides. In some embodiments, the region 5′ of the hairpin region has a length of from 6 to 24 nucleotide. In some embodiments, the region 5′ of the hairpin region has a length of from 7 to 20 nucleotide. In a first preferred embodiment, the region 5′ of the hairpin region has a length of from 12 to 25 nucleotides. In a second preferred embodiment, the region 5′ of the hairpin region has a length of no more than 7 nucleotides. In some embodiments, the region 5′ of the hairpin region may have a length of about 5 nucleotides, about 6 nucleotides, about 7 nucleotides, about 12 nucleotides, about 15 nucleotides, about 20 nucleotides, about 24 nucleotides, or about 25 nucleotides. In a first preferred embodiment, the region 5′ of the hairpin region has a length of 12 nucleotides. In a second preferred embodiment, the region 5′ of the hairpin region has a length of 7 nucleotides.

c. Discrete Engineered Guide RNA (egRNA) Systems

The compositions disclosed herein may comprise discrete egRNA systems. A discrete egRNA system, as described herein, may comprise a crRNA and an intermediary RNA. In a discrete egRNA system, the crRNA and the intermediary RNA may be distinct polyribonucleotides. In a discrete egRNA system, the crRNA and the intermediary RNA may not be covalently linked. For example, a first polyribonucleotide comprises the crRNA and a second polynucleotide that is not covalently linked to the first polyribonucleotide comprises the intermediary RNA.

A crRNA in a discrete egRNA system may comprise, from 5′ to 3′, a repeat, a spacer, and a 3′ region. In some embodiments, a crRNA in a discrete egRNA system comprises a repeat and a spacer. The repeat may hybridize to a region of an intermediary RNA. The spacer may hybridize to a region of a target nucleic acid. A crRNA in a discrete egRNA system may have a length of no more than 125 nucleic acids. In some embodiments, the crRNA has a length of from 20 to 100 nucleotides. In some embodiments, the crRNA has a length of from 24 to 75 nucleotides. In some embodiments, the crRNA has a length of from 24 to 50 nucleotides. In some embodiments, the crRNA has a length of from 24 to 40 nucleotides. In some embodiments, the crRNA has a length of from 24 to 30 nucleotides. In a preferred embodiment, the crRNA has a length of from 25 nucleotides to 28 nucleotides. The crRNA may have a length of about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 35 nucleotides, about 40 nucleotides, about 45 nucleotides, or about 50 nucleotides. In some embodiments, a crRNA may comprise a repeat region and a spacer region. A crRNA consisting of a repeat region and a spacer region may be sufficient to promote nuclease activity of a programmable nuclease when complexed with the programmable nuclease and a target nucleic acid.

An intermediary RNA in a discrete egRNA system may comprise, from 5′ to 3′, a 5′ region, a hairpin region, a region that hybridizes to a crRNA, and a 3′ region. In a preferred embodiment, the 5′ end of the 5′ region hybridizes to the 3′ region and the 3′ end of the 3′ region does not hybridize to the 3′ region and does not hybridizes to the region that hybridizes to the crRNA. In some embodiments, an intermediary RNA in a discrete egRNA system may have a length of no more than 105 nucleotides. In some embodiments, the intermediary RNA in a discrete egRNA system has a length of from 30 to 120 nucleotides. In some embodiments, the intermediary RNA in a discrete egRNA system has a length of from 50 to 105 nucleotides, from 50 to 95 nucleotides, from 50 to 73 nucleotides, from 50 to 71 nucleotides, from 50 to 68 nucleotides, or from 50 to 56 nucleotides. In some embodiments, the intermediary RNA in a discrete egRNA system has a length of from 56 to 105 nucleotides, from 56 to 105 nucleotides, from 68 to 105 nucleotides, from 71 to 105 nucleotides, from 73 to 105 nucleotides, or from 95 to 105 nucleotides. In a preferred embodiment, the intermediary RNA in a discrete egRNA system has a length of from 40 to 60 nucleotides. In some embodiments, the intermediary RNA in a discrete egRNA system has a length of no more than 95 nucleotides. In some embodiments, the intermediary RNA in a discrete egRNA system has a length of no more than 73 nucleotides. In some embodiments, the intermediary RNA in a discrete egRNA system has a length of no more than 71 nucleotides. In some embodiments, the intermediary RNA in a discrete egRNA system has a length of no more than 68 nucleotides. In some embodiments, the intermediary RNA in a discrete egRNA system has a length of no more than 56 nucleotides. In a preferred embodiment, the intermediary RNA in a discrete egRNA system has a length of no more than 50 nucleotides. In some embodiments, the intermediary RNA in a discrete egRNA system has a length of about 50, about 56, about 68, about 71, about 73, about 95, or about 105 nucleotides. In a preferred embodiment, the intermediary RNA in a discrete egRNA system has a length of 50 nucleotides.

A programmable nuclease of the present disclosure (e.g., a CasY protein) may be activated to exhibit cleavage activity (e.g., cis cleavage of a target nucleic acid or trans cleavage of a collateral nucleic acid) upon binding of a ribonucleoprotein (RNP) (a complex of a programmable nuclease and discrete egRNA system) to a target nucleic acid, in which the spacer of the crRNA of the discrete egRNA system hybridizes to the target nucleic acid.

d. Composite Engineered Guide RNAs (egRNAs)

The compositions disclosed herein may comprise composite egRNAs. A composite egRNA, as described herein, may comprise a crRNA and an intermediary RNA covalently linked. A composite egRNA may comprise a single polyribonucleotide comprising the crRNA and the intermediary RNA. A crRNA and an intermediary RNA in a composite egRNA may be covalently linked. For example, the crRNA and the intermediary RNA in a composite egRNA may be covalently linked through phosphodiester bond. The intermediary RNA may be 5′ of the crRNA. The intermediary RNA may be 3′ of the crRNA. In a preferred embodiment, the composite egRNA comprises, from 5′ to 3′, an intermediary RNA and a crRNA. In some embodiments, a composite egRNA comprises, from 5′ to 3′, a 5′ region of the intermediary RNA, a hairpin region of the intermediary RNA, a 3′ region of the intermediary RNA, a stem-loop region, a repeat, a spacer, and a 3′ region of the crRNA. In a preferred embodiment, the 3′ region of the intermediary RNA hybridizes to the repeat. In a preferred embodiment, the 5′ region of the intermediary RNA does not form base pair interactions. In a preferred embodiment, the 3′ region of the crRNA forms a hairpin.

A composite egRNA may have a length of no more than 125 nucleotides. In some embodiments, the composite egRNA has a length of from 55 to 125 nucleotides. In some embodiments, the composite egRNA has a length of from 63 to 100 nucleotides. In some embodiments, the composite egRNA has a length of from 63 to 75 nucleotides. In some embodiments, the composite egRNA has a length of from 55 to 100 nucleotides. In some embodiments, the composite egRNA has a length of from 55 to 75 nucleotides. In a preferred embodiment, the composite egRNA has a length of from 60 nucleotides to 70 nucleotides. The composite egRNA may have a length of about 55 nucleotides, about 57 nucleotides, about 59 nucleotides, about 62 nucleotides, about 63 nucleotides, about 64 nucleotides, about 65 nucleotides, about 66 nucleotides, about 68 nucleotides, about 70 nucleotides, about 75 nucleotides, about 80 nucleotides, about 90 nucleotides, or about 100 nucleotides. In a preferred embodiment, a composite egRNA has a length of 63 nucleotides.

A programmable nuclease of the present disclosure (e.g., a CasY protein) may be activated to exhibit cleavage activity (e.g., cis cleavage of a target nucleic acid or trans cleavage of a collateral nucleic acid) upon binding of a ribonucleoprotein (RNP) (a complex of a programmable nuclease and composite egRNA) to a target nucleic acid, in which the spacer of the crRNA of the composite egRNA hybridizes to the target nucleic acid. In some embodiments, the composite egRNA comprises an intermediary RNA and a crRNA covalently linked through a phosphodiester bond.

e. Ribonucleoprotein (RNP) Complexes

A programmable nuclease of the present disclosure (e.g., a CasY protein) may interact with (binds to) a corresponding crRNA and a corresponding intermediary RNA (e.g., a discrete egRNA system or a composite egRNA) to form a ribonucleoprotein (RNP) complex that is targeted to a particular region of target nucleic acid via base pairing between the spacer of the crRNA and a target sequence within the target nucleic acid molecule. For example, an RNP complex may comprise a programmable nuclease and a discrete egRNA system comprising a crRNA and an intermediary RNA. An RNP complex may comprise a programmable nuclease and a composite egRNA. A crRNA may comprise a nucleotide sequence (a spacer sequence) that is complementary to a region of sequence of a target nucleic acid. Thus, a programmable nuclease (e.g., a CasY protein) may form a complex with a crRNA and an intermediary RNA, and the crRNA may provide sequence specificity to the RNP complex via the spacer sequence. The programmable nuclease of the complex may provide the site-specific activity upon interaction with the corresponding target nucleic acid. In other words, the programmable nuclease may be guided to a target site (e.g., stabilized at a target site) within a target nucleic acid sequence by virtue of its association with the crRNA.

The programmable nuclease may be activated upon binding of the RNP complex comprising the programmable nuclease, the crRNA, and the intermediary RNA to the particular region of the target nucleic acid. In some embodiments, the target nucleic acid may be a chromosomal target (e.g., a eukaryotic chromosome, a bacterial chromosome, or a viral chromosome), a gene, a plasmid, an untranslated region, or an artificial sequence. Biding of the RNP complex to the region of the target nucleic acid may activate cis cleavage activity of the programmable nuclease. Biding of the RNP complex to the region of the target nucleic acid may activate trans cleavage activity of the programmable nuclease.

f. Modified Programmable Nucleases

A programmable nuclease (e.g., a CasY protein) of the present disclosure may be a modified programmable nuclease. In some embodiments, a modified programmable nuclease may comprise one or more amino acid mutations compared to a native programmable nuclease. For example, a modified programmable may comprise one or more amino acid mutations that reduce the nuclease activity of the programmable nuclease. The modified programmable nuclease may be an enzymatically dead programmable nuclease (e.g., a dead CasY protein). An enzymatically dead programmable nuclease may form a complex with a crRNA and an intermediary RNA. The complex comprising the enzymatically dead programmable nuclease, the crRNA, and the intermediary RNA may bind to a target nucleic acid.

A modified programmable nuclease may be a chimeric protein. In some embodiments, a chimeric protein may comprise a programmable nuclease of the present disclosure (e.g., a CasY protein or a dead CasY protein) and a heterologous polypeptide. The programmable nuclease and the heterologous polypeptide may be fused via an amino acid linker. The programmable nuclease may be a programmable nuclease with wild type nuclease activity. The programmable nuclease may be a programmable nuclease with reduced nuclease activity (e.g., a dead CasY protein). The heterologous polypeptide may comprise an activity, for example transcriptional activation activity or transcriptional repression activity. In some embodiments, a chimeric protein includes a heterologous polypeptide that has enzymatic activity that modifies a target nucleic acid. For example, the heterologous polypeptide may have nuclease activity such as FokI nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity.

In some cases, a chimeric protein includes a heterologous polypeptide that has enzymatic activity that modifies a polypeptide (e.g., a histone) associated with a target nucleic acid. For example, the heterologous polypeptide may have methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity.

Examples of proteins (or fragments thereof) that can be used in increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

Examples of proteins (or fragments thereof) that can be used in decrease transcription include but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET1CD), TET1, DME, DML1, DML2, ROS1), DNA repair activity, DNA damage (e.g., oxygenation) activity, deamination activity such as that provided by a deaminase (e.g., a cytosine deaminase enzyme such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), and polymerase activity, ligase activity, helicase activity, photolyase activity, and glycosylase activity.

In some cases, the fusion partner has enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, and the like, SET1A, SET1B, MLL1 to 5, ASH1, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

### 1. Programmable Nucleases

The programmable nucleases provided herein (e.g., a type V CRISPR protein) enable the detection or modification of target nucleic acids (e.g., DNA or RNA). The detection or modification of the target nucleic acid is facilitated by a programmable nuclease.

A programmable nuclease can comprise a programmable nuclease capable of being activated when complexed with a discrete egRNA or a composite egRNA, and a target nucleic acid. The programmable nuclease can become activated after binding of the spacer of the crRNA of the discrete egRNA or a composite egRNA to the target nucleic. The activated programmable nuclease can cleave the target nucleic acid, referred to herein as “cis cleavage activity” or “target cleavage activity.” Cis cleavage activity can be specific cleavage of the target nucleic acid.

The programmable nuclease can become activated after binding of the egRNA systems disclosed herein to the target nucleic, in which the activated programmable nuclease can exhibit sequence-dependent cleavage activity, also referred to herein as “cis cleavage activity” or “target cleavage activity.” Target cleavage activity can be specific cleavage of a target nucleic acid at or near the region of the target nucleic acid that hybridizes to the spacer of the crRNA of the egRNA system. Target cleavage may introduce a double stranded break into the target nucleic acid. In some embodiments, target cleavage may introduce a double stranded break with a 5′ overhang into the target nucleic acid. In some embodiments, the target nucleic acid may be modified at or near the double stranded break. For example, a donor nucleic acid may be inserted into the target nucleic acid at the double stranded break. In another example, the programmable nuclease may introduce two double stranded breaks in the target nucleic acid, and the nucleic acid sequence between the two double stranded breaks may be deleted. In still another example, the programmable nuclease may introduce two double stranded breaks in the target nucleic acid, the nucleic acid sequence between the two double stranded breaks may be replaced by a donor nucleic acid sequence.

The programmable nuclease can become activated after binding of the egRNA systems disclosed herein target nucleic, in which the activated programmable nuclease can exhibit sequence-independent cleavage activity, also referred to herein as “trans cleavage activity” or “collateral cleavage activity.” Trans cleavage activity can be non-specific cleavage of nearby single-stranded nucleic acids by the activated programmable nuclease, such as trans cleavage of nucleic acids in a detector nucleic acid, where the detector nucleic acid also comprises a detection moiety. Once the nucleic acid of the detector nucleic acid is cleaved by the activated programmable nuclease, the detection moiety is released from the nucleic acid of the detector nucleic acid and generates a detectable signal. Often the detection moiety is at least one of a fluorophore, a dye, a polypeptide, or a nucleic acid. Sometimes the detection moiety binds to a capture molecule immobilized on a solid surface. The detectable signal can be visualized on the solid surface to assess the presence, the absence, or level of presence of the target nucleic acid. A detectable signal can be a calorimetric, potentiometric, amperometric, optical (e.g., fluorescent, colorimetric, etc.), or piezo-electric signal. Often, the detectable signal is present prior to cleavage of the nucleic acid of the detector nucleic acid and changes upon cleavage of the nucleic acid of the detector nucleic acid. Sometimes, the signal is absent prior to cleavage of the nucleic acid of the detector nucleic acid and is present upon cleavage of the nucleic acid of the detector nucleic acid. The detectable signal can be immobilized on a solid surface for detection.

The programmable nucleases disclosed herein may elicit detector nucleic acid activity upon cleavage of the nucleic acid of the detector nucleic acid. Detector nucleic acid activity refers to trans cleavage activity of the detector nucleic acid. Detector nucleic acid activity may be a calorimetric, potentiometric, amperometric, optical (e.g., fluorescent, colorimetric, etc.), or piezo-electric signal. For example, cleavage of the nucleic acid of the detector nucleic acid by the programmable nuclease may elicit a fluorescent signal. Detector nucleic acid activity may increase or decrease over time in response to a programmable nuclease trans cleavage activity. Detector nucleic acid activity may accumulate over time in response to a programmable nuclease trans cleavage activity. A maximal detector nucleic acid activity may occur when a detector nucleic acid signal (e.g., a calorimetric, potentiometric, amperometric, optical (e.g., fluorescent, colorimetric, etc.), or piezo-electric signal) is highest within a designated assay. In some embodiments, a maximal detector nucleic acid signal may occur when a detector nucleic acid signal reaches a maximum signal, after which the detector nucleic acid signal decreases. In some embodiments, a maximal detector nucleic acid signal may occur when a detector nucleic acid signal increases to saturation after which the signal is no longer increasing.

In some embodiments, the Type V CRISPR/Cas protein is a Cas12 protein. Type V CRISPR/Cas proteins (e.g., Cas12) lack an HNH domain. A Cas12 nuclease of the present disclosure cleaves a nucleic acid via a single catalytic RuvC domain. This single catalytic RuvC domain includes 3 partial RuvC domains (RuvC-I, RuvC-II, and RuvC-III, also referred to herein as subdomains) that are not contiguous with respect to the primary amino acid sequence of the Cas12 protein, but form an RuvC domain once the protein is produced and folds. In some embodiments, a programmable nuclease comprises three partial RuvC domains. In some embodiments, a programmable nuclease comprises an RuvC-I subdomain, an RuvC-II subdomain, and an RuvC-III subdomain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. (Murugan et al., Mol Cell. 2017 Oct. 5; 68(1): 15-25). In some embodiments, the Cas12 protein is a CasY protein. A CasY protein may include an N-terminal domain roughly 800-1000 amino acids in length (e.g., about 815 for CasY1 and about 980 for CasYS), and a C-terminal domain that includes 3 partial RuvC domains (RuvC-I, RuvC-II, and RuvC-III, also referred to herein as subdomains) that are not contiguous with respect to the primary amino acid sequence of the CasY protein, but form a RuvC domain once the protein is produced and folds. Thus, in some cases, a CasY protein (of the subject compositions and/or methods) includes an amino acid sequence with an N-terminal domain (e.g., not including any fused heterologous sequence such as a localization sequence and/or a domain with a catalytic activity) having a length in a range of from 750 to 1050 amino acids (e.g., from 750 to 1025, 750 to 1000, 750 to 950, 775 to 1050, 775 to 1025, 775 to 1000, 775 to 950, 800 to 1050, 800 to 1025, 800 to 1000, or 800 to 950 amino acids). In some cases, a CasY protein (of the subject compositions and/or methods) includes an amino acid sequence having a length (e.g., not including any fused heterologous sequence such as a localization sequence and/or a domain with a catalytic activity) in a range of from 750 to 1050 amino acids (e.g., from 750 to 1025, 750 to 1000, 750 to 950, 775 to 1050, 775 to 1025, 775 to 1000, 775 to 950, 800 to 1050, 800 to 1025, 800 to 1000, or 800 to 950 amino acids) that is N-terminal to a split Ruv C domain (e.g., 3 partial RuvC domains-RuvC-I, RuvC-II, and RuvC-III). In some embodiments, a Cas12 protein may recognize a PAM having a sequence of TR, where R represents any purine (e.g., A or G). In some embodiments, a Cas12 protein may recognize a PAM having a sequence of TN, where N represents any nucleotide (e.g., A, C, T, U, or G). In some embodiments, a Cas12 protein may recognize a PAM having a sequence of TA. In some embodiments, a Cas12 protein may recognize a PAM having a sequence of TG. A Cas12 protein can be a CasY protein (also referred to as a Cas12d protein). A Cas12 protein can be a Cas12 variant (e.g., a CasY variant). In some cases, a suitable Cas12 protein comprises an amino acid sequence having at least 50%, at least 55%, at least 60%, at least 65%, at least 70%, at least 75%, at least 80%, at least 85%, at least 90%, at least 92%, at least 95%, at least 98%, at least 99%, or 100%, amino acid sequence identity to any one of the CasY proteins or variants thereof. Exemplary CasY protein sequences are provided in TABLE 1 (e.g., any one of SEQ ID NOs: 1-10 and SEQ ID NOs: 118-123). In some embodiments, a suitable CasY protein comprises a sequence with at least 70%, at least 75%, at least 80%, at least 85%, at least 90%, at least 92%, at least 95%, at least 97%, at least 98%, at least 99%, or 100%, amino acid sequence identity to any one SEQ ID NOs: 1-10 and SEQ ID NOs: 118-123.

TABLE 1
Exemplary CasY Proteins

| SEQ ID | | |
|---|---|---|
| NO: | Description | Sequence |
| SEQ ID | CasY | MRKKLFKGYILHNKRLVYTGKAAIRSIKYPLVAPNKTAL |
| NO: 1 | | NNLSEKIIYDYEHLFGPLNVASYARNSNRYSLVDFWIDS |
| | | LRAGVIWQSKSTSLIDLISKLEGSKSPSEKIFEQIDFELKN |
| | | KLDKEQFKDIILLNTGIRSSSNVRSLRGRFLKCFKEEFRDT |
| | | EEVIACVDKWSKDLIVEGKSILVSKQFLYWEEEFGIKIFP |
| | | HFKDNHDLPKLTFFVEPSLEFSPHLPLANCLERLKKFDIS |
| | | RESLLGLDNNFSAFSNYFNELFNLLSRGEIKKIVTAVLAV |
| | | SKSWENEPELEKRLHFLSEKAKLLGYPKLTSSWADYRMI |
| | | IGGKIKSWHSNYTEQLIKVREDLKKHQIALDKLQEDLKK |
| | | VVDSSLREQIEAQREALLPLLDTMLKEKDFSDDLELYRFI |
| | | LSDFKSLLNGSYQRYIQTEEERKEDRDVTKKYKDLYSNL |
| | | RNIPRFFGESKKEQFNKFINKSLPTIDVGLKILEDIRNALE |
| | | TVSVRKPPSITEEYVTKQLEKLSRKYKINAFNSNRFKQIT |
| | | EQVLRKYNNGELPKISEVFYRYPRESHVAIRILPVKISNPR |
| | | KDISYLLDKYQISPDWKNSNPGEVVDLIEIYKLTLGWLLS |
| | | CNKDFSMDFSSYDLKLFPEAASLIKNFGSCLSGYYLSKMI |
| | | FNCITSEIKGMITLYTRDKFVVRYVTQMIGSNQKFPLLCL |
| | | VGEKQTKNFSRNWGVLIEEKGDLGEEKNQEKCLIFKDK |
| | | TDFAKAKEVEIFKNNIWRIRTSKYQIQFLNRLFKKTKEW |
| | | DLMNLVLSEPSLVLEEEWGVSWDKDKLLPLLKKEKSCE |
| | | ERLYYSLPLNLVPATDYKEQSAEIEQRNTYLGLDVGEFG |
| | | VAYAVVRIVRDRIELLSWGFLKDPALRKIRERVQDMKK |
| | | KQVMAVFSSSSTAVARVREMAIHSLRNQIHSIALAYKAK |
| | | IIYEISISNFETGGNRMAKIYRSIKVSDVYRESGADTLVSE |
| | | MIWGKKNKQMGNHISSYATSYTCCNCARTPFELVIDND |
| | | KEYEKGGDEFIFNVGDEKKVRGFLQKSLLGKTIKGKEVL |
| | | KSIKEYARPPIREVLLEGEDVEQLLKRRGNSYIYRCPFCG |
| | | YKTDADIQAALNIACRGYISDNAKDAVKEGERKLDYILE |
| | | VRKLWEKNGAVLRSAKFL |
| SEQ ID | CasY | MQKVRKTLSEVHKNPYGTKVRNAKTGYSLQIERLSYTG |
| NO: 2 | | KEGMRSFKIPLENKNKEVFDEFVKKIRNDYISQVGLLNL |
| | | SDWYEHYQEKQEHYSLADFWLDSLRAGVIFAHKETEIK |
| | | NLISKIRGDKSIVDKFNASIKKKHADLYALVDIKALYDFL |
| | | TSDARRGLKTEEEFFNSKRNTLFPKFRKKDNKAVDLWV |
| | | KKFIGLDNKDKLNFTKKFIGFDPNPQIKYDHTFFFHQDIN |
| | | FDLERITTPKELISTYKKFLGKNKDLYGSDETTEDQLKM |
| | | VLGFHNNHGAFSKYFNASLEAFRGRDNSLVEQIINNSPY |
| | | WNSHRKELEKRIIFLQVQSKKIKETELGKPHEYLASFGG |
| | | KFESWVSNYLRQEEEVKRQLFGYEENKKGQKKFIVGNK |
| | | QELDKIIRGTDEYEIKAISKETIGLTQKCLKLLEQLKDSVD |
| | | DYTLSLYRQLIVELRIRLNVEFQETYPELIGKSEKDKEKD |
| | | AKNKRADKRYPQIFKDIKLIPNFLGETKQMVYKKFIRSA |
| | | DILYEGINFIDQIDKQITQNLLPCFKNDKERIEFTEKQFET |
| | | LRRKYYLMNSSRFHHVIEGIINNRKLIEMKKRENSELKTF |
| | | SDSKFVLSKLFLKKGKKYENEVYYTFYINPKARDQRRIK |
| | | IVLDINGNNSVGILQDLVQKLKPKWDDIIKKNDMGELID |
| | | AIEIEKVRLGILIALYCEHKFKIKKELLSLDLFASAYQYLE |
| | | LEDDPEELSGTNLGRFLQSLVCSEIKGAINKISRTEYIERY |
| | | TVQPMNTEKNYPLLINKEGKATWHIAAKDDLSKKKGGG |
| | | TVAMNQKIGKNFFGKQDYKTVFMLQDKRFDLLTSKYH |
| | | LQFLSKTLDTGGGSWWKNKNIDLNLSSYSFIFEQKVKVE |
| | | WDLTNLDHPIKIKPSENSDDRRLFVSIPFVIKPKQTKRKD |
| | | LQTRVNYMGIDIGEYGLAWTIINIDLKNKKINKISKQGFI |
| | | YEPLTHKVRDYVATIKDNQVRGTFGMPDTKLARLRENA |
| | | ITSLRNQVHDIAMRYDAKPVYEFEISNFETGSNKVKVIY |
| | | DSVKRADIGRGQNNTEADNTEVNLVWGKTSKQFGSQIG |
| | | AYATSYICSFCGYSPYYEFENSKSGDEEGARDNLYQMK |
| | | KLSRPSLEDFLQGNPVYKTFRDFDKYKNDQRLQKTGDK |
| | | DGEWKTHRGNTAIYACQKCRHISDADIQASYWIALKQV |
| | | VRDFYKDKEMDGDLIQGDNKDKRKVNELNRLIGVHKD |
| | | VPIINKNLITSLDINLL |
| SEQ ID | CasY3 | MKAKKSFYNQKRKFGKRGYRLHDERIAYSGGIGSMRSI |
| NO: 3 | | KYELKDSYGIAGLRNRIADATISDNKWLYGNINLNDYLE |
| | | WRSSKTDKQIEDGDRESSLLGFWLEALRLGFVFSKQSHA |
| | | PNDFNETALQDLFETLDDDLKHVLDRKKWCDFIKIGTPK |
| | | TNDQGRLKKQIKNLLKGNKREEIEKTLNESDDELKEKIN |
| | | RIADVFAKNKSDKYTIFKLDKPNTEKYPRINDVQVAFFC |
| | | HPDFEEITERDRTKTLDLIINRFNKRYEITENKKDDKTSN |
| | | RMALYSLNQGYIPRVLNDLFLFVKDNEDDFSQFLSDLEN |
| | | FFSFSNEQIKIIKERLKKLKKYAEPIPGKPQLADKWDDYA |
| | | SDFGGKLESWYSNRIEKLKKIPESVSDLRNNLEKIRNVLK |
| | | KQNNASKILELSQKIIEYIRDYGVSFEKPEIIKFSWINKTK |
| | | DGQKKVFYVAKMADREFIEKLDLWMADLRSQLNEYNQ |
| | | DNKVSFKKKGKKIEELGVLDFALNKAKKNKSTKNENG |
| | | WQQKLSESIQSAPLFFGEGNRVRNEEVYNLKDLLFSEIK |
| | | NVENILMSSEAEDLKNIKIEYKEDGAKKGNYVLNVLARF |
| | | YARFNEDGYGGWNKVKTVLENIAREAGTDFSKYGNNN |
| | | NRNAGRFYLNGRERQVFTLIKFEKSITVEKILELVKLPSL |
| | | LDEAYRDLVNENKNHKLRDVIQLSKTIMALVLSHSDKE |
| | | KQIGGNYIHSKLSGYNALISKRDFISRYSVQTTNGTQCKL |
| | | AIGKGKSKKGNEIDRYFYAFQFFKNDDSKINLKVIKNNS |
| | | HKNIDFNDNENKINALQVYSSNYQIQFLDWFFEKHQGK |
| | | KTSLEVGGSFTIAEKSLTIDWSGSNPRVGFKRSDTEEKRV |
| | | FVSQPFTLIPDDEDKERRKERMIKTKNRFIGIDIGEYGLA |
| | | WSLIEVDNGDKNNRGIRQLESGFITDNQQQVLKKNVKS |
| | | WRQNQIRQTFTSPDTKIARLRESLIGSYKNQLESLMVAK |
| | | KANLSFEYEVSGFEVGGKRVAKIYDSIKRGSVRKKDNNS |
| | | QNDQSWGKKGINEWSFETTAAGTSQFCTHCKRWSSLAI |
| | | VDIEEYELKDYNDNLFKVKINDGEVRLLGKKGWRSGEK |
| | | IKGKELFGPVKDAMRPNVDGLGMKIVKRKYLKLDLRD |
| | | WVSRYGNMAIFICPYVDCHHISHADKQAAFNIAVRGYL |
| | | KSVNPDRAIKHGDKGLSRDFLCQEEGKLNFEQIGLLI |
| SEQ ID | CasY | MSKRHPRISGVKGYRLHAQRLEYTGKSGAMRTIKYPLY |
| NO: 4 | | SSPSGGRTVPREIVSAINDDYVGLYGLSNFDDLYNAEKR |
| | | NEEKVYSVLDFWYDCVQYGAVFSYTAPGLLKNVAEVR |
| | | GGSYELTKTLKGSHLYDELQIDKVIKFLNKKEISRANGSL |
| | | DKLKKDIIDCFKAEYRERHKDQCNKLADDIKNAKKDAG |
| | | ASLGERQKKLFRDFFGISEQSENDKPSFTNPLNLTCCLLP |
| | | FDTVNNNRNRGEVLFNKLKEYAQKLDKNEGSLEMWEY |
| | | IGIGNSGTAFSNFLGEGFLGRLRENKITELKKAMMDITDA |
| | | WRGQEQEEELEKRLRILAALTIKLREPKFDNHWGGYRS |
| | | DINGKLSSWLQNYINQTVKIKEDLKGHKKDLKKAKEMI |
| | | NRFGESDTKEEAVVSSLLESIEKIVPDDSADDEKPDIPAIA |
| | | TYRRFLSDGRLTLNRFVQREDVQEALIKERLEAEKKKKP |
| | | KKRKKKSDAEDEKETIDFKELFPHLAKPLKLVPNFYGDS |
| | | KRELYKKYKNAAIYTDALWKAVEKIYKSAFSSSLKNSFF |
| | | DTDFDKDFFIKRLQKIFSVYRRFNTDKWKPIVKNSFAPY |
| | | CDIVSLAENEVLYKPKQSRSRKSAAIDKNRVRLPSTENIA |
| | | KAGIALARELSVAGFDWKDLLKKEEHEEYIDLIELHKTA |
| | | LALLLAVTETQLDISALDFVENGTVKDFMKTRDGNLVL |
| | | EGRFLEMFSQSIVFSELRGLAGLMSRKEFITRSAIQTMNG |
| | | KQAELLYIPHEFQSAKITTPKEMSRAFLDLAPAEFATSLE |
| | | PESLSEKSLLKLKQMRYYPHYFGYELTRTGQGIDGGVAE |
| | | NALRLEKSPVKKREIKCKQYKTLGRGQNKIVLYVRSSYY |
| | | QTQFLEWFLHRPKNVQTDVAVSGSFLIDEKKVKTRWNY |
| | | DALTVALEPVSGSERVFVSQPFTIFPEKSAEEEGQRYLGI |
| | | DIGEYGIAYTALEITGDSAKILDQNFISDPQLKTLREEVK |
| | | GLKLDQRRGTFAMPSTKIARIRESLVHSLRNRIHHLALK |
| | | HKAKIVYELEVSRFEEGKQKIKKVYATLKKADVYSEIDA |
| | | DKNLQTTVWGKLAVASEISASYTSQFCGACKKLWRAE |
| | | MQVDETITTQELIGTVRVIKGGTLIDAIKDFMRPPIFDEN |
| | | DTPFPKYRDFCDKHHISKKMRGNSCLFICPFCRANADAD |
| | | IQASQTIALLRYVKEEKKVEDYFERFRKLKNIKVLGQMK |
| | | KI |
| SEQ ID | CasY | MKRILNSLKVAALRLLFRGKGSELVKTVKYPLVSPVQG |
| NO: 5 | | AVEELAEAIRHDNLHLFGQKEIVDLMEKDEGTQVYSVV |
| | | DFWLDTLRLGMFFSPSANALKITLGKFNSDQVSPFRKVL |
| | | EQSPFFLAGRLKVEPAERILSVEIRKIGKRENRVENYAAD |
| | | VETCFIGQLSSDEKQSIQKLANDIWDSKDHEEQRMLKAD |
| | | FFAIPLIKDPKAVTEEDPENETAGKQKPLELCVCLVPELY |
| | | TRGFGSIADFLVQRLTLLRDKMSTDTAEDCLEYVGIEEE |
| | | KGNGMNSLLGTFLKNLQGDGFEQIFQFMLGSYVGWQG |
| | | KEDVLRERLDLLAEKVKRLPKPKFAGEWSGHRMFLHGQ |
| | | LKSWSSNFFRLFNETRELLESIKSDIQHATMLISYVEEKG |
| | | GYHPQLLSQYRKLMEQLPALRTKVLDPEIEMTHMSEAV |
| | | RSYIMIHKSVAGFLPDLLESLDRDKDREFLLSIFPRIPKID |
| | | KKTKEIVAWELPGEPEEGYLFTANNLFRNFLENPKHVPR |
| | | FMAERIPEDWTRLRSAPVWFDGMVKQWQKVVNQLVES |
| | | PGALYQFNESFLRQRLQAMLTVYKRDLQTEKFLKLLAD |
| | | VCRPLVDFFGLGGNDIIFKSCQDPRKQWQTVIPLSVPAD |
| | | VYTACEGLAIRLRETLGFEWKNLKGHEREDFLRLHQLL |
| | | GNLLFWIRDAKLVVKLEDWMNNPCVQEYVEARKAIDL |
| | | PLEIFGFEVPIFLNGYLFSELRQLELLLRRKSVMTSYSVKT |
| | | TGSPNRLFQLVYLPLNPSDPEKKNSNNFQERLDTPTGLSR |
| | | RFLDLTLDAFAGKLLTDPVTQELKTMAGFYDHLFGFKLP |
| | | CKLAAMSNHPGSSSKMVVLAKPKKGVASNIGFEPIPDPA |
| | | HPVFRVRSSWPELKYLEGLLYLPEDTPLTIELAETSVSCQ |
| | | SVSSVAFDLKNLTTILGRVGEFRVTADQPFKLTPIIPEKEE |
| | | SFIGKTYLGLDAGERSGVGFAIVTVDGDGYEVQRLGVH |
| | | EDTQLMALQQVASKSLKEPVFQPLRKGTFRQQERIRKSL |
| | | RGCYWNFYHALMIKYRAKVVHEESVGSSGLVGQWLRA |
| | | FQKDLKKADVLPKKGGKNGVDKKKRESSAQDTLWGGA |
| | | FSKKEEQQIAFEVQAAGSSQFCLKCGWWFQLGMREVNR |
| | | VQESGVVLDWNRSIVTFLIESSGEKVYGFSPQQLEKGFRP |
| | | DIETFKKMVRDFMRPPMFDRKGRPAAAYERFVLGRRHR |
| | | RYRFDKVFEERFGRSALFICPRVGCGNFDHSSEQSAVVL |
| | | ALIGYIADKEGMSGKKLVYVRLAELMAEWKLKKLERSR |
| | | VEEQSSAQ |
| SEQ ID | CasY | MAESKQMQCRKCGASMKYEVIGLGKKSCRYMCPDCGN |
| NO: 6 | | HTSARKIQNKKKRDKKYGSASKAQSQRIAVAGALYPDK |
| | | KVQTIKTYKYPADLNGEVHDSGVAEKIAQAIQEDEIGLL |
| | | GPSSEYACWIASQKQSEPYSVVDFWFDAVCAGGVFAYS |
| | | GARLLSTVLQLSGEESVLRAALASSPFVDDINLAQAEKF |
| | | LAVSRRTGQDKLGKRIGECFAEGRLEALGIKDRMREFVQ |
| | | AIDVAQTAGQRFAAKLKIFGISQMPEAKQWNNDSGLTV |
| | | CILPDYYVPEENRADQLVVLLRRLREIAYCMGIEDEAGF |
| | | EHLGIDPGALSNFSNGNPKRGFLGRLLNNDIIALANNMS |
| | | AMTPYWEGRKGELIERLAWLKHRAEGLYLKEPHFGNS |
| | | WADHRSRIFSRIAGWLSGCAGKLKIAKDQISGVRTDLFL |
| | | LKRLLDAVPQSAPSPDFIASISALDRFLEAAESSQDPAEQ |
| | | VRALYAFHLNAPAVRSIANKAVQRSDSQEWLIKELDAV |
| | | DHLEFNKAFPFFSDTGKKKKKGANSNGAPSEEEYTETES |
| | | IQQPEDAEQEVNGQEGNGASKNQKKFQRIPRFFGEGSRS |
| | | EYRILTEAPQYFDMFCNNMRAIFMQLESQPRKAPRDFKC |
| | | FLQNRLQKLYKQTFLNARSNKCRALLESVLISWGEFYTY |
| | | GANEKKFRLRHEASERSSDPDYVVQQALEIARRLFLFGF |
| | | EWRDCSAGERVDLVEIHKKAISFLLAITQAEVSVGSYNW |
| | | LGNSTVSRYLSVAGTDTLYGTQLEEFLNATVLSQMRGL |
| | | AIRLSSQELKDGFDVQLESSCQDNLQHLLVYRASRDLAA |
| | | CKRATCPAELDPKILVLPVGAFIASVMKMIERGDEPLAG |
| | | AYLRHRPHSFGWQIRVRGVAEVGMDQGTALAFQKPTES |
| | | EPFKIKPFSAQYGPVLWLNSSSYSQSQYLDGFLSQPKNW |
| | | SMRVLPQAGSVRVEQRVALIWNLQAGKMRLERSGARA |
| | | FFMPVPFSFRPSGSGDEAVLAPNRYLGLFPHSGGIEYAVV |
| | | DVLDSAGFKILERGTIAVNGFSQKRGERQEEAHREKQRR |
| | | GISDIGRKKPVQAEVDAANELHRKYTDVATRLGCRIVV |
| | | QWAPQPKPGTAPTAQTVYARAVRTEAPRSGNQEDHAR |
| | | MKSSWGYTWGTYWEKRKPEDILGISTQVYWTGGIGESC |
| | | PAVAVALLGHIRATSTQTEWEKEEVVFGRLKKFFPS |
| SEQ ID | CasY | MAESKQMQCRKCGASMKYEVIGLGKKSCRYMCPDCGN |
| NO: 7 | | HTSARKIQNKKKRDKKYGSASKAQSQRIAVAGALYPDK |
| | | KVQTIKTYKYPADLNGEVHDRGVAEKIEQAIQEDEIGLL |
| | | GPSSEYACWIASQKQSEPYSVVDFWFDAVCAGGVFAYS |
| | | GARLLSTVLQLSGEESVLRAALASSPFVDDINLAQAEKF |
| | | LAVSRRTGQDKLGKRIGECFAEGRLEALGIKDRMREFVQ |
| | | AIDVAQTAGQRFAAKLKIFGISQMPEAKQWNNDSGLTV |
| | | CILPDYYVPEENRADQLVVLLRRLREIAYCMGIEDEAGF |
| | | EHLGIDPGALSNFSNGNPKRGFLGRLLNNDIIALANNMS |
| | | AMTPYWEGRKGELIERLAWLKHRAEGLYLKEPHFGNS |
| | | WADHRSRIFSRIAGWLSGCAGKLKIAKDQISGVRTDLFL |
| | | LKRLLDAVPQSAPSPDFIASISALDRFLEAAESSQDPAEQ |
| | | VRALYAFHLNAPAVRSIANKAVQRSDSQEWLIKELDAV |
| | | DHLEFNKAFPFFSDTGKKKKKGANSNGAPSEEEYTETES |
| | | IQQPEDAEQEVNGQEGNGASKNQKKFQRIPRFFGEGSRS |
| | | EYRILTEAPQYFDMFCNNMRAIFMQLESQPRKAPRDFKC |
| | | FLQNRLQKLYKQTFLNARSNKCRALLESVLISWGEFYTY |
| | | GANEKKFRLRHEASERSSDPDYVVQQALEIARRLFLFGF |
| | | EWRDCSAGERVDLVEIHKKAISFLLAITQAEVSVGSYNW |
| | | LGNSTVSRYLSVAGTDTLYGTQLEEFLNATVLSQMRGL |
| | | AIRLSSQELKDGFDVQLESSCQDNLQHLLVYRASRDLAA |
| | | CKRATCPAELDPKILVLPAGAFIASVMKMIERGDEPLAG |
| | | AYLRHRPHSFGWQIRVRGVAEVGMDQGTALAFQKPTES |
| | | EPFKIKPFSAQYGPVLWLNSSSYSQSQYLDGFLSQPKNW |
| | | SMRVLPQAGSVRVEQRVALIWNLQAGKMRLERSGARA |
| | | FFMPVPFSFRPSGSGDEAVLAPNRYLGLFPHSGGIEYAVV |
| | | DVLDSAGFKILERGTIAVNGFSQKRGERQEEAHREKQRR |
| | | GISDIGRKKPVQAEVDAANELHRKYTDVATRLGCRIVV |
| | | QWAPQPKPGTAPTAQTVYARAVRTEAPRSGNQEDHAR |
| | | MKSSWGYTWSTYWEKRKPEDILGISTQVYWTGGIGESC |
| | | PAVAVALLGHIRATSTQTEWEKEEVVFGRLKKFFPS |
| SEQ ID | CasY | MKRIAKFRHDKPVKREAWSKGYRVHKNRIINKVTRSIK |
| NO: 8 | Ortholog | YPLVVKDEWKKRLIDDAAHDYRWLVGPINYSDWCRDP |
| | | NQYSILEFWIDFLCVGGVFQSSHSNICRLAIQLSGGSVFE |
| | | QEWKDLSPFVRANLIQGIKPAEFIGFLTAEFRSSSNPKNFI |
| | | SKFFEGSNEDLESLTNEFASIVDFIKAKDISLLRKSLPSCK |
| | | KIAPNLWEKAVGSHSTNELLKLLTKYTRVMLVAEPSHS |
| | | DRVFSQTVLQSNDQDDPELTGPLPSHKVGKASYLFIPEFI |
| | | REVNLDKISKLDLSAKSKLAVEQVKKLSELTSDFKQIEN |
| | | QSEAYFGLSTSFNELSNFLGILIRTLRNAPEAILKDQIALC |
| | | APLDKDILKITLDWLCDRAQALPENPRFETNWAEYRSYL |
| | | GGKIKSWFSNYENFFEIPQAASSQQNNNREKKLGNRSAI |
| | | RALNLKKEAFEKARETFKGDKGTLEKIDLAYRLLGSISPE |
| | | VLQCDEGLKLYQQFNDELLVLNETINQKFQDAKRDIKA |
| | | KKEKESFEKLQRNLSSPLPRIPEFFGERAKKGYQKARVSP |
| | | KLARHLLECLNDWLARFAKVEESAFSEKEFQRILDWLRT |
| | | SDFLPVFIRKSKDPPSWLRYIARVATGKYYFWVSEYSRK |
| | | RVQIIDKPIAQNPLKELISWFLLNKDAFSRDNELFKGLSS |
| | | KMVTLARIMAGILRDRGEGLKELQAMTSKLDNIGLLHPS |
| | | FSVPVTDSLKDAAFYRAFFSELEGLLNIGRSRLIIERITLQS |
| | | QQSKNKKTRRPLMPEPFINEDKEVFLAFPKFETKNKVKG |
| | | TRVVYNSPDEVNWLLSPIRSSKGQLSFMFRCLSEDAKIM |
| | | TTSGGCSYIVEFKKLLEAQEEVLSIHDCDIIPRAFVSIPFTL |
| | | ERESEETKPDWKPNRFMGVDIGEYAVAYCVIEKGTDSIE |
| | | ILDCGIVRNGAHRVLKEKVDRLKRRQRSMTFGAMDTSI |
| | | AAARESLVGNYRNRLHAIALKHGAKLVYEYEVSAFESG |
| | | GNRIKKVYETLKKSDCTGETEADKNARKHIWGETNAVG |
| | | DQIGAGWTSQTCAKCGRSFGADLKAGNFGVAVPVPEKV |
| | | EDSKGHYAYHEFPFEDGLKVRGFLKPNKIISDQKELAKA |
| | | VHAYMRPPLVALGKRKLPKNARYRRGNSSLFRCPFSDC |
| | | GFTADADIQAAYNIAVKQLYKPKKGYPKERKWQDFVIL |
| | | KPKEPSKLFDKQFYRPN |
| SEQ ID | CasY10 | MKDSKINAPININANNVSKNKTPKKKPRRKSGKRGYRL |
| NO: 9 | | HDERIAYSGGTGSCRSIKYELLNPDATRKNLLRGSGLQH |
| | | ELISAVRQDNLLLYGPLNFNDYIFDKDAPNLLHFWTLALS |
| | | LGFVFSNQNSIEREFKDYLGVSTEEAVLFGKLNETLKAV |
| | | FDEAKFISGFLYRNFRGLASKTREQRIKLLTDTLREPLDG |
| | | VNGDSVSEIIKPYAEKWAEYDGECDQFVFKCELFSIKST |
| | | DKPRENTRLSFAIDPAFEVMKLDDKTVFFDDLITHYKEN |
| | | CSDEAQAKRFLGIGDNGNYFNGIFGGLFELLTDGDEKIC |
| | | ETTDHLARIYGFDETKKTEINKRLVRLAEYARQINRRPCL |
| | | VKRWSEYRSDFNGTIESWYSNRQSKQNDTLKQLDEKLK |
| | | LLEEMRASFPTDSDLCGIKSLSETIEFIRSLKGERIARKVT |
| | | DELESYLAVLGSELNQYTQQNKDHALPLGWQKKLSKHI |
| | | QSSPLFFGENKIALWEKLINLKELIKTEVKELEVVLAEDF |
| | | DDYEITDKQVDNLAALAGRFSESPDGSGHPLVTERLAKI |
| | | ESTLGVDFTHKNNRAKFYLSGFERGKFGKLDVPNKIKVS |
| | | HLFELADLSILYNAVANSPEDGYILRDTAQLSKIILSAKL |
| | | RDADREKQRKTVLAHSTLQGYSALISKREFVSRYPLQAV |
| | | NGSQNLMAYDANRKYYYAYNSEKFAGTKELTVALRGN |
| | | NFGPEAFGGKFKKVPALRVQSSKYQIQFLDWFFEKQKK |
| | | RKTELGAGGSFTIAEISCKVNWDDKTPVIFEKPDPRLFVS |
| | | QPFTINPPENSAKKDYARYIGIDIGEYGLAWHLVEVFED |
| | | ANEDIGGAGKNAVRIKSVEKGFFTDPQQISLKEDVKKLR |
| | | ENQVRATFTSPDTKIARVRESLIGSYRNLLEDLAVRKDA |
| | | RLCFEYEVSGFESGGARISKVYDSIKRSSVAKKENKAEN |
| | | KQSWGKLFGPEFSFKAIEITAAGTSQYCTKCKRWASLAI |
| | | KDNNNYQLLEWDNGETGDKRGSDGLLAVTLDGEGKET |
| | | NRTVRLFPKDGKKAGDTIKGKDLKSAIYRAMRPNMRPS |
| | | EDGSISLGAGMEAVRRDLMPEQWEKLTLEFGQGKPRGN |
| | | MAIYVCPYCGHISDADMQAAFNIAVRGYLANRDKEKKV |
| | | KLGKEYLTDEQSKLTFDPVGILEHTT |
| SEQ ID | CasY15 | MPLMIRNTMNEKKTATQRRNARRRRGERARTKSQELRG |
| NO: 10 | | YRLHDARIEFSGGLGSMRTVKVELLNPDSSREDPQRGQG |
| | | LQGKVAKAVFDDYRALYGPMNIEDYLSDPDCPSFLGLW |
| | | VKAVCLGVIMSRKTATDFGELRGGSKSGQAFDSIPEHLR |
| | | RQLIKLKWLDWYDKGIRKSSSKASRLKSLTDVFANPKQP |
| | | DQGVMAAWEQGEKLAESSRDIAALGRREFKDKLFAIPPP |
| | | TSSVVLDDDVKATKVSRDWQWAVDPQFKLPSTDLDITR |
| | | ALEEVDRQWFERLGNNRGMVQQFFAIGDNGNHLNNGL |
| | | FGHFFASIRSANLADIVAEMGTAFGFSAEERDIVRQRLET |
| | | LHEYAQGLPEKPVLASRWAEYRTDMTAKLGSWYSNRT |
| | | SKGAASITQVWGTINTETGEVKDDGLVRTLENIQSDLPD |
| | | SCSIKEGILQETLDFIGDRRSSTDRAFTDELELYLATLRSD |
| | | LNTWCQEQSALWEEKQRQVATPASDEKSKKADNPWAG |
| | | KGSKTDKWLGALHTRIQSSPLFWGVDKLELWKTLANLK |
| | | QAIRDEIDKLNEQVEVFGRSAYDEPVGKDADSGEGDRR |
| | | VDQLSYLSARLGDQAHEEVRQRLDAIALALGVKFSERD |
| | | DLHRFFVSSRARRRAALLAMPNTITVGKLRELADLTPLW |
| | | ERIKKKPEEPRLLADTVALSKVVNSACASRANPSDQIELT |
| | | TIHSRLDGYSKNIGHTEFISRATVQSTNGAQNTVALDSLV |
| | | SPRLFYYNFPNIVESAEPHVSHLEVATRGNLGSFEEFAAK |
| | | EHRTFDRENPQKDSRNRIDSVNPLAVASSRYQIQFFTWW |
| | | AGLHRSKETALEVGGSFTIAERQVRLDWSQEKPQAVVS |
| | | EELRVFVSQPFTIVPDDKKRPATSGTRYIGVAIGEYGLA |
| | | WSCWEFAPGYWNGSVVNPSKVTCLDYGFLAEPGQRRIV |
| | | ERVKKLRESQATKTFTSPDTYIARLRENVVATYQAQLEA |
| | | LMMAYNAQLVFASEISAFETGGNRVKKIYDAIKRSSVFG |
| | | RSDAEATDNNQHWGKNGNRSSVKDPDKLRLNEAGQVA |
| | | ARVPWAEPVSAWMTSQTCSACGRVYVRAYRGKNSNEP |
| | | DSGATGEVRYFDNKQQKILTKTIGADTVWVTDQERKEF |
| | | ERGVYNAMRPNAFMPDGRWTAAGEILEAALKSRGTLD |
| | | GGRGFAGLHLTSKAQVHEYIEGTGKSHADAHGNSAIFIC |
| | | PYTDCGHIADAALQASYNIALRGFAYAIVRKKHPELFAG |
| | | SGSSTDGDEGGGKKPQQKQAFIDEIVRAAGRAS |
| SEQ ID | CasM.21524 | MKGYRLHDQRIAYSGGTGSMRSIKYELVDTDGSEGLRD |
| NO: 118 | | KVAGAIANDYRTLYGPLNFDDYLAGNRTPSLIDFWLKSL |
| | | SLGFVFSNQNSIESEFLEYLGKKTIWQNCYECLSDELKGV |
| | | VDEQAFCQFLIKSHRSVEKKTDEQRQKIILNLVKKGCDT |
| | | SALLPVAKDWSQKFSLDTDQLQLKCEIFGIPVPIVPQRDL |
| | | SLSFAVDPNFVVMDCSDRTEFLDQIIKFYEDKVGAAQAK |
| | | KFLAIGDNGNYFNGLFGNLLTCLKQGEVDSVAEFLDSTY |
| | | ELNNKVEISKRLAELKELADKIGEPELVNKWSDYRSDFN |
| | | GTIESWYSNRISKQQATLEQLDGKIDKKTGEVTGGLKEL |
| | | LKNISDALPEGNDIKEGILAETIAFLRGHGARIDRKFTDEL |
| | | ESYLATLKTDLNEWSQNNKEHKMPTGWQRELSKRVQS |
| | | SPLFFGENKYALWEQLIRLKGLIRDEVAKLEAVLQGQFE |
| | | DYAITDKQVDMLAQLAQRIDGDGNPEVIRRLADIERELQ |
| | | VNFGERSERARYFISGFERSKVTQLEIGNRINVSKLAELA |
| | | DLGELYDKLKNAPQDNYVLRDTAQLSKIVVSALVHGSD |
| | | KEREVVLMHSNLSGYASLISRREFITRCTVQAVNGGQLN |
| | | LGVRGNKYFYAFLPDKFDARSDVQLFSKTYNFTKADLK |
| | | DNTSSVPLLAVRSSKYQVQFLDWFMGRHSRKKTELGAG |
| | | GAFSIAEKTVKLDWSGETPRIAEISDPRVFVSQPFEIKPLG |
| | | KGTQASDNRFIGVDIGEYGLAWSLIEVNGNNVERLEDGF |
| | | IADLQQQKLKNAVKRLRESQVRATFGSPDTRVARIRESLI |
| | | GAYRNQLEDLAMRKNARLSFEYEVSGFEAGGARISKVY |
| | | DSIKRGDIRKKDNNAANKMAWGDFGVNNWGFETTAAG |
| | | TSQTCSKCRRWASLAIEDGKSYRLGEYQDKLFKAQIAD |
| | | GEVRLLAKQDTGETVKGKDLKGLIYKAMRPNDDGLGM |
| | | AIVKRQMDWDKLSKDFGAGKPRGNIAIFVCPYTDCHHI |
| | | ADADLQAALNIAIRGYGKRKSDGKMGKVNDFAEFTKEL |
| | | QYDPVGFAS |
| SEQ ID | CasM.21518 | MNKKSSNSTGYRLHKDRILFSGGEIMRTIKYPLVVEKNN |
| NO: 119 | | LNSEEIVEKIRQAIINDDRVIRSDINLNDYIEYTKKGNRLY |
| | | TLIDFWQDCLRAGVIWQPSTSFLLYLINKLYSKPKAIELIE |
| | | NAKPDISRFFDVDKFSKCFILPGEIREGKILKTFKRELIEAL |
| | | KGEFKKGKKEKIKDEDDYLEKFVEKDARKLIREIADCFF |
| | | SNDILVTHDLKEGKKEYQDRLWEEKFGIKKGKLLENFK |
| | | LPDHLRNFKNISFFIIPELSDKSKNFDELIELRRKWLLERKI |
| | | CVREDGDYLENEKKLDEELRNLVGLSDNCNPLSNFLGT |
| | | VFCELLVPNNLNEDNALEKFYDVFTIVEPKIAELNIKDQI |
| | | MGSLEFLRLRAKQLGSPNLVNFSKSQNLKANESIKLDG |
| | | WSLYRQNFGSKMQSWFTSYIERNKLLEDSLKNFKEKIKK |
| | | AQNFIKNLKNISEEPQQEEEAQQEKEEIVELFEKIFSSLEK |
| | | VNRENFEVFDSLLSSLRKRLNFFYQQYLYNEAKEGDDV |
| | | KKHKILGPIFKNIEKPIAFYGETQRKKNEKFVEDTIPILEE |
| | | GTVFLTTLISNLLDSFSPKQVFPDVRKKDETEEIIYRKELQ |
| | | FFWNKLKDLAVNSKEFEKEYQDIIESAVDESELSKLKELF |
| | | VNKKKNGSKYNKYTFYKSKYTKGSIEEIKLKGSKEEYLL |
| | | RFEKLIKSLTNFLTQFNRNKLLQDKDLLLDWVELAKNIV |
| | | SVLIRFSTNTEFSLNEIKAQSQFKKAKNYLELFKLKKAKK |
| | | KEFGFIIQSFILSEIKGAATLYSKRKYIASYSVQIVGSNNK |
| | | FKLFYQPLDSSINISGGPKDFVTKKHKYLIVFQDLKNVKN |
| | | KDATENRINLLRLNKERKIPLVAYKDDLVSKSLLLSSSPY |
| | | QLQFLDKYLYRPRGWENIDIKLNEWSFVVEEAYDIEWD |
| | | LNSKTPKLIPSPKSNRNKLYLAIPFTLKGNVKEPPLDKIVL |
| | | KSETKKDHSRDKNRLNYPILGVDVGEYGVAWCLTKFDY |
| | | NQDFSLRDIDIQGKGFIEDRNIGKIKDYFAEIQQKSRKGA |
| | | YDEDDTTIAKVRENAIGKLRNAIHSILTGSLEGASPVYED |
| | | AISNFETGSGKTIKIYNSVKRADTEFKSEADKAEHSLVW |
| | | GKKDRNQETKYIGRNVSAYASSYTCVNCLHTLFKVKKE |
| | | DLSNIKILEKDGRIVTMSSPYGPDKKVRGYLSEKEKYEIG |
| | | YQFKESEEDLKAFRKIVRDFARPPVNKNSEVLEKYAKEI |
| | | LAGNKIEEFRKKRGNSAIFVCPFCQFKADADIQAAFMMA |
| | | MRGYLRFSGIVPSKENSKNNPQESEDKSLKNSKKQSETG |
| | | DTFLTKTAEYLQQLRFEIKEKIKEAVKVDF |
| SEQ ID | CasM.21520 | MNEKKTATQRRNARRRRGERARTKSQELRGYRLHDARI |
| NO: 120 | | EFSGGLGSMRTVKVELLNPDSSREDPQRGQGLQGKVAK |
| | | AVFDDYRALYGPMNIEDYLSDPDCPSFLGLWVKAVCLG |
| | | VIMSRKTATDFGELRGGSKSGQAFDSIPEHLRRQLIKLK |
| | | WLDWYDKGIRKSSSKASRLKSLTDVFANPKQPDQGVM |
| | | AAWEQGEKLAESSRDIAALGRREFKDKLFAIPPPTSSVVL |
| | | DDDVKATKVSRDWQWAVDPQFKLPSTDLDITRALEEVD |
| | | RQWFERLGNNRGMVQQFFAIGDNGNHLNNGLFGHFFAS |
| | | IRSANLADIVAEMGTAFGFSAEERDIVRQRLETLHEYAQ |
| | | GLPEKPVLASRWAEYRTDMTAKLGSWYSNRTSKGAASI |
| | | TQVWGTINTETGEVKDDGLVRTLENIQSDLPDSCSIKEGI |
| | | LQETLDFIGDRRSSTDRAFTDELELYLATLRSDLNTWCQ |
| | | EQSALWEEKQRQVATPASDEKSKKADNPWAGKGSKTD |
| | | KWLGALHTRIQSSPLFWGVDKLELWKTLANLKQAIRDEI |
| | | DKLNEQVEVFGRSAYDEPVGKDADSGEGDRRVDQLSYL |
| | | SARLGDQAHEEVRQRLDAIALALGVKFSERDDLHRFFVS |
| | | SRARRRAALLAMPNTITVGKLRELADLTPLWERIKKKPE |
| | | EPRLLADTVALSKVVNSACASRANPSDQIELTTIHSRLDG |
| | | YSKNIGHTEFISRATVQSTNGAQNTVALDSLVSPRLFYY |
| | | NFPNIVESAEPHVSHLEVATRGNLGSFEEFAAKEHRTFD |
| | | RENPQKDSRNRIDSVNPLAVASSRYQIQFFTWWAGLHRS |
| | | KETALEVGGSFTIAERQVRLDWSQEKPQAVVSEELRVFV |
| | | SQPFTIVPDDKKRPATSGTRYIGVDIGEYGLAWSCWEFA |
| | | PGYWNGSVVNPSKVTCLDYGFLAEPGQRRIVERVKKLR |
| | | ESQATKTFTSPDTYIARLRENVVATYQAQLEALMMAYN |
| | | AQLVFESEISAFETGGNRVKKIYDAIKRSSVFGRSDAEAT |
| | | DNNQHWGKNGNRSSVKDPDKLRLNEAGQVAARVPWA |
| | | EPVSAWMTSQTCSACGRVYVRAYRGKNSNEPDSGATG |
| | | EVRYFDNKQQKILTKTIGADTVWVTDQERKEFERGVYN |
| | | AMRPNAFMPDGRWTAAGEILEAALKSRGTLDGGRGFA |
| | | GLHLTSKAQVHEYIEGTGKSHRDAHGNSAIFICPYTDCG |
| | | HIADADLQASYNIALRGFAYAIVRKKHPELFAGSGSSTD |
| | | GDEGGGKKPQQKQAFIDEIVRAAGRAS |
| SEQ ID | CasM.21522 | NPLGITACLLPGFETPDAYRAGRDVTLLYHVQRMQRLLS |
| NO: 121 | | LDEVKEAYEFVGMHDSALSNFLNGNSNKGFLALLLRGE |
| | | FDTLARGMMDMTPLWNEHNHDVLMNRLQALGRNAQK |
| | | LSFTKPRFGNSFADHRKQISGKATAWFSGYCNKLDIAKE |
| | | QIPLVLEDAKMFMEMLCAVEIDYEKEEFMHLQLSAFIER |
| | | MERAHERLDADGVVALKKAQYVLPIIREFANPIVQREEA |
| | | QQWLSLNIELVPDKRFTFKQAFPYLSDQGGADTEKGEQT |
| | | KFQSVPRFFAEGAHAEYVAFSQAPMIFSVYMENLRLLW |
| | | DRLIGMERRTPRNWEEHLSKLLYYLYVDIYVECRTDAC |
| | | KKYVRGILETYAHVSRIPEHVPGIKRFTIKPGYTPQGGLT |
| | | KDQLVQCVLQIAQRLSNAGFEWKRISALERLDLVEVHK |
| | | KAFAFLVGITHGSVDVSAYNWLNNKSVVQYLDVLKTTD |
| | | LGGIRLARFLQSCVCATLRGSATRMSEQMLTARFSVQTA |
| | | TTIPQCELVYAVSEYMRKRRFVTPPSHVSRDVLDRKPASI |
| | | VAQAIYNPEQMAGSMRFIPHRFGYQIQLPELARHESLNN |
| | | ALVVQKPTSTRRYSFARFDAEKGPVLWVESSHYQQQYF |
| | | DWFFFAPNNRPADVIPQGVSLVVEQDIALQWDFDALVV |
| | | HMQPVHEPRMYCPQPFKFVPRVESSVMQNRYMGIAPGT |
| | | NEVAYAVIEVNGTRVSVINCGTFPLAGFANLSRAEEKKQ |
| | | KRERRVGSHAFSPDKRAIVLDNSANKLANQIHACAIQNR |
| | | ARLVWQWTPQTVQIAKGREVDMVYARARKSCAPKNDS |
| | | DFDAMKTKWGKIWSAKWEDGKHTLSTEVYYAESLFQS |
| | | ACNAPVPPECATALMVALLGRVRDIATPKKWEDDAWR |
| | | ADTLAELFQQITH |
| SEQ ID | CasM.21516 | MFNQKKGYRLHLERIIYSGGEITRSIKYLLASHSDSQKNK |
| NO: 122 | | ELLNNFSQDLYNDDLKIRGCLNLNDLVNNNQIYNLADF |
| | | WIDSLRAGVIWQSSASSLIDFIKRLNHQETIGEKIFNNANE |
| | | RIKRFFNSEKFIKEIILSEPKRISSKKQAFYNSLFDILKDEF |
| | | KKQEKNEKIIIDNKAEQLIKEIVDAFYSNDGVFLMEGEEK |
| | | QNNFWQEKFNIDKNMIKKEKEDILKDVGDITAFIHPPLIIL |
| | | KGDVSQLIDERKKYFSEKDLEEILGLSDNFNAFSHYFNKF |
| | | FLLLYQDKQEKIFECYQKIFSFSQEDRKRIKDALDFLLEK |
| | | SKLLGLPKIVNSWSDYRSVFGGKIKSWFSNYLNREDKAK |
| | | KQEKKIKEGLEKVNKFLLDFIQKNQVDSDLQQEIKFYYD |
| | | KLNQFINSYQNQEFFHQQELFLLFSDLLAEYREKLNRFY |
| | | QKYLSDKEKEEKKVDEFPLFKDLFEKYEGPISFYGKTKL |
| | | EDNKKIIDLTFKTIKVGLNLIRRLLIDLYNSSDFKNSDNN |
| | | NQERDLRRIFEFLLNKIPATKTFREKYLSILKDNFDQQTY |
| | | KEMTLKPSRYTFVENIYSRENRKLIELPSKNFEELLSKIIK |
| | | DLTDFSLSFKNDDLFVDIYLLSDLVELAKTLISLVINYSN |
| | | KSQFDSYKNELIDDTYQKAKKYLETFKISFFNSKKEANY |
| | | FYQTRVLSELKGAVALFSKKYYQAKYNIQILKSNEIFPLF |
| | | VKFSDLLKKEEINDINKLKLIFKKPYRYLIALKKIKFKKK |
| | | QQQSSVIHLDKKNKDLVLISPQDEDFLFKLTSSFYQLQFL |
| | | DRFVYPVKKWLNVDITLSEWSFILEKKYKINWDFNNGK |
| | | PEFSEIDSRLYLNIPFKIKAINQQKILKPKELFLGIDVGEYG |
| | | VGYALVNFKDEEIKIIKSGFIRSKNIASIRDKYRLLQDRSK |
| | | KGVYFSSTNVVQEVRENAIGEIRNQIHDILIKNNADLIYE |
| | | YNISNFETGSGRITKIYDSIKKSDVYAENEADKSVIQHVW |
| | | GIKKSIASHLSAYGSSYTCSNCGRSIFSFSENDIFSSKVIKR |
| | | DGNIITIQTPKGEVFAYSKDKKFNIGYSFSQEKNKEEMKN |
| | | LFMKIVKAYARPPLLKSEVLLTQKKLDREFLEKFKKERG |
| | | NSAIFVCPFVDCQSLADSDIQAAFIMALRGYLKKKKGKD |
| | | INYLEESLNYLQNFKGKINFSNLLH |
| SEQ ID | CasM.21466 | MAESKQMQCRKCGASMKYEVIGLGKKSCRYMCPDCGN |
| NO: 123 | | HTSARKIQNKKKRDKKYGSASKAQSQRIAVAGALYPDK |
| | | KVQTIKTYKYPADLNGEVHDRGVAEKIEQAIQEDEIGLL |
| | | GPSSEYACWIASQKQSEPYSVVDFWFDAVCAGGVFAYS |
| | | GARLLSTVLQLSGEESVLRAALASSPFVDDINLAQAEKF |
| | | LAVSRRTGQDKLGKRIGECFAEGRLEALGIKDRMREFVQ |
| | | AIDVAQTAGQRFAAKLKIFGISQMPEAKQWNNDSGLTV |
| | | CILPDYYVPEENRADQLVVLLRRLREIAYCMGIEDEAGF |
| | | EHLGIDPGALSNFSNGNPKRGFLGRLLNNDIIALANNMS |
| | | AMTPYWEGRKGELIERLAWLKHRAEGLYLKEPHFGNS |
| | | WADHRSRIFSRIAGWLSGCAGKLKIAKDQISGVRTDLFL |
| | | LKRLLDAVPQSAPSPDFIASISALDRFLEAAESSQDPAEQ |
| | | VRALYAFHLNAPAVRSIANKAVQRSDSQEWLIKELDAV |
| | | DHLEFNKAFPFFSDTGKKKKKGANSNGAPSEEEYTETES |
| | | IQQPEDAEQEVNGQEGNGASKNQKKFQRIPRFFGEGSRS |
| | | EYRILTEAPQYFDMFCNNMRAIFMQLESQPRKAPRDFKC |
| | | FLQNRLQKLYKQTFLNARSNKCRALLESVLISWGEFYTY |
| | | GANEKKFRLRHEASERSSDPDYVVQQALEIARRLFLFGF |
| | | EWRDCSAGERVDLVEIHKKAISFLLAITQAEVSVGSYNW |
| | | LGNSTVSRYLSVAGTDTLYGTQLEEFLNATVLSQMRGL |
| | | AIRLSSQELKDGFDVQLESSCQDNLQHLLVYRASRDLAA |
| | | CKRATCPAELDPKILVLPAGAFIASVMKMIERGDEPLAG |
| | | AYLRHRPHSFGWQIRVRGVAEVGMDQGTALAFQKPTES |
| | | EPFKIKPFSAQYGPVLWLNSSSYSQSQYLDGFLSQPKNW |
| | | SMRVLPQAGSVRVEQRVALIWNLQAGKMRLERSGARA |
| | | FFMPVPFSFRPSGSGDEAVLAPNRYLGLFPHSGGIEYAVV |
| | | DVLDSAGFKILERGTIAVNGFSQKRGERQEEAHREKQRR |
| | | GISDIGRKKPVQAEVDAANELHRKYTDVATRLGCRIVV |
| | | QWAPQPKPGTAPTAQTVYARAVRTEAPRSGNQEDHAR |
| | | MKSSWGYTWGTYWEKRKPEDILGISTQVYWTGGIGESC |
| | | PAVAVALLGHIRATSTQTEWEKEEVVFGRLKKFFPS |

In some embodiments, compositions and methods described herein comprise a programmable nuclease comprising or consisting of an amino acid sequence that is at least 70%, at least 75%, at least 80%, at least 85%, at least 90%, at least 92%, at least 95%, at least 97%, at least 98%, at least 99%, or 100% identical to any one SEQ ID NOs: 1-10 and SEQ ID NOs: 118-123. In some embodiments, compositions and methods described herein comprise a programmable nuclease comprising or consisting of an amino acid sequence that is at least 70%, at least 75%, at least 80%, at least 85%, at least 90%, at least 92%, at least 95%, at least 97%, at least 98%, at least 99%, or 100% identical to any one SEQ ID NOs: 1-10. In some embodiments, compositions and methods described herein comprise a programmable nuclease comprising or consisting of an amino acid sequence that is at least 70%, at least 75%, at least 80%, at least 85%, at least 90%, at least 92%, at least 95%, at least 97%, at least 98%, at least 99%, or 100% identical to any one SEQ ID NOS: 118-123.

In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 1. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 2. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 3. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 4. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 5. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 6. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 7. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 8. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 9. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 10. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 118. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 119. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 120. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 121. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 122. In some instances, the programmable nuclease comprises or consists of an amino acid sequence that is at least 70%, at least 75%, at least 80%, at least 85%, at least 90%, at least 92%, at least 95%, at least 97%, at least 98%, at least 99%, or 100% identical to SEQ ID NO: 123.

The programmable nuclease can be a CRISPR/Cas (clustered regularly interspaced short palindromic repeats—CRISPR associated) ribonucleoprotein (RNP) complex with trans cleavage activity, which can be activated by binding of the spacer a crRNA to a target nucleic acid. The programmable nuclease can be a CRISPR/Cas (clustered regularly interspaced short palindromic repeats−CRISPR associated) nucleoprotein complex with cis cleavage activity, which can be activated by binding of the spacer of a crRNA to a target nucleic acid. The CRISPR/Cas ribonucleoprotein (RNP) complex can comprise a Cas protein complexed with an engineered guide RNA (egRNA) comprising a crRNA and an intermediary nucleic acid. Sometimes, the crRNA and the intermediary nucleic acid are engineered as a single polyribonucleotide, referred to herein as a composite egRNA. An assay using the CRISPR/Cas RNP complex to detect target nucleic acids can comprise crRNAs, intermediary RNAs, Cas proteins, and detector nucleic acids. The CRISPR/Cas RNP complex used to modify target nucleic acids can comprise crRNAs, intermediary RNAs, Cas proteins, and target nucleic acids in a sample from a subject.

The programmable nucleases (e.g., a CasY protein) described herein may be activated to exhibit cleavage activity (e.g., cis cleavage of a target nucleic acid or trans cleavage of a collateral nucleic acid) upon binding of a ribonucleoprotein (RNP) (a complex of a programmable nuclease and egRNA system comprising the intermediary RNA and a crRNA) to a target nucleic acid (e.g., DNA), in which the spacer of the crRNA hybridizes to the target nucleic acid. Once activated, the programmable nuclease may specifically cleave the target nucleic acid. The programmable nuclease may have cis cleavage activity once activated. Once activated, the programmable nuclease may non-specifically degrade nucleic acids in its environment. The programmable nuclease may have trans cleavage activity once activated.

In some cases, the programmable nuclease is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pini), Enterococcus italicus (Ei), Lactobacillus salivarius (Ls), or Therms thermophilus (Tt). Sometimes the programmable nuclease is a CasY protein.

### 2. Applications

The programmable nucleases (e.g., CasY proteins), egRNA systems, and methods of use thereof disclosed herein may be applied to a variety of assays, techniques, and procedures including agricultural, biochemical, biomedical, diagnostic, and genetic engineering applications. In some embodiments, the programmable nucleases, egRNA systems, and methods of use thereof disclosed herein may be used to modify a target nucleic acid. In some embodiments, modification of a target nucleic acid comprising a region of a genome may be referred to herein as genome editing. The target nucleic acid may be from an animal or a plant. In some embodiments, the programmable nucleases, egRNA systems, and methods of use thereof disclosed herein may be used to detect the presence or absence of a target nucleic acid in a sample. For example, the programmable nucleases, egRNA systems, and methods of use thereof disclosed herein may be used to detect the presence or absence of a target nucleic acid associated with a disease or condition, there by diagnosing the disease or condition. Additionally, or alternatively, the programmable nucleases, egRNA systems, and methods of use thereof disclosed herein may be used to quantify the amount of the target nucleic acid associated with a disease or condition that is present in a sample.

a. Genome Editing

The programmable nucleases and egRNA systems disclosed herein may be used to modify a target nucleic acid. Described herein are methods of modifying a target nucleic acid using compositions comprising a programmable nuclease (e.g., a CasY protein) and an egRNA system (e.g., a discrete egRNA system or a composite egRNA). Modifying a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid. The target nucleic acid may comprise one or more of a genome, a chromosome, a plasmid, a gene, a promoter, an untranslated region, an open reading frame, an intron, an exon, or an operator. The target nucleic acid may comprise a segment of one or more of a genome, a chromosome, a plasmid, a gene, a promoter, an untranslated region, an open reading frame, an intron, an exon, or an operator. In some embodiments, the target nucleic acid may be part of a cell or an organism. In some embodiments, the target nucleic acid may be a cell-free genetic component. In some embodiments, modifying a target nucleic acid comprises genome editing. Genome editing may comprise modifying a genome, chromosome, plasmid, or other genetic material of a cell or organism. In some embodiments the genome, chromosome, plasmid, or other genetic material of the cell or organism is modified in vivo. In some embodiments the genome, chromosome, plasmid, or other genetic material of the cell or organism is modified in a cell. In some embodiments the genome, chromosome, plasmid, or other genetic material of the cell or organism is modified in vitro. For example, a plasmid may be modified in vitro using a composition described herein and introduced into a cell or organism.

Editing of Eukaryotic Cells. The methods, systems, and compositions disclosed herein may be used to edit eukaryotic cells. Eukaryotic genome editing, as disclosed herein, may be used to may be used to generate targeted gene mutations, treat or prevent genetic diseases or conditions, create chromosome rearrangements, study gene function, reprogram stem cells, endogenously label genes, or create targeted transgene additions in one or more eukaryotic cells. In some embodiments, eukaryotic genome editing may be used to repair one or more mutations associated with a disease or condition or replace a gene comprising one or more mutations associated with a disease or condition with a functional gene (e.g., a gene lacking mutations associated with a disease or condition), thereby treating or preventing the disease or condition. Repair or replacement of a gene comprising one or more mutations associated with a disease or a condition may be referred to herein as gene therapy. Gene therapy may comprise modification of a reproductive cell (e.g., a sperm cell or an egg cell), also referred to as germline gene therapy. Alternatively, or in addition, gene therapy may comprise modification of a somatic cell (e.g., a cell within a multicellular organism), also referred to as somatic cell gene therapy.

In some embodiments, eukaryotic genome editing may be used to modify the genome of a stem cell. A genetically modified stem cell may be introduced into an organism (e.g., a human) to treat a disease or a condition. Introduction of a stem cell (e.g., a genetically modified stem cell) into an organism to treat a disease or condition may be referred to herein as stem cell therapy. For example, a genetically modified stem cell may replace or repair damaged tissue associated with spinal cord injury, type 1 diabetes, Parkinson's disease, amyotrophic lateral sclerosis (ALS), Alzheimer's disease, heart disease, stroke, burn, cancer, or osteoarthritis.

Methods of editing a eukaryotic cell may comprise contacting a eukaryotic cell comprising a target nucleic acid to a programmable nuclease or a polynucleotide encoding a programmable nuclease, contacting the eukaryotic cell to an RNA component or a polynucleotide encoding the RNA component, and modifying the target nucleic acid. Alternatively, or in addition, methods of editing a eukaryotic cell may comprise contacting a target nucleic acid to a programmable nuclease and an RNA component, modifying the target nucleic acid, and contacting the modified target nucleic acid to a eukaryotic cell. The target nucleic acid may comprise a genome, a chromosome, a plasmid, a gene, a promoter, an untranslated region, an open reading frame, an intron, an exon, or an operator of a eukaryotic cell, or the target nucleic acid may comprise a segment of a genome, a chromosome, a plasmid, a gene, a promoter, an untranslated region, an open reading frame, an intron, an exon, or an operator of a eukaryotic cell. The programmable nuclease may be a Cas12 programmable nuclease (e.g., a CasY protein), as described herein. The RNA component may be a discrete egRNA, or the RNA component may be a composite egRNA. The RNA component may comprise a crRNA and an intermediary RNA. Modifying the target nucleic acid may comprise contacting the target nucleic acid with a complex comprising a programmable nuclease, a crRNA that hybridizes to a region of the target nucleic acid, and an intermediary RNA; activating target cleavage activity of the programmable nuclease; and introducing one or more double stranded breaks into the target nucleic acid. In some embodiments, modifying the target nucleic acid may comprise removing a segment of the target nucleic acid between a first double stranded break and a second double stranded break, thereby deleting the segment of the target nucleic acid. In some embodiments, modifying the target nucleic acid may comprise removing a segment of the target nucleic acid between a first double stranded break and a second double stranded break and inserting a donor nucleic acid between the first double stranded break and the second double stranded break, thereby replacing the segment of the target nucleic acid with the donor nucleic acid. In some embodiments, modifying the target nucleic acid may comprise inserting a donor nucleic acid at a double stranded break, thereby inserting the donor nucleic acid into the target nucleic acid.

A eukaryotic cell comprising a modified target nucleic acid may be a transgenic cell or a genetically modified cell. An organism comprising a transgenic cell may be a transgenic organism or a genetically modified organism. In some embodiments, a transgenic cell may have one or more of an altered gene expression, an altered gene product, or an altered phenotype relative to a non-transgenic cell. Editing a eukaryotic cell may comprise modifying a chromosome of a eukaryotic genome. In some embodiments, editing a eukaryotic cell may comprise modifying a plasmid of a eukaryotic cell. In some embodiments, editing a eukaryotic cell may comprise modifying an organelle genome (e.g., a mitochondrial genome) of a eukaryotic cell. In some embodiments, the chromosome, plasmid, or organelle genome is modified in the eukaryotic cell, thereby producing a transgenic eukaryotic cell. Alternatively or in addition, the chromosome, plasmid, or organelle genome is modified in vitro and the modified chromosome, plasmid, or organelle genome is introduced into the eukaryotic cell, thereby producing a transgenic eukaryotic cell. A eukaryotic cell may be modified in vivo (e.g., in an organism) or ex vivo (e.g., in cell culture).

In some embodiments, the eukaryotic cell may be a unicellular organism. For example, the eukaryotic cell may be a protozoon, a unicellular alga, or a unicellular fungus (e.g., a yeast). In some embodiments, the eukaryotic cell may be in a multicellular organism. For example, the eukaryotic cell may be in an animal (e.g., a human), a plant, a multicellular alga, or a multicellular fungus. In some embodiments, the eukaryotic cell may be a cultured cell. For example, the eukaryotic cell may be a cultured stem cell (e.g., an adult stem cell, a fetal stem cell, a pluripotent stem cell, or a reprogrammed stem cell), a cultured mammalian cell (e.g., a HeLa cell, a CHO cell, or a COS cell), a cultured insect cell (e.g., an SF9 cell), a cultured plant cell, or a cultured fungal cell (e.g., a yeast culture cell). In some embodiments, the eukaryotic cell may be a germline cell. For example, the eukaryotic cell may be a sperm, an egg, or a spore. As described herein, the methods of modifying a target nucleic acid in a eukaryotic cell may be used to treat or prevent a genetic disease or condition, for example by deleting, replacing, modifying, or inserting a gene associated with the genetic disease or condition. In some embodiments, the genetic disease or condition may be Huntington's disease, neurofibromatosis type 1, neurofibromatosis type 2, Marfan syndrome, hereditary nonpolyposis colorectal cancer, hereditary multiple exostoses, tuberous sclerosis, Von Willebrand disease, acute intermittent porphyria, albinism, medium-chain acyl-CoA dehydrogenase deficiency, cystic fibrosis, sickle cell disease, Tay-Sachs disease, Niemann-Pick disease, spinal muscular atrophy, Roberts syndrome, familial hypercholesterolemia, polycystic kidney disease, hereditary spherocytosis, phenylketonuria, mucopolysaccharidosis, lysosomal acid lipase deficiency, glycogen storage diseases, galactosemia, Duchenne muscular dystrophy, hemophilia, thalassaemia, or Leber's hereditary optic neuropathy, myotonic dystrophy Type 1 (DM1), oncology diseases, ophthalmology diseases, inherited diseases of the back of the eye, and cystic fibrosis.

The sample used for cancer testing may comprise at least one target nucleic acid that can bind to a guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay can be used to detect “hotspots” in target nucleic acids that can be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. Any region of the aforementioned gene loci can be probed for a mutation or deletion using the compositions and methods disclosed herein. For example, in the EGFR gene locus, the compositions and methods for detection disclosed herein can be used to detect a single nucleotide polymorphism or a deletion. The SNP or deletion can occur in a non-coding region or a coding region.

The sample used for genetic disorder testing may comprise at least one target nucleic acid that can bind to a guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that can bind to a guide nucleic acid of the reagents described herein. The target nucleic acid, in some cases, is a nucleic acid encoding a sequence associated with a phenotypic trait.

The sample used for genotyping testing may comprise at least one target nucleic acid that can bind to a guide nucleic acid of the reagents described herein. The target nucleic acid, in some cases, is a nucleic acid encoding a sequence associated with a genotype of interest.

The sample used for ancestral testing may comprise at least one target nucleic acid that can bind to a guide nucleic acid of the reagents described herein. The target nucleic acid, in some cases, is a nucleic acid encoding a sequence associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status, but the status of any disease can be assessed.

Bioproduction. The methods, systems, and compositions disclosed herein may be used to introduce an exogenous gene into a cell for bioproduction. The exogenous gene may be a transgene, an artificial gene, an engineered gene, a modified transgene. Alternatively, or in addition, the methods, systems, and compositions disclosed herein may be used to modify an endogenous gene in a cell for bioproduction. Modifying an endogenous gene may comprise modifying the coding sequence, modifying the non-coding sequence, altering gene expression, truncating the gene, or creating a gene fusion. A cell comprising the exogenous gene, or the modified endogenous gene may be referred to herein as a modified cell. The modified cell may express the exogenous gene or the modified endogenous gene to produce an exogenous gene product. For example, the exogenous gene product may be a biological product, a protein, a peptide, oligonucleotide, a DNA, or an RNA. In some embodiments, the exogenous gene product may produce an exogenous reaction product. For example, an exogenous protein may catalyze production of a biological product, a small molecule, or a polymer. Production of an exogenous gene product or an exogenous reaction product by a modified cell may be referred to herein as bioproduction. Bioproduction, as disclosed herein, may comprise production of a biological product. For example, bioproduction may comprise production of a biologic-based pharmaceutical, a biofuel, an enzymatic reaction product, an amino acid, an engineered protein, an antibody, an enzyme, a detergent, or a polymer (e.g., a plastic). In some embodiments, bioproduction comprise facilitating a reaction to treat, remove, or degrade an environmental pollutant (e.g., bioremediation). For example, bioproduction may comprise expressing an enzyme to sequester carbon dioxide, oxidize hydrocarbons, or reduce nitrates, perchlorates, oxidized metals, chlorinated solvents, explosives or propellants.

Methods of gene editing for bioproduction may comprise contacting a cell comprising a target nucleic acid to a programmable nuclease or a polynucleotide encoding a programmable nuclease, contacting the cell to an RNA component or a polynucleotide encoding the RNA component, and modifying the target nucleic acid. Alternatively, or in addition, methods of editing a cell for bioproduction may comprise contacting a target nucleic acid to a programmable nuclease and an RNA component, modifying the target nucleic acid, and contacting the modified target nucleic acid to a cell. The target nucleic acid may comprise a genome, a chromosome, a plasmid, a gene, a promoter, an untranslated region, an open reading frame, an intron, an exon, or an operator, or the target nucleic acid may comprise a segment of a genome, a chromosome, a plasmid, a gene, a promoter, an untranslated region, an open reading frame, an intron, an exon, or an operator. The programmable nuclease may be a Cas12 programmable nuclease (e.g., a CasY protein), as described herein. The RNA component may be a discrete egRNA system, or the RNA component may be a composite egRNA. The RNA component may comprise a crRNA and an intermediary RNA. Modifying the target nucleic acid may comprise contacting the target nucleic acid with a complex comprising a programmable nuclease, a crRNA that hybridizes to a region of the target nucleic acid, and an intermediary RNA; activating target cleavage activity of the programmable nuclease; and introducing one or more double stranded breaks into the target nucleic acid. In some embodiments, modifying the target nucleic acid may comprise removing a segment of the target nucleic acid between a first double stranded break and a second double stranded break, thereby deleting the segment of the target nucleic acid. In some embodiments, modifying the target nucleic acid may comprise removing a segment of the target nucleic acid between a first double stranded break and a second double stranded break and inserting a donor nucleic acid (e.g., an exogenous gene or a modified endogenous gene) between the first double stranded break and the second double stranded break, thereby replacing the segment of the target nucleic acid with the donor nucleic acid. In some embodiments, modifying the target nucleic acid may comprise inserting a donor nucleic acid (e.g., an exogenous gene or a modified endogenous gene) at a double stranded break, thereby inserting the donor nucleic acid into the target nucleic acid.

In some embodiments, a modified cell may have one or more of an altered gene expression, an altered gene product, or an altered phenotype relative to an unmodified cell. Editing a cell for bioproduction may comprise modifying a chromosome of a cellular genome. In some embodiments, editing a cell for bioproduction may comprise modifying a plasmid of a cell. In some embodiments, editing a cell for bioproduction may comprise modifying an organelle genome (e.g., a mitochondrial genome) of a cell. In some embodiments, the chromosome, plasmid, or organelle genome is modified in the cell, thereby producing a modified cell. Alternatively, or in addition, the chromosome, plasmid, or organelle genome is modified in vitro and the modified chromosome, plasmid, or organelle genome is introduced into the cell, thereby producing a modified cell.

A modified cell comprising an exogenous gene, or a modified endogenous gene may be a unicellular organism, a cultured cell, a biofilm, an alga, or a fungus. A modified cell expressing an exogenous gene product may be a unicellular organism, a cultured cell, a biofilm, an alga, or a fungus. A modified cell producing an exogenous reaction product may be a unicellular organism, a cultured cell, a biofilm, an alga, or a fungus. Unicellular organisms that may be modified using the methods, systems, and compositions disclosed herein may include bacteria, yeast, unicellular algae, protists, archaea, and protozoa. Cultured cells that may be modified using the methods, systems, and compositions disclosed herein may include cultured mammalian cells, cultured stem cells, yeast, cultured insect cells, or cultured plant cells.

As described herein, the methods of modifying a target nucleic acid in a cell for bioproduction may be used to produce an exogenous gene product or an exogenous reaction product. In some embodiments, the methods of modifying a target nucleic acid in a cell for bioproduction may be used to produce a biological product (e.g., a peptide, a protein, or an enzymatic reaction product). For example, bioproduction may include production of a biologic drug (e.g., a peptide drug) encoded by an exogenous gene or a modified endogenous gene in a genetically modified cell. In another example, bioproduction may include production of a biofuel enzymatically synthesized by a protein encoded by an exogenous gene or a modified endogenous gene in a genetically modified cell. Alternatively, or in addition, the methods of modifying a target nucleic acid in a cell for bioproduction may be used to facilitate a reaction to treat, remove, or degrade an environmental pollutant (e.g., bioremediation). For example, bioproduction may include enzymatic degradation of a pollutant by a protein encoded by an exogenous gene or a modified endogenous gene in a genetically modified cell.

Compositions and methods of the disclosure can be used for cell line engineering (e.g., engineering a cell from a cell line for bioproduction). For example, compositions and methods of the disclosure can be used to express a desired protein from a cell line. In some embodiments, the target nucleic acid sequence comprises a nucleic acid sequence of a cell line. In some embodiments, the target nucleic acid sequence comprises a genomic nucleic acid sequence of a cell line. In some embodiments, the cell line is a Chinese hamster ovary cell line (CHO), human embryonic kidney cell line (HEK), cell lines derived from cancer cells, cell lines derived from lymphocytes, and the like. Non-limiting examples of cell lines includes: C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR. Non-limiting examples of other cells that can be used with the disclosure include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells. Non-limiting examples of cells that can be used with this disclosure also include plant cells, such as parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that can be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

Methods of the disclosure can be performed in a subject. Compositions of the disclosure can be administered to a subject. A subject can be a human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician). A subject can be a plant or a crop.

Methods of the disclosure can be performed in a cell. A cell can be in vitro. A cell can be in vivo. A cell can be ex vivo. A cell can be an isolated cell. A cell can be a cell inside of an organism. A cell can be an organism. A cell can be a cell in a cell culture. A cell can be one of a collection of cells. A cell can be a mammalian cell or derived from a mammalian cell. A cell can be a rodent cell or derived from a rodent cell. A cell can be a human cell or derived from a human cell. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a pluripotent stem cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a microbe cell or derived from a microbe cell. A cell can be a fungi cell or derived from a fungi cell. A cell can be from a specific organ or tissue.

Methods of the disclosure can be performed in a eukaryotic cell or cell line. In some embodiments, the eukaryotic cell is a Chinese hamster ovary (CHO) cell. In some embodiments, the eukaryotic cell is a Human embryonic kidney 293 cells (also referred to as HEK or HEK 293) cell.

Non-limiting examples of cell lines that can be used with the disclosure include C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR. Non-limiting examples of other cells that can be used with the disclosure include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells. Non-limiting examples of cells that can be used with this disclosure also include plant cells, such as Parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that can be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

Editing of Plants. The methods, systems, and compositions disclosed herein may be used to edit plant cells. Plant genome editing, as disclosed herein, may be used to may be used to generate targeted gene mutations, introduce desired traits, introduce or modify genes for bioproduction, create chromosome rearrangements, study gene function, endogenously label genes, or create targeted transgene additions in one or more plant cells. The methods, systems, and compositions disclosed herein may be used to introduce an exogenous gene into a plant cell. The exogenous gene may be a transgene, an artificial gene, an engineered gene, a modified transgene. Alternatively, or in addition, the methods, systems, and compositions disclosed herein may be used to modify an endogenous gene in a plant cell. Modifying an endogenous gene may comprise modifying the coding sequence, modifying the non-coding sequence, altering gene expression, truncating the gene, or creating a gene fusion. A plant comprising a cell with the exogenous gene or the modified endogenous gene may be referred to herein as a modified plant or a genetically modified organism (GMO). The modified plant may express the exogenous gene or the modified endogenous gene to produce an exogenous gene product. For example, the plant may produce an exogenous gene product for bioproduction. In some embodiments, the exogenous gene product may produce an exogenous reaction product. The modified plant may have a desired trait encoded by the exogenous gene or the modified endogenous gene. For example, the modified plant may be drought-resistant, fast-growing, herbicide tolerant, virus-resistant, pest-resistant, or pesticide-resistant. In another example, the modified plant may produce a plant-based product (e.g., a fruit, a vegetable, a grain, a bean, or a seed) with a desired trait. For example, the plant-based product produced by the modified plant may have improved taste, improved shelf life, or improved nutritional value.

The plant can be a monocotyledonous plant. The plant can be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant can belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant can include algae.

In some embodiments, the target nucleic acid sequence comprises a nucleic acid sequence of a virus, a bacterium, or other pathogen responsible for a disease in a plant (e.g., a crop). Methods and compositions of the disclosure can be used to treat or detect a disease in a plant. For example, the methods of the disclosure can be used to target a viral nucleic acid sequence in a plant. A programmable nuclease of the disclosure (e.g., Cas14) can cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant can be an RNA virus. A virus infecting the plant can be a DNA virus. Non-limiting examples of viruses that can be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

Methods of genetically modifying a plant cell may comprise contacting a plant cell comprising a target nucleic acid to a programmable nuclease or a polynucleotide encoding a programmable nuclease, contacting the plant cell to an RNA component or a polynucleotide encoding the RNA component, and modifying the target nucleic acid. Alternatively, or in addition, methods of editing a plant cell may comprise contacting a target nucleic acid to a programmable nuclease and an RNA component, modifying the target nucleic acid, and contacting the modified target nucleic acid to a plant cell. The target nucleic acid may comprise a genome, a chromosome, a plasmid, a gene, a promoter, an untranslated region, an open reading frame, an intron, an exon, or an operator, or the target nucleic acid may comprise a segment of a genome, a chromosome, a plasmid, a gene, a promoter, an untranslated region, an open reading frame, an intron, an exon, or an operator. The programmable nuclease may be a Cas12 programmable nuclease (e.g., a CasY protein), as described herein. The RNA component may be a discrete egRNA system, or the RNA component may be a composite egRNA. The RNA component may comprise a crRNA and an intermediary RNA. Modifying the target nucleic acid may comprise contacting the target nucleic acid with a complex comprising a programmable nuclease, a crRNA that hybridizes to a region of the target nucleic acid, and an intermediary RNA; activating target cleavage activity of the programmable nuclease; and introducing one or more double stranded breaks into the target nucleic acid. In some embodiments, modifying the target nucleic acid may comprise removing a segment of the target nucleic acid between a first double stranded break and a second double stranded break, thereby deleting the segment of the target nucleic acid. In some embodiments, modifying the target nucleic acid may comprise removing a segment of the target nucleic acid between a first double stranded break and a second double stranded break and inserting a donor nucleic acid (e.g., an exogenous gene or a modified endogenous gene) between the first double stranded break and the second double stranded break, thereby replacing the segment of the target nucleic acid with the donor nucleic acid. In some embodiments, modifying the target nucleic acid may comprise inserting a donor nucleic acid (e.g., an exogenous gene or a modified endogenous gene) at a double stranded break, thereby inserting the donor nucleic acid into the target nucleic acid.

In some embodiments, a modified plant cell may have one or more of an altered gene expression, an altered gene product, or an altered phenotype relative to an unmodified plant cell. Editing a plant cell may comprise modifying a chromosome of a plant cell genome. In some embodiments, editing a plant cell may comprise modifying a plasmid of a plant cell. In some embodiments, editing a plant cell may comprise modifying an organelle genome (e.g., a chloroplast genome) of a cell. In some embodiments, the chromosome, plasmid, or organelle genome is modified in the plant cell, thereby producing a modified plant cell. Alternatively, or in addition, the chromosome, plasmid, or organelle genome is modified in vitro and the modified chromosome, plasmid, or organelle genome is introduced into the plant cell, thereby producing a modified plant cell. A plant comprising a modified plant cell may be a modified plant or a genetically modified organism.

As described herein, methods of modifying a target nucleic acid in a plant cell may be used to produce an exogenous gene product or an exogenous reaction product. In some embodiments, the exogenous gene product or the exogenous reaction product may be used for bioproduction. For example, an exogenous gene produced in a modified plant cell may catalyze the synthesis of a vitamin. Alternatively, or in addition, the methods described herein may be used to produce a genetically modified plant having a desired characteristic as compared to an unmodified plant. For example, a genetically modified plant may comprise an exogenous gene or a modified endogenous gene conferring drought-resistance, increased growth rate, herbicide tolerance, virus-resistance, pest-resistance, pesticide-resistance, improved taste, improved shelf life, or improved nutritional value.

b. Detection of Nucleic Acids

The programmable nucleases disclosed herein may exhibit trans cleavage activity upon activation. The trans cleavage activity of the programmable nuclease can be activated when the crRNA is complexed with the target nucleic acid (e.g., viral or bacterial DNA). The trans cleavage activity of the programmable nuclease can be activated when the crRNA and the intermediary RNA are complexed with the target nucleic acid. The target nucleic acid can be a DNA or reverse transcribed RNA, or an amplicon thereof. Preferably, the target nucleic acid is double stranded DNA. Thus, a CasY protein of the present disclosure can be activated by a target DNA to initiate trans cleavage activity of the CasY protein that cleaves a DNA detector nucleic acid. For example, CasY proteins disclosed herein are activated by the binding of the crRNA to a target DNA that was reverse transcribed from an RNA to cleave nucleic acids of a detector nucleic acid in a sequence-independent manner. For example, CasY proteins disclosed herein are activated by the binding of the crRNA to a target DNA that was amplified from a DNA to trans-collaterally cleave detector nucleic acid molecules. The detector nucleic acids can be DNA detector nucleic acids (e.g., single stranded DNA coupled to detectable labels). In some embodiments, the CasY protein recognizes and detects double stranded DNA (dsDNA) and, further, trans cleaves single stranded DNA (ssDNA) detector nucleic acids. Multiple CasY isolates can recognize, be activated by, and detect target DNA as described herein, including dsDNA. Therefore, a programmable nuclease can be used to detect target DNA by assaying for cleaved DNA detector nucleic acids.

The cis cleavage activity of the programmable nuclease can be activated when the crRNA is complexed with the target nucleic acid (e.g., viral or bacterial DNA). The cis cleavage activity of the programmable nuclease can be activated when the crRNA and the intermediary RNA are complexed with the target nucleic acid. The target nucleic acid can be a DNA or reverse transcribed RNA, or an amplicon thereof. Preferably, the target nucleic acid (e.g., viral or bacterial DNA) is double stranded DNA. Thus, a CasY protein of the present disclosure can be activated by a target DNA to initiate cis cleavage activity of the CasY protein that cleaves the target DNA. For example, CasY proteins disclosed herein are activated by the binding of the crRNA to a target DNA that was amplified from a DNA to cleave the target DNA. In some embodiments, the sequence of the target DNA may be modified following cleavage of the target DNA. For example, an insertion sequence may be inserted at the site of cleavage of the target DNA. An insertion sequence may be a DNA sequence (e.g., a ssDNA sequence or a dsDNA sequence) or an RNA sequence. In another example, a segment of the target nucleic acid next to the site of cleavage may be removed from the target nucleic acid (e.g., viral or bacterial DNA). In a further example, a segment of the target nucleic acid next to the site of cleavage may be replaced by an insertion sequence.

In some embodiments, the programmable nuclease may be present in the cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the programmable nuclease may be present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the programmable nuclease may be present in the cleavage reaction at a concentration of from 20 nM to 50 μM, from 50 nM to 20 μM, or from 200 nM to 5 μM.

A programmable nuclease can be used to detect or modify DNA at multiple pH values. A programmable nuclease can be used to detect DNA at multiple pH values. A CasY protein that detects a target DNA can exhibit consistent cleavage across a wide range of pH conditions, such as from a pH of about 8.5 to a pH of about 9.0. In some embodiments, CasY DNA detection may exhibit high cleavage activity at pH values from 6 to 6.5, from 6.1 to 6.6, from 6.2 to 6.7, from 6.3 to 6.8, from 6.4 to 6.9, from 6.5 to 7, from 6.6 to 7.1, from 6.7 to 7.2, from 6.8 to 7.3, from 6.9 to 7.4, from 7 to 7.5, from 7.1 to 7.6, from 7.2 to 7.7, from 7.3 to 7.8, from 7.4 to 7.9, from 7.5 to 8, from 7.6 to 8.1, from 7.7 to 8.2, from 7.8 to 8.3, from 7.9 to 8.4, from 8 to 8.5, from 8.1 to 8.6, from 8.2 to 8.7, from 8.3 to 8.8, from 8.4 to 8.9, from 8.5 to 9, from 8.6 to 9.1, from 8.7 to 9.2, from 8.8 to 9.3, from 8.9 to 9.4, from 9 to 9.5, from 7 to 9, from 7.5 to 9, or from 8 to 9. For example, a programmable nuclease may exhibit high cleavage at a pH of about 8.8.

Target DNA (e.g., viral or bacterial DNA) detected by a programmable nuclease complexed with a crRNA as disclosed herein can be directly obtained from organisms, or can be indirectly generated by nucleic acid amplification methods, such as PCR and LAMP of DNA or reverse transcription of RNA. Key steps for the sensitive detection of direct DNA by a programmable nuclease, such as a CasY protein, can include: (1) production or isolation of DNA to concentrations above about 0.1 nM per reaction for in vitro diagnostics, (2) selection of a target DNA with the appropriate sequence features to enable DNA detection as these some of these features are distinct from those required for target RNA detection, and (3) buffer composition that enhances DNA detection. The detection of DNA by a programmable nuclease can be connected to a variety of readouts including fluorescence, lateral flow, electrochemistry, or any other readouts described herein. Methods for the generation of dsDNA for a DNA-activated programmable RNA nuclease-based detection or diagnostics can include (1) PCR, (2) isothermal amplification, such as RPA, LAMP, SDA, etc. (3) NEAR, and (4) conversion of RNA targets into dsDNA by a reverse transcriptase followed by RNase H digestion and PCR. Thus, a programmable nuclease detection of target DNA is compatible with the various systems, kits, compositions, reagents, and methods disclosed herein. CasY DNA detection can be employed in a DETECTR assay disclosed herein to provide CRISPR diagnostics leveraging Type V systems (e.g., CasY) for the detection of a target DNA (e.g., viral or bacterial DNA).

Some programmable nucleases can exhibit a high turnover rate. Turnover rate quantifies how many molecules of a detector nucleic acid each programmable nuclease is cleaving per minute. Programmable nucleases with a higher turnover rate are more efficient and transcollateral cleavage in the DETECTR assay methods disclosed herein.

Turnover rate is quantified as the max transcleaving velocity (max slope in a plot of signal versus time in a DETECTR assay) divided by the amount of programmable nuclease complexed with the crRNA present in the DETECTR assay, wherein the programmable nuclease is at saturation with respect to its active site for transcollateral cleavage of detector nucleic acids.

Turnover rate can be quantified with the following equation:

$$Turnover rate = \frac{\begin{matrix}maximum transcleaving velocity ( \frac{AU}{min} ) / \\ signal normalization factor ( \frac{AU}{nM} )\end{matrix}}{\begin{matrix}concentration of programmanble nuclease complexed \\ with guide nucleic acid ( nM )\end{matrix}}$$

Signal normalization factor is based on a standard curve and is the amount of signal produced from a known quantity of detector nucleic acid (substrate of transcollateral cleavage). The turnover rate is, thus, expressed as cleaved detector nucleic acid molecules per minute divided by the concentration of the programmable nuclease complexed with an engineered guide RNA system (can also be referred to as “nucleoprotein” or “ribonucleoprotein”). Therefore, a programmable nuclease with a high turnover rate exhibits superior and highly efficient transcollateral cleavage of detector nucleic acids in the DETECTR assay methods disclosed herein. For example, a programmable nuclease that recognizes a PAM of TR, wherein R is A or G, complexed with an egRNA system comprises a turnover rate of at least about 0.01 cleaved detector molecules per minute per programmable nuclease. The programmable nuclease may be a Type V programmable nuclease. The programmable nuclease may be a Cas12 programmable nuclease.

In some embodiments, programmable nucleases with a high turnover rate have a turnover rate of at least about 0.01 cleaved detector molecules per minute per programmable nuclease. In some embodiments, programmable nucleases with a high turnover rate have a turnover rate of at least about 0.05 cleaved detector molecules per minute per programmable nuclease. In some embodiments, programmable nucleases with a high turnover rate have a turnover rate of at least about 0.06 cleaved detector molecules per minute per programmable nuclease. In some embodiments, programmable nucleases with a high turnover rate have a turnover rate of at least about 0.07 cleaved detector molecules per minute per programmable nuclease. In some embodiments, programmable nucleases with a high turnover rate have a turnover rate of at least about 0.08 cleaved detector molecules per minute per programmable nuclease. In some embodiments, programmable nucleases with a high turnover rate have a turnover rate of at least about 0.09 cleaved detector molecules per minute per programmable nuclease. In some embodiments, programmable nucleases with a high turnover rate have a turnover rate of at least about 0.1 cleaved detector molecules per minute per programmable nuclease. In some embodiments, programmable nucleases with a high turnover rate have a turnover rate of at least about 0.11 cleaved detector molecules per minute per programmable nuclease. In some embodiments, programmable nucleases with a high turnover rate have a turnover rate of at least about 0.12 cleaved detector molecules per minute per programmable nuclease. In some embodiments, programmable nucleases with a high turnover rate have a turnover rate of at least about 0.13 cleaved detector molecules per minute per programmable nuclease. In some embodiments, programmable nucleases with a high turnover rate have a turnover rate of at least about 0.14 cleaved detector molecules per minute per programmable nuclease. In some embodiments, programmable nucleases with a high turnover rate have a turnover rate of at least about 0.15 cleaved detector molecules per minute per programmable nuclease. In some embodiments, programmable nucleases with a high turnover rate have a turnover rate of at least about 0.16 cleaved detector molecules per minute per programmable nuclease. In some embodiments, programmable nucleases with a high turnover rate have a turnover rate of at least about 0.17 cleaved detector molecules per minute per programmable nuclease. In some embodiments, programmable nucleases with a high turnover rate have a turnover rate of at least about 0.18 cleaved detector molecules per minute per programmable nuclease. In some embodiments, programmable nucleases with a high turnover rate have a turnover rate of at least about 0.19 cleaved detector molecules per minute per programmable nuclease. In some embodiments, programmable nucleases with a high turnover rate have a turnover rate of at least about 0.20 cleaved detector molecules per minute per programmable nuclease. In some embodiments, programmable nucleases with a high turnover rate have a turnover rate of at least about 0.22 cleaved detector molecules per minute per programmable nuclease. In some embodiments, programmable nucleases with a high turnover rate have a turnover rate of at least about 0.24 cleaved detector molecules per minute per programmable nuclease. In some embodiments, programmable nucleases with a high turnover rate have a turnover rate of at least about 0.26 cleaved detector molecules per minute per programmable nuclease. In some embodiments, programmable nucleases with a high turnover rate have a turnover rate of at least about 0.28 cleaved detector molecules per minute per programmable nuclease. In some embodiments, programmable nucleases with a high turnover rate have a turnover rate of at least about 0.3 cleaved detector molecules per minute per programmable nuclease. In some embodiments, programmable nucleases with a high turnover rate have a turnover rate of at least about 0.4 cleaved detector molecules per minute per programmable nuclease. In some embodiments, programmable nucleases with a high turnover rate have a turnover rate of at least about 0.5 cleaved detector molecules per minute per programmable nuclease. In some embodiments, programmable nucleases with a high turnover rate have a turnover rate of at least about 0.01 to 0.5 cleaved detector molecules per minute per programmable nuclease. In some embodiments, programmable nucleases with a high turnover rate have a turnover rate of at least about 0.01 to 0.2 cleaved detector molecules per minute per programmable nuclease. In some embodiments, programmable nucleases with a high turnover rate have a turnover rate of at least about 0.01 to 0.05 cleaved detector molecules per minute per programmable nuclease. In some embodiments, programmable nucleases with a high turnover rate have a turnover rate of at least about 0.05 to 0.10 cleaved detector molecules per minute per programmable nuclease. In some embodiments, programmable nucleases with a high turnover rate have a turnover rate of at least about 0.10 to 0.15 cleaved detector molecules per minute per programmable nuclease. In some embodiments, programmable nucleases with a high turnover rate have a turnover rate of at least about 0.15 to 0.20 cleaved detector molecules per minute per programmable nuclease. In some embodiments, programmable nucleases with a high turnover rate have a turnover rate of at least about 0.20 to 0.25 cleaved detector molecules per minute per programmable nuclease. In some embodiments, programmable nucleases with a high turnover rate have a turnover rate of at least about 0.25 to 0.30 cleaved detector molecules per minute per programmable nuclease. In some embodiments, programmable nucleases with a high turnover rate have a turnover rate of at least about 0.30 to 0.35 cleaved detector molecules per minute per programmable nuclease. In some embodiments, programmable nucleases with a high turnover rate have a turnover rate of at least about 0.35 to 0.40 cleaved detector molecules per minute per programmable nuclease. In some embodiments, programmable nucleases with a high turnover rate have a turnover rate of at least about 0.40 to 0.45 cleaved detector molecules per minute per programmable nuclease. In some embodiments, programmable nucleases with a high turnover rate have a turnover rate of at least about 0.45 to 0.50 cleaved detector molecules per minute per programmable nuclease. In some embodiments, programmable nucleases with a high turnover rate have a turnover rate of at least about 0.01 to 1 cleaved detector molecules per minute per programmable nuclease. In some embodiments, programmable nucleases with a high turnover rate have a turnover rate of at least about 0.01 to 0.2 cleaved detector molecules per minute per programmable nuclease. In some embodiments, programmable nucleases with a high turnover rate have a turnover rate of at least about 0.01 to 0.3 cleaved detector molecules per minute per programmable nuclease. In some embodiments, programmable nucleases with a high turnover rate have a turnover rate of at least about 0.01 to 0.4 cleaved detector molecules per minute per programmable nuclease. In some embodiments, programmable nucleases with a high turnover rate have a turnover rate of at least about 0.1 to 0.3 cleaved detector molecules per minute per programmable nuclease. In some embodiments, programmable nucleases with a high turnover rate have a turnover rate of at least about 0.2 to 0.4 cleaved detector molecules per minute per programmable nuclease. In some embodiments, programmable nucleases with a high turnover rate have a turnover rate of at least about 0.3 to 0.5 cleaved detector molecules per minute per programmable nuclease. In some embodiments, programmable nucleases with a high turnover rate have a turnover rate of at least about 0.4 to 0.6 cleaved detector molecules per minute per programmable nuclease. In some embodiments, programmable nucleases with a high turnover rate have a turnover rate of at least about 0.5 to 0.7 cleaved detector molecules per minute per programmable nuclease. In some embodiments, programmable nucleases with a high turnover rate have a turnover rate of at least about 0.6 to 0.8 cleaved detector molecules per minute per programmable nuclease. In some embodiments, programmable nucleases with a high turnover rate have a turnover rate of at least about 0.7 to 0.9 cleaved detector molecules per minute per programmable nuclease. In some embodiments, programmable nucleases with a high turnover rate have a turnover rate of at least about 0.8 to 1.0 cleaved detector molecules per minute per programmable nuclease.

Detector Nucleic Acids. Described herein are detector nucleic acids for detecting the presence or absence of a target nucleic acid (e.g., viral or bacterial DNA) in a sample using systems comprising a programmable nuclease (e.g., a CasY protein). The detector nucleic acid can comprise a single stranded nucleic acid and a detection moiety, wherein the nucleic acid is capable of being cleaved by the activated programmable nuclease, releasing the detection moiety, and, generating a detectable signal. The programmable nucleases disclosed herein, activated upon hybridization of a crRNA to a target nucleic acid, can cleave the detector nucleic acid. Specifically, the programmable nucleases disclosed herein, activated upon hybridization of a crRNA to a target nucleic acid, can cleave the nucleic acid of the detector nucleic acid.

A major advantage of the compositions and methods disclosed herein is the design of excess detector nucleic acids to total nucleic acids in an unamplified or an amplified sample, not including the nucleic acid of the detector nucleic acid. Total nucleic acids can include the target nucleic acids and non-target nucleic acids, not including the nucleic acid of the detector nucleic acid. The non-target nucleic acids can be from the original sample, either lysed or unlysed. The non-target nucleic acids can also be byproducts of amplification. Thus, the non-target nucleic acids can include both non-target nucleic acids from the original sample, lysed or unlysed, and from an amplified sample. The presence of a large amount of non-target nucleic acids, an activated programmable nuclease may be inhibited in its ability to bind and cleave the detector nucleic acid sequences. This is because the activated programmable nucleases collaterally cleaves any nucleic acids. If total nucleic acids are in present in large amounts, they may outcompete detector nucleic acids for the programmable nucleases. The compositions and methods disclosed herein are designed to have an excess of detector nucleic acid to total nucleic acids, such that the detectable signals from DETECTR reactions are particularly superior. In some embodiments, the detector nucleic acid can be present in at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold excess of total nucleic acids.

A second significant advantage of the compositions and methods disclosed herein is the design of an excess volume comprising the egRNA system (e.g., discrete egRNA system or composite egRNA), the programmable nuclease, and the detector nucleic acid, which contacts a smaller volume comprising the sample with the target nucleic acid of interest. The smaller volume comprising the sample can be unlysed sample, lysed sample, or lysed sample which has undergone any combination of reverse transcription, amplification, and in vitro transcription. The presence of various reagents in a crude, non-lysed sample, a lysed sample, or a lysed and amplified sample, such as buffer, magnesium sulfate, salts, the pH, a reducing agent, primers, dNTPs, NTPs, cellular lysates, non-target nucleic acids, primers, or other components, can inhibit the ability of the programmable nuclease to become activated or to find and cleave the nucleic acid of the detector nucleic acid. This may be due to nucleic acids that are not the detector nucleic acid outcompeting the nucleic acid of the detector nucleic acid, for the programmable nuclease. Alternatively, various reagents in the sample may simply inhibit the activity of the programmable nuclease. Thus, the compositions and methods provided herein for contacting an excess volume comprising the egRNA system (e.g., discrete egRNA system or composite egRNA), the programmable nuclease, and the detector nucleic acid to a smaller volume comprising the sample with the target nucleic acid of interest provides for superior detection of the target nucleic acid by ensuring that the programmable nuclease is able to find and cleaves the nucleic acid of the detector nucleic acid. In some embodiments, the volume comprising the egRNA system (e.g., discrete egRNA system or composite egRNA), the programmable nuclease, and the detector nucleic acid (can be referred to as “a second volume”) is 4-fold greater than a volume comprising the sample (can be referred to as “a first volume”). In some embodiments, the volume comprising the egRNA system (e.g., discrete egRNA system or composite egRNA), the programmable nuclease, and the detector nucleic acid (can be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold greater than a volume comprising the sample (can be referred to as “a first volume”). In some embodiments, the volume comprising the sample is at least 0.5 μL, at least 1 μL, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, from 0.5 μL to 5 μL, from 5 μL to 10 μL, from 10 μL to 15 μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 10 μL to 20 μL, from 5 μL to 20 μL, from 1 μL to 40 μL, from 2 μL to 10 μL, or from 1 μL to 10 pt. In some embodiments, the volume comprising the programmable nuclease, the egRNA system (e.g., discrete egRNA system or composite egRNA), and the detector nucleic acid is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, from 10 μL to 15 μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 50 μL to 55 μL, from 55 μL to 60 μL, from 60 μL to 65 μL, from 65 μL to 70 μL, from 70 μL to 75 μL, from 75 μL to 80 μL, from 80 μL to 85 μL, from 85 μL to 90 μL, from 90 μL to 95 μL, from 95 μL to 100 μL, from 100 μL to 150 μL, from 150 μL to 200 μL, from 200 μL to 250 μL, from 250 μL to 300 μL, from 300 μL to 350 μL, from 350 μL to 400 μL, from 400 μL to 450 μL, from 450 μL to 500 μL, from 10 μL to 20 μL, from 10 μL to 30 μL, from 25 μL to 35 μL, from 10 μL to 40 μL, from 20 μL to 50 μL, from 18 μL to 28 μL, or from 17 μL to 22 μL.

The nucleic acid of a detector nucleic acid can be a single-stranded nucleic acid sequence comprising at least one deoxyribonucleotide and at least one ribonucleotide. In some cases, the nucleic acid of a detector nucleic acid is a single-stranded nucleic acid comprising at least one ribonucleotide residue at an internal position that functions as a cleavage site. In some cases, the nucleic acid of a detector nucleic acid comprises at least 2, 3, 4, 5, 6, 7, 8, 9, or 10 ribonucleotide residues at an internal position. In some cases, the nucleic acid of a detector nucleic acid comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the nucleic acid of a detector nucleic acid has only ribonucleotide residues. In some cases, the nucleic acid of a detector nucleic acid has only deoxyribonucleotide residues. In some cases, the nucleic acid comprises nucleotides resistant to cleavage by the programmable nuclease described herein. In some cases, the nucleic acid of a detector nucleic acid comprises synthetic nucleotides. In some cases, the nucleic acid of a detector nucleic acid comprises at least one ribonucleotide residue and at least one non-ribonucleotide residue. In some cases, the nucleic acid of a detector nucleic acid is 5-20, 5-15, 5-10, 7-20, 7-15, or 7-10 nucleotides in length. In some cases, the nucleic acid of a detector nucleic acid is from 3 to 20, from 4 to 10, from 5 to 10, or from 5 to 8 nucleotides in length. In some cases, the nucleic acid of a detector nucleic acid comprises at least one uracil ribonucleotide. In some cases, the nucleic acid of a detector nucleic acid comprises at least two uracil ribonucleotides. Sometimes the nucleic acid of a detector nucleic acid has only uracil ribonucleotides. In some cases, the nucleic acid of a detector nucleic acid comprises at least one adenine ribonucleotide. In some cases, the nucleic acid of a detector nucleic acid comprises at least two adenine ribonucleotides. In some cases, the nucleic acid of a detector nucleic acid has only adenine ribonucleotides. In some cases, the nucleic acid of a detector nucleic acid comprises at least one cytosine ribonucleotide. In some cases, the nucleic acid of a detector nucleic acid comprises at least two cytosine ribonucleotides. In some cases, the nucleic acid of a detector nucleic acid comprises at least one guanine ribonucleotide. In some cases, the nucleic acid of a detector nucleic acid comprises at least two guanine ribonucleotides. A nucleic acid of a detector nucleic acid can comprise only unmodified ribonucleotides, only unmodified deoxyribonucleotides, or a combination thereof. In some cases, the nucleic acid of a detector nucleic acid is from 5 to 12 nucleotides in length. In some cases, the nucleic acid of a detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the nucleic acid of a detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising CasY protein, a nucleic acid of a detector nucleic acid can be 5, 8, or 10 nucleotides in length. For cleavage by a programmable nuclease comprising Cas12, a nucleic acid of a detector nucleic acid can be 10 nucleotides in length.

The single stranded nucleic acid of a detector nucleic acid comprises a detection moiety capable of generating a first detectable signal. Sometimes the detector nucleic acid comprises a protein capable of generating a signal. A signal can be a calorimetric, potentiometric, amperometric, optical (e.g., fluorescent, colorimetric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the nucleic acid of a detector nucleic acid. Sometimes the detection moiety is at the 3′ terminus of the nucleic acid of a detector nucleic acid. In some cases, the detection moiety is at the 5′ terminus of the nucleic acid of a detector nucleic acid. In some cases, the quenching moiety is at the 3′ terminus of the nucleic acid of a detector nucleic acid. In some cases, the single-stranded nucleic acid of a detector nucleic acid is at least one population of the single-stranded nucleic acid capable of generating a first detectable signal. In some cases, the single-stranded nucleic acid of a detector nucleic acid is a population of the single stranded nucleic acid capable of generating a first detectable signal. Optionally, there is more than one population of single-stranded nucleic acid of a detector nucleic acid. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded nucleic acids of a detector nucleic acid capable of generating a detectable signal. In some cases, there are from 2 to 50, from 3 to 40, from 4 to 30, from 5 to 20, or from 6 to 10 different populations of single-stranded nucleic acids of a detector nucleic acid capable of generating a detectable signal.

TABLE 2
Exemplary Single Stranded Nucleic Acids in a Detector Nucleic Acid

| 5′ Detection Moiety* | Sequence (SEQ ID NO) | 3′ Quencher* |
|---|---|---|
| /56-FAM/ | rUrUrUrUrU (SEQ ID NO: 11) | /3IABkFQ/ |
| /5IRD700/ | rUrUrUrUrU (SEQ ID NO: 11) | /3IRQC1N/ |
| /5TYE665/ | rUrUrUrUrU (SEQ ID NO: 11) | /3IAbRQSp/ |
| /5Alex594N/ | rUrUrUrUrU (SEQ ID NO: 11) | /3IAbRQSp/ |
| /5ATTO633N/ | rUrUrUrUrU (SEQ ID NO: 11) | /3IAbRQSp/ |
| /56-FAM/ | rUrUrUrUrUrUrUrU(SEQ ID NO: 12) | /3IABkFQ/ |
| /5IRD700/ | rUrUrUrUrUrUrUrU(SEQ ID NO: 12) | /3IRQC1N/ |
| /5TYE665/ | rUrUrUrUrUrUrUrU(SEQ ID NO: 12) | /3IAbRQSp/ |
| /5Alex594N/ | rUrUrUrUrUrUrUrU(SEQ ID NO: 12) | /3IAbRQSp/ |
| /5ATTO633N/ | rUrUrUrUrUrUrUrU(SEQ ID NO: 12) | /3IAbRQSp/ |
| /56-FAM/ | rUrUrUrUrUrUrUrUrUrU(SEQ ID NO: 13) | /3IABkFQ/ |
| /5IRD700/ | rUrUrUrUrUrUrUrUrUrU(SEQ ID NO: 13) | /3IRQC1N/ |
| /5TYE665/ | rUrUrUrUrUrUrUrUrUrU(SEQ ID NO: 13) | /3IAbRQSp/ |
| /5Alex594N/ | rUrUrUrUrUrUrUrUrUrU(SEQ ID NO: 13) | /3IAbRQSp/ |
| /5ATTO633N/ | rUrUrUrUrUrUrUrUrUrU(SEQ ID NO: 13) | /3IAbRQSp/ |
| /56-FAM/ | TTTTrUrUTTTT(SEQ ID NO: 14) | /3IABkFQ/ |
| /5IRD700/ | TTTTrUrUTTTT(SEQ ID NO: 14) | /3IRQC1N/ |
| /5TYE665/ | TTTTrUrUTTTT(SEQ ID NO: 14) | /3IAbRQSp/ |
| /5Alex594N/ | TTTTrUrUTTTT(SEQ ID NO: 14) | /3IAbRQSp/ |
| /5ATTO633N/ | TTTTrUrUTTTT(SEQ ID NO: 14) | /3IAbRQSp/ |
| /56-FAM/ | TTrUrUTT(SEQ ID NO: 15) | /3IABkFQ/ |
| /5IRD700/ | TTrUrUTT(SEQ ID NO: 15) | /3IRQC1N/ |
| /5TYE665/ | TTrUrUTT(SEQ ID NO: 15) | /3IAbRQSp/ |
| /5Alex594N/ | TTrUrUTT(SEQ ID NO: 15) | /3IAbRQSp/ |
| /5ATTO633N/ | TTrUrUTT(SEQ ID NO: 15) | /3IAbRQSp/ |
| /56-FAM/ | TArArUGC(SEQ ID NO: 16) | /3IABkFQ/ |
| /5IRD700/ | TArArUGC(SEQ ID NO: 16) | /3IRQC1N/ |
| /5TYE665/ | TArArUGC(SEQ ID NO: 16) | /3IAbRQSp/ |
| /5Alex594N/ | TArArUGC(SEQ ID NO: 16) | /3IAbRQSp/ |
| /5ATTO633N/ | TArArUGC(SEQ ID NO: 16) | /3IAbRQSp/ |
| /56-FAM/ | TArUrGGC(SEQ ID NO: 17) | /3IABKFQ/ |
| /5IRD700/ | TArUrGGC(SEQ ID NO: 17) | /3IRQC1N/ |
| /5TYE665/ | TArUrGGC(SEQ ID NO: 17) | /3IAbRQSp/ |
| /5Alex594N/ | TArUrGGC(SEQ ID NO: 17) | /3IAbRQSp/ |
| /5ATTO633N/ | TArUrGGC(SEQ ID NO: 17) | /3IAbRQSp/ |
| /56-FAM/ | rUrUrUrUrU(SEQ ID NO: 18) | /3IABkFQ/ |
| /5IRD700/ | rUrUrUrUrU(SEQ ID NO: 18) | /3IRQC1N/ |
| /5TYE665/ | rUrUrUrUrU(SEQ ID NO: 18) | /3IAbRQSp/ |
| /5Alex594N/ | rUrUrUrUrU(SEQ ID NO: 18) | /3IAbRQSp/ |
| /5ATTO633N/ | rUrUrUrUrU(SEQ ID NO: 18) | /3IAbRQSp/ |
| /56-FAM/ | TTATTATT (SEQ ID NO: 19) | /3IABkFQ/ |
| /56-FAM/ | TTATTATT (SEQ ID NO: 19) | /3IABkFQ/ |
| /5IRD700/ | TTATTATT (SEQ ID NO: 19) | /3IRQC1N/ |
| /5TYE665/ | TTATTATT (SEQ ID NO: 19) | /3IAbRQSp/ |
| /5Alex594N/ | TTATTATT (SEQ ID NO: 19) | /3IAbRQSp/ |
| /5ATTO633N/ | TTATTATT (SEQ ID NO: 19) | /3IAbRQSp/ |
| /56-FAM/ | TTTTTT (SEQ ID NO: 20) | /3IABkFQ/ |
| /56-FAM/ | TTTTTTTT (SEQ ID NO: 21) | /3IABkFQ/ |
| /56-FAM/ | TTTTTTTTTT (SEQ ID NO: 22) | /3IABkFQ/ |
| /56-FAM/ | TTTTTTTTTTTT (SEQ ID NO: 23) | /3IABkFQ/ |
| /56-FAM/ | TTTTTTTTTTTTTT (SEQ ID NO: 24) | /3IABkFQ/ |
| /56-FAM/ | AAAAAA (SEQ ID NO: 25) | /3IABkFQ/ |
| /56-FAM/ | CCCCCC (SEQ ID NO: 26) | /3IABkFQ/ |
| /56-FAM/ | GGGGGG (SEQ ID NO: 27) | /3IABkFQ/ |
| /56-FAM/ | TTATTATT (SEQ ID NO: 19) | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQC1N/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the detection moiety emits fluorescence in the range from 450 nm to 750 nm, from 500 nm to 650 nm, or from 550 to 650 nm. A detection moiety can be a fluorophore that emits a detectable fluorescence signal in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 11 with a fluorophore that emits a fluorescence around 520 nm is used for testing in non-urine samples, and SEQ ID NO: 18 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range from 450 nm to 750 nm, from 500 nm to 650 nm, or from 550 to 650 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nuclease has occurred and that the sample contains the target nucleic acid (e.g., viral or bacterial DNA). In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively. or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A nucleic acid of a detector nucleic acid, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a detector nucleic acid. Sometimes, a calorimetric signal is heat absorbed after cleavage of the nucleic acids of a detector nucleic acid. A potentiometric signal, for example, is electrical potential produced after cleavage of the nucleic acids of a detector nucleic acid. An amperometric signal can be movement of electrons produced after the cleavage of nucleic acid of a detector nucleic acid. Often, the signal is an optical signal, such as a colorimetric signal or a fluorescence signal. An optical signal is, for example, a light output produced after the cleavage of the nucleic acids of a detector nucleic acid. Sometimes, an optical signal is a change in light absorbance between before and after the cleavage of nucleic acids of a detector nucleic acid. Often, a piezo-electric signal is a change in mass between before and after the cleavage of the nucleic acid of a detector nucleic acid.

Often, the protein-nucleic acid is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose. A DNS reagent produces a colorimetric change when invertase converts sucrose to glucose. In some cases, it is preferred that the nucleic acid (e.g., DNA) and invertase are conjugated using a heterobifunctional linker via sulfo-SMCC chemistry. Sometimes the protein-nucleic acid is a substrate-nucleic acid. Often the substrate is a substrate that produces a reaction with an enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorimetric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of egRNA system (e.g., discrete egRNA system or composite egRNA) and more than one type of nucleic acid of a detector nucleic acid. In some cases, the detectable signal is generated directly by the cleavage event. Alternatively. or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases the threshold of detection is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid is detected in a sample is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, the target nucleic acid is present in the cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, the methods, compositions, reagents, enzymes, and kits described herein may be used to detect a target single-stranded nucleic acid in a sample where the sample is contacted with the reagents for a predetermined length of time sufficient for the trans cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, the devices, systems, fluidic devices, kits, and methods described herein can detect a target nucleic acid in a sample in from 5 minutes to 10 hours, from 10 minutes to 8 hours, from 15 minutes to 6 hours, from 20 minutes to 5 hours, from 30 minutes to 2 hours, or from 45 minutes to 1 hour.

When a crRNA binds to a target nucleic acid, the programmable nuclease's trans cleavage activity can be initiated, and nucleic acids of a detector nucleic acid can be cleaved, resulting in the detection of fluorescence. The crRNA may be a non-naturally occurring crRNA. A non-naturally occurring crRNA may comprise an engineered sequence having a repeat and a spacer that hybridizes to a target nucleic acid sequence of interest. A non-naturally occurring crRNA may be recombinantly expressed or chemically synthesised. Nucleic acid detector nucleic acids can comprise a detection moiety, wherein the nucleic acid detector nucleic acid can be cleaved by the activated programmable nuclease, thereby generating a signal. Some methods as described herein can a method of assaying for a target nucleic acid in a sample comprises contacting the sample to a complex comprising a crRNA comprising a segment that is reverse complementary to a segment of the target nucleic acid and a programmable nuclease that exhibits sequence independent cleavage upon forming a complex comprising the segment of the crRNA binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The cleaving of the nucleic acid of a detector nucleic acid using the programmable nuclease may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a crRNA targeting a target nucleic acid segment, a programmable nuclease capable of being activated when complexed with the crRNA and the target nucleic acid segment, a single stranded nucleic acid of a detector nucleic acid comprising a detection moiety, wherein the nucleic acid of a detector nucleic acid is capable of being cleaved by the activated programmable nuclease, thereby generating a first detectable signal, cleaving the single stranded nucleic acid of a detector nucleic acid using the programmable nuclease that cleaves as measured by a change in color, and measuring the first detectable signal on the support medium. The cleaving of the single stranded nucleic acid of a detector nucleic acid using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a crRNA targeting a target nucleic acid segment, a programmable nuclease capable of being activated when complexed with the crRNA and the target nucleic acid segment, and a single stranded nucleic acid of a detector nucleic acid comprising a detection moiety, wherein the nucleic acid of a detector nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample. In some embodiments, the first detectable signal can be detectable within from 1 to 120, from 5 to 100, from 10 to 90, from 15 to 80, from 20 to 60, or from 30 to 45 minutes of contacting the sample.

In some cases, the methods, reagents, enzymes, and kits described herein detect a target single-stranded nucleic acid with a programmable nuclease and a single-stranded nucleic acid of a detector nucleic acid in a sample where the sample is contacted with the reagents for a predetermined length of time sufficient for trans cleavage of the single stranded nucleic acid of a detector nucleic acid. In a preferred embodiment, a CasY protein may be used to detect the presence of a single-stranded DNA target nucleic acid. For example, a programmable nuclease is CasY protein that detects a target nucleic acid and a single stranded nucleic acid of a detector nucleic acid with a green detectable moiety that is detected upon cleavage. As another example, a programmable nuclease is CasY protein that detects a target nucleic acid and a single-stranded nucleic acid of a detector nucleic acid with a red detectable moiety that is detected upon cleavage.

### Target Nucleic Acids

A number of different target nucleic acids can be detected with the compositions and methods disclosed herein. For example, the target nucleic acid may be bacterial or viral DNA. Viral DNA may be from from papovavirus, human papillomavirus (HPV), hepadnavirus, Hepatitis B Virus (HBV), herpesvirus, varicella zoster virus (VZV), epstein-barr virus (EBV), kaposi's sarcoma-associated herpesvirus, adenovirus, poxvirus, or parvovirus, an influenza virus, a respiratory syncytial virus, or a coronavirus. An influenza virus may be Influenza A or Influenza B. A coronavirus may include SARS-CoV2 or any other strain of coronavirus. In some embodiments, the target nucleic acid sequence comprises a nucleic acid sequence of a virus or a bacterium or other agents responsible for a disease in the sample. In some embodiments, the target nucleic acid comprises DNA. The target nucleic acid, in some cases, is a portion of a nucleic acid from a sexually transmitted infection or a contagious disease, in the sample. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus; immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment. In some cases, the mutation that confers resistance to a treatment is a deletion

### EXAMPLES

The following examples are illustrative and non-limiting to the scope of the devices, systems, compositions, kits, and methods described herein.

### Example 1

### Engineering crRNAs for Use with a Programmable Nuclease

This example describes engineering crRNAs for use with a programmable nuclease of the present disclosure. The length and sequence of the repeat and the spacer of crRNAs were varied to assess for use with a programmable nuclease comprising a CasY protein.

### Engineering Repeats

The repeats of native CasY CRISPR RNAs are typically 25 nucleotides in length and are positioned 5′ to the spacer sequence, as shown in FIG. 1A. The crRNA has a spacer with a sequence that is reverse complementary to a sequence of the target nucleic acid and a repeat having an “AAGGC” sequence upstream of the spacer. Finally, an intermediary RNA is shown having a sequence reverse complementary to the “AAGGC” sequence in the repeat of the crRNA. The intermediary RNA binds to a programmable nuclease (e.g., a CasY protein, also referred to as “Cas12d protein”) (FIG. 1A). The composite egRNA has a crRNA linked to an intermediary RNA (FIG. 1B). The guide system depicted in FIG. 1B may be internal to a larger engineered guide system, with the residues depicted in FIG. 1B essential to full activity of the guide. To determine the optimal and minimal lengths of the repeat, crRNAs with repeats of varying length were screened for the ability to activate CasY trans cleavage activity. crRNAs with varying repeats were screened using a DETECTR trans cleavage activity. Briefly, the crRNAs were combined with an intermediary RNA, a CasY3 programmable nuclease, an intermediary RNA, a target nucleic acid, and a detector nucleic acid. Upon activation, the programmable nuclease cleaves the detector nucleic acid, producing a detectable signal. Surprisingly, the results of this assay indicated that that the crRNA with a repeat that was 25 nucleotides in length did not elicit the most trans cleavage activity. Unexpectedly, the results showed that a crRNA with a short repeat, from 5 to 10 nucleotides in length, elicited greater trans cleavage activity than the native 25 nucleotide repeat when complexed with a programmable nuclease disclosed herein.

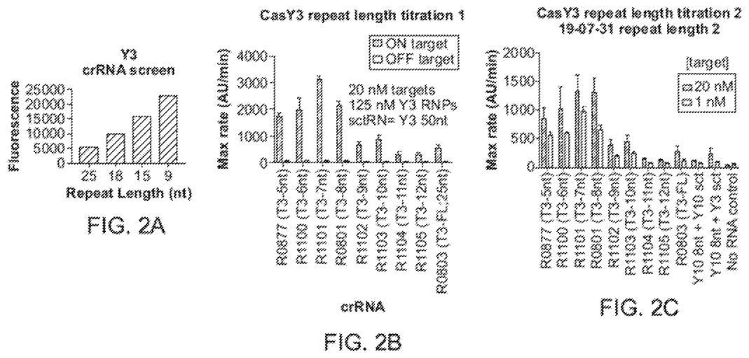

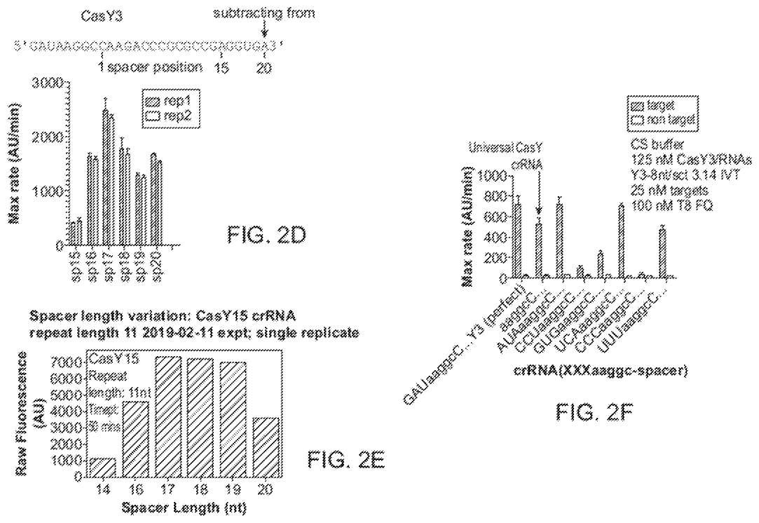

In one DETECTR reaction, a CasY3 programmable nuclease was incubated for 2 hours with crRNAs with varying repeat lengths, including 25 nucleotides, 18 nucleotides, 15 nucleotides, and 9 nucleotides. FIG. 2A shows a graph of fluorescence from 2-hour DETECTR reactions in which the length of the repeat of the crRNA was varied. The highest fluorescence signal was observed in the DETECTR reaction in which the crRNA repeat was 9 nucleotides in length. The DETECTR reaction contained 125 nM crRNA, 125 nM intermediary RNA, 100 nM reporter, 100 nM CasY3 programmable nuclease (SEQ ID NO: 3), and 20 nM target nucleic acid (GFP-T3, SEQ ID NO: 42). The sequences of the crRNAs and intermediary RNA used in the DETECTR reaction are provided in TABLE 3.

TABLE 3
Reagents used in the DETECTR Reaction

| SEQ | | | |
|---|---|---|---|
| ID NO | Name | Sequence | Concentration |
| SEQ ID | 25 nt repeat | CUCCGAAUUAUCGGG | 125 nM |
| NO: 28 | crRNA | AGGAUAAGGCCAAGA | |
| | (R803) | CCCGCGCCGAGGU | |
| SEQ ID | 18 nt repeat | UUAUCGGGAGGAUAA | 125 nM |
| NO: 29 | crRNA | GGCCAAGACCCGCGC | |
| | | CGAGGU | |
| SEQ ID | 15 nt repeat | GGGAGGAUAAGGCCA | 125 nM |
| NO: 30 | crRNA | AGACCCGCGCCGAGG | |
| | | U | |
| SEQ ID | 9 nt repeat | GGAUAAGGCCAAGAC | 125 nM |
| NO: 31 | crRNA | CCGCGCCGAGGU | |
| | (R1102) | | |
| SEQ ID | Intermediary | CUCCGAAUUAUCGGG | 125 nM |
| NO: 32 | RNA (Y3.4) | AGGAUAAGUAUGGAU | |
| | | AUUUCCACAAUCUUG | |
| | | AAAGAAAGAUUUGUU | |
| | | AGCCUUUAAUCCAUU | |
| | | CUCCUUUCCCUUUAU | |
| | | UUUAUCUGACAACAU | |

In another DETECTR reaction, 125 nM of a CasY3 programmable nuclease (SEQ ID NO: 3) was incubated with crRNAs with varying repeat lengths and an intermediary RNAs in the presence of 20 nM of the target nucleic acid. FIG. 2B shows a graph of results from DETECTR reactions with 20 nM of the target nucleic acid, in which the length of the repeat of the crRNA was varied. The graph shows the max rate (AU/min) for each assay condition. Max rate is the highest cleavage per unit time measured in a 5-minute window of the DETECTR reaction. Typically, transcleavage rates increase early in the reaction as the temperature equilibrates and target binding completes and plateau later in in the reaction until the reporter is consumed. The Plateau typically occurs around the maximum rate. The reagents used in the DETECTR reaction are provided in TABLE 4.

TABLE 4
Reagents used in the DETECTR Reaction

| SEQ ID | | | |
|---|---|---|---|
| NO: | Description | Name | Sequence |
| SEQ ID | Minimized | R1083 (Y3 min | AUGGAUAUUUCCACAAUCUU |
| NO: 33 | Intermediary RNA | 50) | GAAAGAAAGAUUUGUUAGCC |
| | | | UUUAAUCCAU |
| SEQ ID | crRNA 5 nt repeat | R877 (T3-5nt) | AAGGCCAAGACCCGCGCCGAG |
| NO: 34 | targeting GFP target 3 | | GU |
| SEQ ID | crRNA 6 nt repeat | R1100 (T3-6nt) | UAAGGCCAAGACCCGCGCCGA |
| NO: 35 | targeting GFP target 3 | | GGU |
| SEQ ID | crRNA 7 nt repeat | R1101 (T3-7nt) | AUAAGGCCAAGACCCGCGCCG |
| NO: 36 | targeting GFP target 3 | | AGGU |
| SEQ ID | crRNA 8 nt repeat | R801 (T3-8nt) | GAUAAGGCCAAGACCCGCGCC |
| NO: 37 | targeting GFP target 3 | | GAGGU |
| SEQ ID | crRNA 9 nt repeat | R1102 (T3-9nt) | GGAUAAGGCCAAGACCCGCGC |
| NO: 31 | targeting GFP target 3 | | CGAGGU |
| SEQ ID | crRNA 10 nt repeat | R1103 (T3- | AGGAUAAGGCCAAGACCCGC |
| NO: 38 | targeting GFP target 3 | 10nt) | GCCGAGGU |
| SEQ ID | crRNA 11 nt repeat | R1104 (T3- | GAGGAUAAGGCCAAGACCCG |
| NO: 39 | targeting GFP target 3 | 11nt) | CGCCGAGGU |
| SEQ ID | crRNA 12 nt repeat | R1105 (T3-12 | GGAGGAUAAGGCCAAGACCC |
| NO: 40 | targeting GFP target 3 | nt) | GCGCCGAGGU |
| SEQ ID | crRNA Full length | R803 (T3-25 nt) | CUCCGAAUUAUCGGGAGGAU |
| NO: 28 | repeat targeting GFP | | AAGGCCAAGACCCGCGCCGAG |
| | T3 | | GU |
| SEQ ID | Functional | Y3.14 | UCGGGAGGAUAAGUAUGGAU |
| NO: 41 | intermediary RNA pre- | | AUUUCCACAAUCUUGAAAGA |
| | minimization | | AAGAUUUGUUAGCCUUUAAU |
| | | | CCAUUCUCCUUUCCCUUUAUU |
| | | | UUAUCUGACAACAU |
| SEQ ID | Target DNA 200 bp | GFP-T3 | CATGAAGCAGCACGACTTCTT |
| NO: 42 | fragment of GFP | | CAAGTCCGCCATGCCCGAAGG |
| | containing target 3 | | CTACGTCCAGGAGCGCACCAT |
| | | | CTTCTTCAAGGACGACGGCAA |
| | | | CTACAAGACCCGCGCCGAGGT |
| | | | GAAGTTCGAGGGCGACACCCT |
| | | | GGTGAACCGCATCGAGCTGAA |
| | | | GGGCATCGACTTCAAGGAGGA |
| | | | CGGCAACATCCTGGGGCACAA |
| | | | GCTGGAGTACA |
| SEQ ID | reporter | T8 | F-TTTTTTTT-Q |
| NO: 21 | | | |

In another DETECTR reaction, a CasY3 programmable nuclease was incubated with crRNAs with varying repeat lengths and an intermediary RNA in the presence of 20 nM of the target nucleic acid or 1 nM of the target nucleic acid. FIG. 2C shows a graph of results from DETECTR reactions with 20 nM or 1 nM of the target nucleic acid, in which the length of the repeat of the crRNA was varied. The graph shows the max rate (AU/min) for each assay condition. The reagents used in the DETECTR reaction are provided in TABLE 4.

As demonstrated in FIG. 2A-FIG. 2C, CasY3 displayed approximately 4-fold to 8-fold higher target-dependent trans cleavage activity when the ribonucleoprotein (RNP) was assembled with a shortened repeat segment crRNA (FIG. 2A-FIG. 2C). The extent of enhancement in the reaction depended on the buffer conditions. The crRNAs with shorted repeats identified in FIG. 2A as eliciting increased trans nuclease activity of CasY3 were further optimized. A preferred repeat length of from 7 to 8 nucleotides was identified (FIG. 2B). The preferred repeat included the 5 conserved nucleotides (AAGGC) reverse complementary to the intermediary RNA, plus two or three additional bases 5′ of the 5 conserved nucleotides (AAGGC) (FIG. 2B). These results suggested that the first two nucleotides upstream of the conserved AAGGC have an impact on activity of the CasY3 protein. Enhanced activity imparted by truncating the repeat of the crRNA was critical to achieving a level of activity with CasY3, and potentially other CasY proteins, suitable for a desired application.

A crRNA having a repeat with only the 5-nucleotide sequence AAGGC was tested to evaluate if it could function as a “universal crRNA” for use with an intermediary RNA containing the reverse complementary GCCTT sequence and any CasY protein (FIG. 1A). As seen in FIG. 2B-FIG. 2C, the crRNA with a repeat with only the AAGGC sequence (“T3-5nt”) was functional and elicited activity of a CasY3 programmable nuclease. The 5-nucleotide repeat elicited CasY3 nuclease activity greater than the activity observed for a crRNA with the native 25-nucleotide repeat crRNA but less than the activity observed with a crRNA having the preferred 7-8 nucleotide repeat (FIG. 2B-FIG. 2C). These results suggested that RNA components may be designed that can be utilized by different CasY proteins in the same setting, for applications in gene modification and/or detections of target nucleic acids (e.g., with a DETECTR assay).

Nucleotides located upstream (5′) of the AAGGC sequence of the repeat of the crRNA, are not reverse complementary to the intermediary RNA. To determine the effect of these nucleotides on CasY activation, crRNAs having different sequences 5′ of the AAGGC sequence of the repeat were screened. Unexpectedly, the results of this assay showed that the sequence identity of the residues 5′ of the AAGGC sequence was crucial for trans cleavage activity. Six different crRNAs having distinct 3-nucleotide sequences positioned upstream of the AAGGC sequence were evaluated in a DETECTR assay. Of the six crRNAs tested, some sequences were fully permissive for CasY3 trans cleavage activity, others were inhibitory, and some nearly fully inhibited the assay (FIG. 2F). In a series of DETECTR assays, 125 nM of a CasY3 programmable nuclease, an intermediary RNA (SEQ ID NO: 32), crRNAs with repeats with varying 3 nucleotide sequences upstream of an AAGGC sequence, 25 nM of the target nucleic acid, and 100 nM of a T8 FQ detector nucleic acid (SEQ ID NO: 21) was tested. crRNA repeats tested included the following 3-nucleotide sequences upstream of the AAGGC sequence: GAU, no 3-nucleotide sequence, AUA, CCU, GUG, UCA, CCC, and UUU. FIG. 2F shows a graph of results from DETECTR reactions in which various repeats either 8 nucleotides in length (AAGGC+3 nucleotides at the 5′ end) or a “universal” AAGGC repeat was tested. The graph shows the max rate (AU/min) for each assay condition. The results demonstrated that only two nucleotides in addition to the AAGGC sequence were critical to improved activity.

Some 8 nucleotide repeat (5′+3 nucleotides-AAGGC:Spacer) crRNAs functioned between orthologs. For example, we found that CasY15+3 sequence is compatible for supporting trans cleavage with CasY3 RNP. Thus, we have generated crRNAs with repeats that are either permissive or restrictive to eliciting trans cleavage activity of CasY proteins, potentially differentiating between orthologs. Some crRNA sequences having a sequence of NNNAAGGC, where N is any nucleotide, were functional between programmable nuclease orthologs, while others were found to be functional or non-functional with CasY3. These results suggested that it is possible to designed ortholog-specific crRNA+3 sequences that are functional with some programmable nuclease orthologs but not with others.

### Engineering Spacers

The effect of spacer length on CasY trans cleavage activity was also investigated in DETECTR reactions. Spacer sequences were directed to a target nucleic acid having a “TA” PAM and coding for GFP. In a series of DETECTR assays, a CasY3 programmable nuclease, an intermediary RNA, a target nucleic acid, a detector nucleic acid, and crRNAs with a constant repeat (GAUAAGGC) but variable spacer lengths were tested. The assay was run in duplicate (rep1 and rep2). Spacers were varied as shown at the top of the graph in FIG. 2D. FIG. 2D shows the results from the DETECTR reactions in which the length of the spacer of the crRNA was varied. The graph shows the max rate (AU/min) for each assay condition. As seen from the results, spacers between 15 and 20 nucleotides and as short as 16 nucleotides supported the reaction. A clear optimum in activity was achieved with a 17-nucleotide spacer (FIG. 2D). Assays were performed using 125 nM of an R1083 crRNA (SEQ ID NO: 33) with 125 nM programmable nuclease, 25 nM GFP-T3 target (SEQ ID NO: 42), and 100 nM reporter.

In another series of DETECTR assays, a CasY15 programmable nuclease (SEQ ID NO: 10), an intermediary RNA, a target nucleic acid, a detector nucleic acid, and crRNAs with a constant repeat but variable spacer lengths were tested. FIG. 2E shows a graph of results from 50-min DETECTR reactions in which the length of the spacer of the crRNA was varied. The graph shows fluorescence from cleavage of the detector nucleic acid in the DETECTR assay for each assay condition. The results demonstrated that for the target sequence tested, the optimal spacer length for the CasY15 programmable nuclease was also 17-19 nucleotides (FIG. 2E). This assay used a Y15 intermediary RNA (SEQ ID NO: 48), an 11-nucleotide repeat (SEQ ID NO: 118, GCGAUGAAGGC), and an annealed oligonucleotide target. The final concentrations of the reagents used in the assay were 100 nM CasY15 programmable nuclease (SEQ ID NO: 10), 125 nM crRNA, 125 nM intermediary RNA, 50 nM Fluor-Quencher reporter, and 2 nM target (activator).

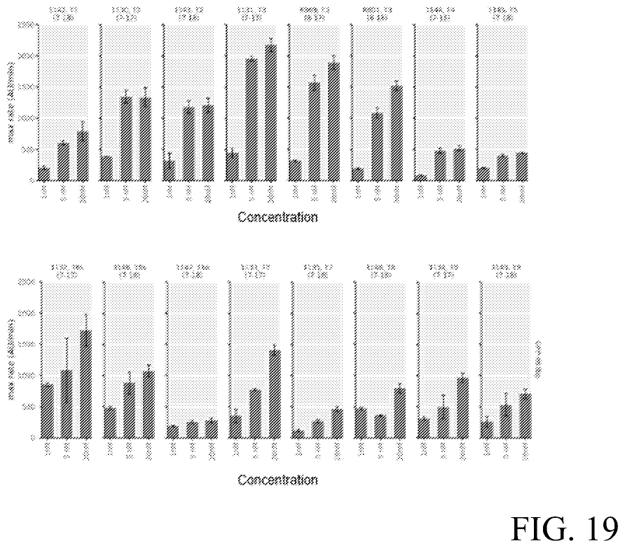

The 17 and 18 nucleotide spacer lengths were tested in another five targets within GFP and the results demonstrated that, in each case, the 17-nucleotide spacer supported higher trans cleavage, as shown in FIG. 19. Different GFP target sites (T1-T9, from left to right and top to bottom in FIG. 19, T3 corresponds to SEQ ID NO: 42) were targeted by as Y3 (SEQ ID NO: 3) and various crRNAs. crRNAs contained either a 7 nucleotide or 8-nucleotide repeat and either a 17 nucleotide or 18 nucleotide spacer. crRNAs are denoted at the top of each plot in FIG. 19 in parentheses as: (repeat length-spacer length). Depending on the target, the 17-nucleotide spacer supported trans cleavage up to nearly 3-fold over the corresponding 18 nucleotide spacer crRNA. Therefore, an in vitro spacer length of 17 nucleotides in conjunction with a CasY3 programmable nuclease was optimal across a range of different target sequence, though it is possible some target sequences will differ.

Together with the optimized repeat length, the optimized spacer helped achieve the highest specific activities possible for CasY proteins in various applications.

### Example 2

### Engineering Intermediary RNAs for Use with a Programmable Nuclease

This example describes engineering intermediary RNAs for use with a programmable nuclease of the present disclosure.

Defining the minimal intermediary RNA structure for CasY activity

Intermediary RNA sequences for various CasY orthologs were initially selected based on the presence of a GCCTT motif in the non-coding DNA surrounding the CRISPR locus.

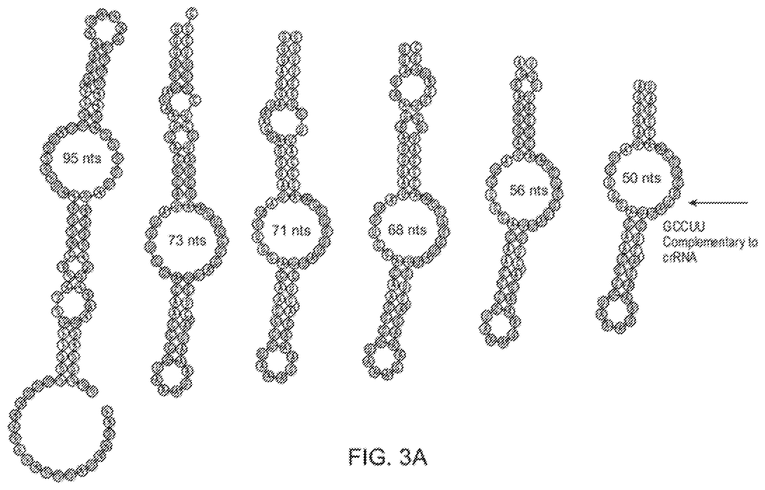

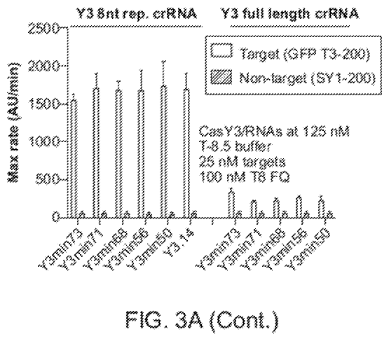

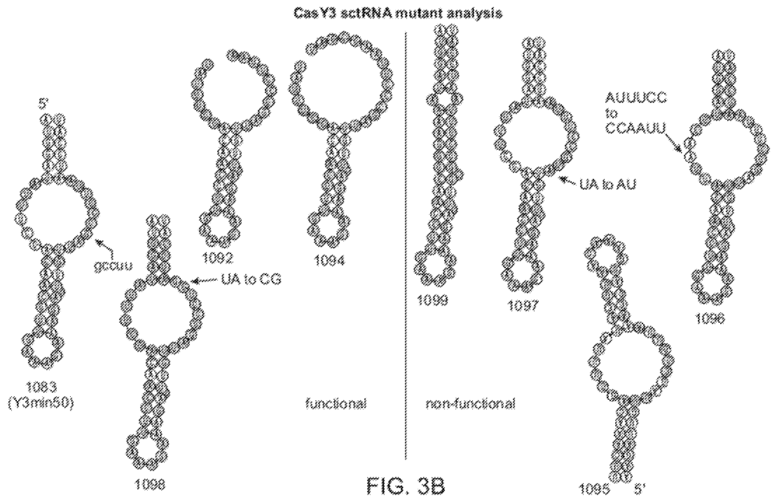

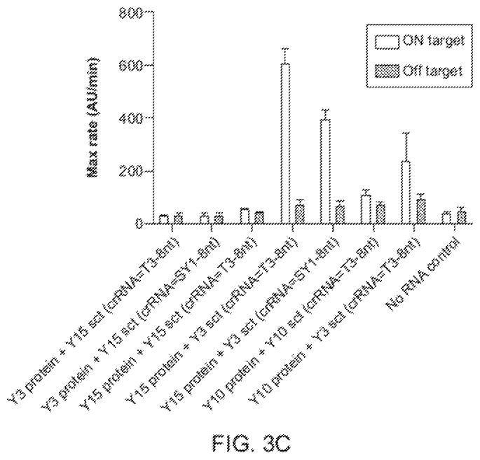

Synthesized RNAs including the GCCUU motif with various sequence 5′ and 3′ of the GCCUU sequence were tested in DETECTR assays. Functional RNP systems were reconstituted in vitro for CasY3 (SEQ ID NO: 3), CasY10 (SEQ ID NO: 9), and CasY15 (SEQ ID NO: 10) programmable nucleases. For CasY3, the intermediary RNA sequences were systematically minimized, and the sequence mutants were tested to evaluate the structure- and sequence-dependencies of the intermediary RNA. Lowest energy RNA folding tools from University of Vienna (http://rna.tbi.univie.ac.at/cgi-bin/RNAWebSuite/RNAfold.cgi) or the Mathew's lab (https://rna.urmc.rochester.edu/RNAstructureWeb/Servers/Predictl/Predictl.html) were used to examine the predicted structures of different CasY ortholog intermediary RNAs. Putative intermediary RNAs were selected based on the presence of GCCTT DNA motifs in CasY CRISPR loci. From there, intermediary RNA sequences were produced by in vitro transcription (IVT), varying the amount of sequence on each side of the GCCUU sequence. After many perturbations and identification of initial sequences that supported some level of trans cleavage activity of select CasY RNPs, a common RNA fold was identified for CasY orthologs in which the intermediary RNA GCCUU sequence was exposed in a bubble within the stem of a hair-pinned stem-loop structure (FIG. 1 and FIG. 3A-FIG. 3B). This very likely positions the GCCUU for hybridizing to the AAGGC of the crRNA repeat, without the need for strand displacement, explaining how two RNAs (crRNA and intermediary RNA) can function together in the RNP despite such limited sequence complementarity. Starting with an already trimmed down intermediary RNA sequence of 105 nucleotides roughly centered on a GGCCTT sequence within the CasY3 locus, the extraneous sequence to the basic structural motif described above was trimmed away by degrees, as shown in FIG. 3A. Trans cleavage activity was assessed in DETECTR assays with a CasY3 programmable nuclease at 125 nM, all RNA components at 125 nM including either the 8 nucleotide repeat crRNA or the full length repeat crRNA and the various minimized intermediary RNAs tested, 25 nM of the target nucleic acid, and 100 nM of a T8 FQ detector nucleic acid (SEQ ID NO: 21). FIG. 3A shows predicted structures of minimized versions of an intermediary RNA (top) and quantitation of each minimized intermediary RNA in a DETECTR reaction (bottom). The graph at the bottom of FIG. 3A shows the max rate (AU/min) for each assay condition. Assays were performed with a CasY3 programmable nuclease and either the 73 (Y3min73), 71 (Y3min71), 68 (Y3min68), 56 (Y3min56), 50 (Y3min50), or 95 (Y3.14, SEQ ID NO: 41) nucleotide crRNA show above. Each crRNA contained an 18-nucleotide spacer. The trans cleavage activity of CasY3 RNPs assembled with such minimized intermediary RNAs was unchanged relative to the longer versions for intermediary RNAs that maintained the core structure that was identified in various other orthologs (FIG. 3A, at bottom). This was the case regardless of whether a crRNA with a full length 25 nucleotide repeat or a crRNA with the optimized 8 nucleotide repeat was employed (FIG. 3A, at bottom). Thus, a minimized, core structure of the CasY3 intermediary RNA that is as effective as much larger versions was identified.

### Mutant Analysis Defined Crucial Intermediary RNA Structure and Sequence Features

Mutant analysis of CasY3 intermediary RNA was performed in order to determine the critical structural and/or sequence-specific requirements to support trans cleavage activity. FIG. 3B shows classification of the minimized intermediary RNAs of FIG. 3A as functional or non-functional. Collapsing the bubble by making the GGCCU-opposite strand complementary (FIG. 3B, RNA 1099) completely abolished CasY3 trans cleavage activity, suggesting that these 5 nucleotides that base-pair with the repeat sequence of the crRNA need to be exposed for functional RNP formation. Placing the hairpin on the opposite side of the bubble, while maintaining sequence polarities, also eliminated activity (FIG. 3B, RNA 1095), suggesting this hairpin end, as opposed to just a blunt duplex RNA end, is also recognized by a CasY3 programmable nuclease. Having established these critical structural features, mutant intermediary RNAs were created such that the overall fold remained undisrupted, to identify possible sequence-specific RNA binding by a CasY3 programmable nuclease (FIG. 3B). The data demonstrated that the sequence of the GCCUU-opposite strand of the bubble was critical for activity, even though care was taken not collapse the bubble in the predicted intermediary RNA structure (FIG. 3B, RNA 1096). Surprisingly, the two-nucleotide sequence on the same strand of the bubble adjacent 5′ to the GCCUU, 5′ AU, when mutated to 5′ UA, completely abolished activity (FIG. 3B, RNA 1097). However, mutating the bubble sequence 3′ to the GCCUU sequence did not affect the trans cleavage activity of CasY3 RNP (FIG. 3B, RNA 1098). These observations suggest that CasY3, and likely other CasY orthologs, recognize their intermediary RNA substrates by a combination of structure and sequence-specific binding.

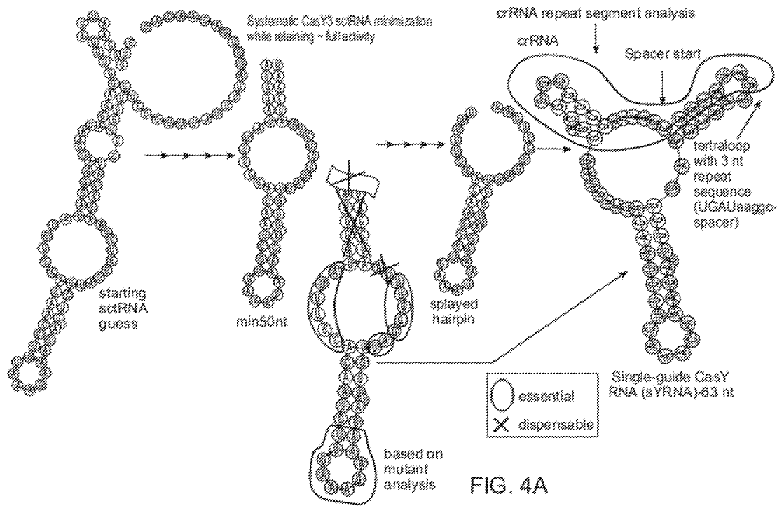

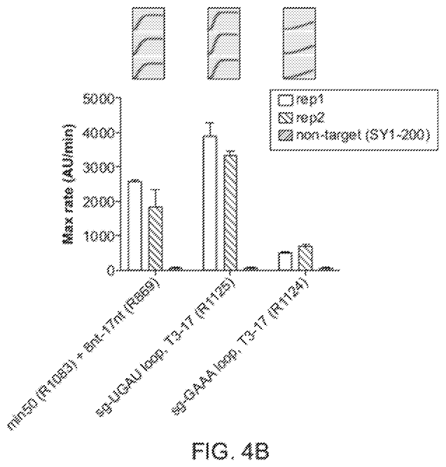

These truncation and mutation studies of CasY3 intermediary RNAs provided a fine-detailed understanding of the necessary features of the intermediary RNA and those that were dispensable (FIG. 4A). The minimal structure supporting function is revealed to be an RNA hairpin with a splayed fork of specific nucleotide sequence (FIG. 4A). This understanding enabled construction of a composite engineered guide RNA (egRNA) including a crRNA linked to an intermediary RNA.

### CasY3 Intermediary RNA Rescues Function of Orthogonal Protein Systems

The specificity of a given intermediary RNA sequence to support trans cleavage activity of different CasY orthologs was tested. Minimized intermediary RNAs from the above CasY3 experiments were tested for function with a CasY10 programmable nuclease (SEQ ID NO: 9) and a CasY15 programmable nuclease (SEQ ID NO: 10). Both CasY10 and CasY15 programmable nucleases supported target-dependent trans cleavage with the intermediary RNA and their respective 8 nucleotide repeat crRNAs (FIG. 3C). In a series of DETECTR assays, a Y3 (SEQ ID NO: 3), Y10, or Y15 programmable nucleases were incubated with crRNA, intermediary RNA, target nucleic acids, and a detector nucleic acid. The crRNAs were directed to a target nucleic acid corresponding to GFP-T3 (“T3,” SEQ ID NO: 42) or SY1 (SEQ ID NO: 119, CGCGCGTTTCGGTGATGACGGTGAAAACCTCTGACACATGCAGCTCCCGGAGACGG TCACAGCTTGTCTGTAAGCGGATGCCTGCCCGCAGACTAATCAATACCAAACTCTGG accGCGTAAACTTTCCAGTCGGGAAACCTGTCGTGCCAGCTGCATTAATGAATCGGCC AACGCGCGGGGAGAGGCGGTTTGCGTATT). Reactions were performed in the presence of 25 nM target nucleic acid, 125 nM CasY, 125 nM crRNA, 125 nM intermediary RNA, and 100 nM T8 reporter (SEQ ID NO: 21). The sequences of the crRNAs and intermediary RNAs are provided in TABLE 5. FIG. 3C shows a graph of results from DETECTR reactions with various CasY proteins in combination with various crRNA and various intermediary RNA. The graph shows the max rate (AU/min) for each assay condition. The results demonstrated that the CasY15 “native” intermediary RNA only supported trans cleavage activity for CasY15 on annealed DNA sequence targets (such as used in FIG. 2E), and not on gene fragments generated by PCR (which is used as targets in all other data figures). However, with the intermediary RNA compatible with CasY3, CasY15 programmable nucleases were activated for trans cleavage activity on gene fragment targets (FIG. 3C). An annealed target might contain ssDNA, negating the need for the non-target strand in the DNA to be displaced. This data suggested that the structure of the intermediary RNA was critical for efficient R-loop formation (heteroduplex) between the crRNA and the target strand in the duplex, thereby promoting displacement of the non-target strand.

TABLE 5
crRNA Sequences for Detection of GFP-T3 or SY1

| Sequence | | |
|---|---|---|
| Number | Description | Sequence |
| SEQ ID | Y3-GFP-T3 crRNA 8 | GAUAAGGCCAAGACCCGC |
| NO: 37 | nt repeat | GCCGAGGU |
| SEQ ID | Y3-SY1 crRNA 8 nt | GAUAAGGCAUCAAUACCA |
| NO: 43 | repeat | AACUCUGG |
| SEQ ID | Y15-GFP-T3 crRNA | AUGAAGGCCAAGACCCGC |
| NO: 44 | 8 nt repeat | GCCGAGGU |
| SEQ ID | Y15-SY1 crRNA 8 nt | AUGAAGGCAUCAAUACCA |
| NO: 45 | repeat | AACUCUGG |
| SEQ ID | Y10-GFP-T3 crRNA | AAAAAGGCCAAGACCCGC |
| NO: 46 | 8 nt repeat | GCCGAGGU |
| SEQ ID | Y10 SY1-T3 crRNA | AAAAAGGCAUCAAUACCA |
| NO: 47 | 8 nt repeat | AACUCUGG |
| SEQ ID | Y15 intermediary | CUUAGUUAAGGAUGUUCC |
| NO: 48 | RNA | AGGUUCUUUCGGGAGCCU |
| | | UGGCCUUCUCCCUUAACC |
| | | UAUGCC |
| SEQ ID | Y3 intermediary | AUGGAUAUUUCCACAAUC |
| NO: 33 | RNA (R1083) | UUGAAAGAAAGAUUUGUU |
| | | AGCCUUUAAUCCAU |

Purified CasY proteins may initially lack activity in vitro for a number of reasons, including buffer and other reaction conditions, and the sequence and/or folding of their respective RNAs. In the latter case, the activity carried over by CasY3 intermediary RNAs to other orthologs may enable their activities to be unlocked for use in developing diagnostic or gene editing RNP systems.

### Example 3

### Engineered Guide RNAs (egRNAs) for Use with a Programmable Nuclease

This example describes engineered guide RNAs (egRNA) of the present disclosure for use with a programmable nuclease disclosed herein for genome editing and detection of target nucleic acids in a sample. The elucidation of the minimal intermediary RNA structure required for trans cleavage activity by CasY3 described enabled the design of an engineered guide RNA (egRNA) for CasY3 including a crRNA linked to an intermediary RNA, and eventually other CasY orthologs. FIG. 4A shows schematics of several iterations of designs for engineering an engineered guide RNA (egRNA) and also shows the dispensable parts of the intermediary RNA structure. FIG. 4B shows a graph of results from DETECTR reactions in which various egRNAs were tested with a CasY protein. The essential parts amounted to a hair-pinned RNA with a splayed fork having strands of specific sequence. The simplicity of this structure and the fact that the GCCUU bubble need not be closed allowed the design of an egRNA as short as 63 nucleotides, well within the bounds of synthesized RNAs and significantly shorter than the ˜100 nucleotide sgRNA of Cas9. Such an egRNA would greatly simplify both in vitro and in vivo applications of CasY proteins by combining the two essential RNAs into a single functional nucleic acid.

The egRNA was designed based on the studies of the intermediary RNA structures necessary to elicit trans activity by the RNP provided in EXAMPLE 1 and EXAMPLE 2. These assays demonstrated that a hairpin RNA with a splayed fork of specific sequence was the minimal functional unit of the intermediary RNA. Fortunately, the sequence of the bubble 3′ to the GCCUU was not critical (FIG. 3B, RNA 1098), such that a splayed fork is able to accommodate a tethered crRNA on the 3′ end. Had the egRNA design initially started with a closed bubble structure, this would have necessitated a long tether between the intermediary RNA and the crRNA that (1) may not have been functional, (2) required much more optimization, (3) been longer than current RNA synthesis limits, and/or all of the above. Knowledge of the critical features of the crRNA repeat also facilitated design of the tether between the crRNA and intermediary RNA.

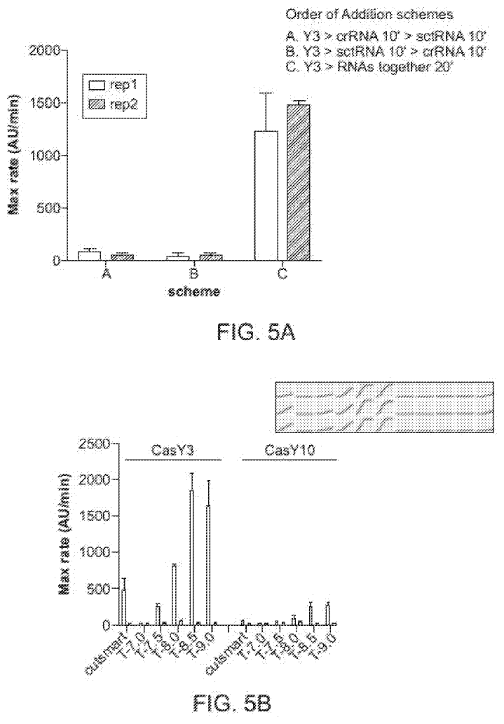

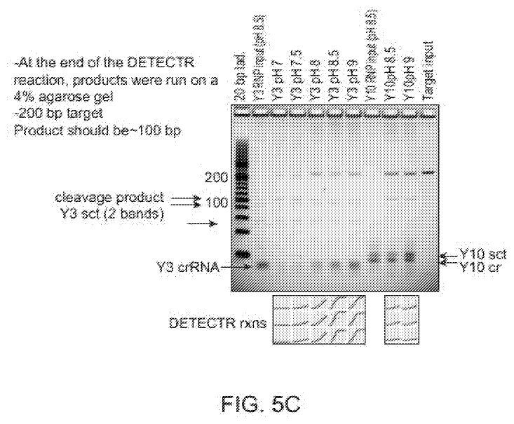

The two egRNAs designed and tested as proof of concept had a 17-nucleotide spacer against a GFP gene target, connected via a tetraloop on the 5′ end of the splayed fork minimized intermediary RNA. The first egRNA had a tetraloop with a typical, energetically favorable GAAA such as used to produce sgRNAs for Cas9. The second contains UGAU, with GAU being the first three nucleotides upstream of the AAGGC in the CasY3 crRNA repeat segment. UGAU was chosen because this gave the most stable predicted structure that incorporated the GAU repeat sequence. This egRNA was produced having knowledge from studies of the crRNA that these sequence-specific nucleotide positions immediately upstream of the AAGGC impart optimal activity. U was chosen as the 4th base in this tetraloop because it was predicted to be the most stable of the 4 possibilities in this position. This egRNA far outperformed the version that did not contain repeat sequence in these positions (˜6-fold higher trans-cleavage rate; FIG. 5B), indicating that the repeat bases within the engineered tetraloop were still recognized by CasY3. In fact, this egRNA outperformed the optimized reaction based on separate intermediary RNAs and crRNAs. This might be explained by the fact that the addition of, and presumably binding, of either RNA to CasY3 first was found to be completely inhibitory to the reaction. For an egRNA this is less of a problem, as both portions corresponding to the crRNA and intermediary RNA likely bind CasY3 at the same time since they are tethered.

### Example 4

### Assay Conditions for Programmable Nucleases

This example describes assay conditions for programmable nuclease of the present disclosure. Assay conditions for using programmable nucleases with the RNA components described herein were tested. First, the sequences in which each RNA component was added to a DETECTR reaction was evaluated. FIG. 5A shows a graph of results from DETECTR reactions in which the order of adding various components to the DETECTR reaction was modulated. In Scheme A, the CasY protein was first added, followed by the crRNA, followed by the intermediary RNA. In Scheme B, the CasY protein was first added, followed by the intermediary RNA, followed by the crRNA. In Scheme C, the CasY protein was first added, followed by both RNA components together (crRNA and intermediary RNA. The graph shows the max rate (AU/min) for each scheme that was tested. The results demonstrated that Scheme C, in which the two component RNAs were added to the reaction together, and not sequentially, produced the highest max rate. Furthermore, the results demonstrated that addition of either RNA component first to CasY3 before the other RNA component rendered the RNP completely non-functional for trans activity (FIG. 5A). DETECTR assays were performed in the presence of 125 nM intermediary RNA (R1083, SEQ ID NO: 33), 125 nM crRNA (R801, SEQ ID NO: 37), 100 nM T8 reporter (SEQ ID NO: 21), and 20 nM GFP-T3 target (SEQ ID NO: 42).

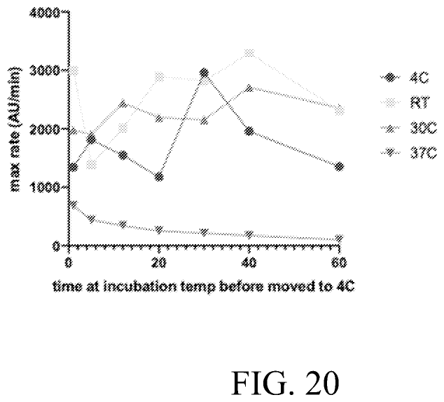

Second, the temperature of RNP assembly was investigated for its effect on the resulting RNP trans cleavage activity. CasY3 RNP was found to be thermosensitive and quickly lost activity when assembled at 37° C., however temperatures of up to 30° C. was tolerated. Interestingly this appeared only relevant to the RNP formation stage as trans cleavage activity in the presence of the target nucleic acid proceeded at a linear rate during a typical 90-minute DETECTR assay at 37° C. Thus, it is possible that the RNP is stabilized in the presence of the target nucleic acid. FIG. 20 shows the results of a DETECTR assay to test the temperature sensitivity CasY programmable nucleases. DETECTR assays were performed in the presence of 125 nM intermediary RNA (R1083, SEQ ID NO: 33), 125 nM crRNA (R801, SEQ ID NO: 37), 100 nM T8 reporter (SEQ ID NO: 21), and 20 nM GFP-T3 target (SEQ ID NO: 42). The programmable nuclease was incubated with the crRNA and the intermediary RNA at the indicated temperature and then moved to ice before performing the DETECTR assay. The results showed that CasY3 programmable nuclease tolerated temperatures up to 30° C.

Next, the impact of pH was evaluated. DETECTR assays were run under varying pH conditions for CasY3 (SEQ ID NO: 3) and CasY10 (SEQ ID NO: 9). Assays were performed in the presence of 125 nM of either CasY3 (SEQ ID NO: 3) or CasY10 (SEQ ID NO: 9) programmable nuclease in the presence of 125 nM crRNA and 125 nM intermediary RNA. The reaction was detected with 100 nM T8 reporter (SEQ ID NO: 21). The crRNA and intermediary RNA sequences are provided in TABLE 6. FIG. 5B shows a graph of results from DETECTR reactions in which the various CasY proteins were tested at several pH values. Triplicate reaction traces (time versus absorbance units) for each condition are shown below the graphed data. The graph shows the max rate (AU/min) for each scheme that was tested. The pH of the DETECTR reaction is a critical factor in activity and is held constant during RNP assembly and trans cleavage assays. CasY3 and CasY10 trans cleavage activities were both optimal at the relatively high pH ˜8.5-9, with essentially no activity at pH 7. Furthermore, they exhibited ˜6-fold enhanced trans cleavage activity from the typical biological reaction pH of 7.5 to pH 8.5 (FIG. 5B). This may prove beneficial in combination with DETECTR reactions employing pre-amplification of target nucleic acids using isothermal amplification via LAMP, which utilizes a buffer with a pH of 8.8. With the same optimal pH, this can simplify DETECTR-based DNA detection device design by potentially eliminating the need to adjust, change, and/or dilute buffers as part of any device fluidics.

TABLE 6
crRNAs and Intermediary RNA

| SEQ ID | | | |
|---|---|---|---|
| NO: | Component | Name | Sequence |
| SEQ ID | Intermediary | Y3.14 | UCGGGAGGAUAAGUAUG |
| NO: 41 | RNA | | GAUAUUUCCACAAUCUU |
| | | | GAAAGAAAGAUUUGUUA |
| | | | GCCUUUAAUCCAUUCUC |
| | | | CUUUCCCUUUAUUUUAU |
| | | | CUGACAACAU |
| SEQ ID | crRNA | R801 | GAUAAGGCCAAGACCCG |
| NO: 37 | | | CGCCGAGGU |
| SEQ ID | crRNA | Y10.5 | UGGUUCCAUUCUCCUGA |
| NO: 49 | | | GCUCCGUUGAGAGCGAG |
| | | | AAAGAGAACUAGCCUUC |
| | | | CCACUCAUCACUCCGGC |
| | | | AUAUUCU |
| SEQ ID | crRNA | R815 | AAAAAGGCCAAGACCCG |
| NO: 46 | | | CGCCGAGGU |

After CasY3 and CasY10 DETECTR reactions were performed in different pH buffers, assay plate wells were analyzed for the extent of cis cleavage that had occurred. FIG. 5C shows an agarose gel of DETECTR assay products, revealing the extent of cis cleavage in the DETECTR reactions. Various nucleic acid species in the reaction are labeled. Triplicate reaction traces (time versus absorbance units) for each condition are shown below the graphed data. While trans cleavage activity increased along with reaction pH, the cis cleavage activity observed followed an inverse pattern. This suggests that cis and trans cleavage are separate activities and provides evidence that it is not necessarily the case that the CasY protein first makes a cis cleavage and only then unleashes indiscriminate trans cleavage nuclease activity. From an applications point of view, pH is a simple change to the reaction condition that can modulate cis versus trans cleavage nuclease activity, depending on which is desired. For example, at pH 7.0, CasY3 cis cleavage was observed without detectable trans cleavage (FIG. 5C).

### Example 5

### Genome Editing with CasY Programmable Nucleases and egRNA Systems

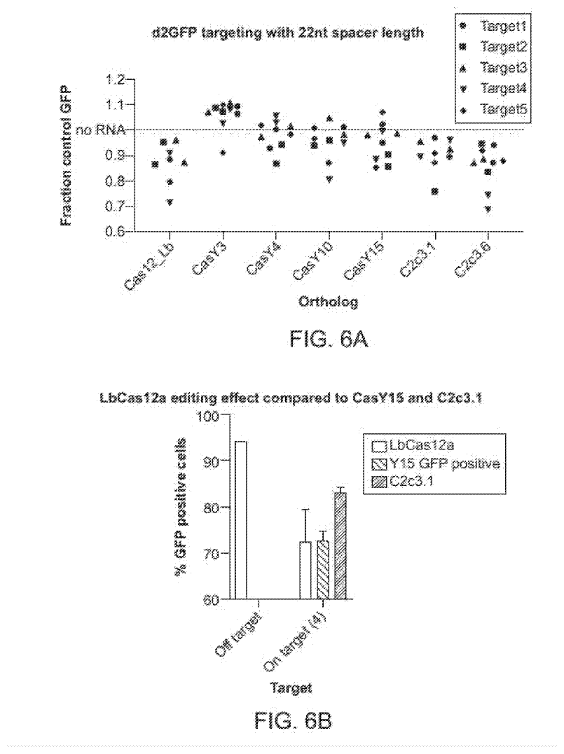

This example describes genome editing with CasY programmable nucleases and egRNA systems of the present disclosure. The ability for various programmable nuclease, including CasY, to edit HEK293T cells was investigated. HEK293T cells were transfected with a DNA plasmid and PCR product was used to encode RNA targeting the d2GFP portion of HEK293T cell. These two pieces of DNA were transfected into the cells using lipid-based transfection and observed 90 hours post-transfection by flow cytometry. The extent of editing was measured by the amount of fraction of cells that still fluoresced in the GFP channel. CasY results were compared against those for LbCas12a with both biological and technical replicates. FIG. 6A shows results from genome editing with various programmable nucleases targeting a GFP domain. The graphed results show the fraction of cells that still fluoresced in the GFP channel, as determined by flow cytometry, after the GFP domain was targeted with the various programmable nucleases tested. FIG. 6B shows results from a comparison of genome editing efficiency of an LbCas12a protein to a CasY protein and a c2c3 protein by measuring the percentage of cells that still fluorescence in the GFP channel, as determined by flow cytometry, after the GFP domain was targeted with the various programmable nucleases tested. The results demonstrated that the editing effects of some CasY proteins were similar to that of LbCas12a and can be further optimized now with the design of the egRNA and its optimized characteristics.

### Example 6

### Bioproduction Using CasY Programmable Nucleases and egRNA Systems

This example describes bioproduction using CasY programmable nucleases and egRNA systems of the present disclosure.

Competent bacterial cells are transformed with plasmids encoding a CasY protein, an engineered guide RNA (egRNA) system, and a donor nucleic acid. The egRNA has crRNA with a spacer region that hybridizes to a region of the bacterial genome. The donor nucleic acid includes an inducible promoter sequence and a sequence encoding a therapeutic peptide. The CasY protein is expressed in the transformed bacteria. The egRNA system is transcribed in the transformed bacteria. The expressed CasY protein complexes with the transcribed egRNA system and is directed to the region of the bacterial genome. Cis cleavage activity of the CasY protein is activated upon recruitment of the CasY-egRNA RNP complex to the region of the bacterial genome. The activated CasY protein cleaves the region of the bacterial genome. The donor nucleic acid is incorporated into the bacterial genome by non-homologous end joining at the site of cleavage. The therapeutic peptide is expressed in the bacterial cell following induction of the inducible promoter.

### Example 7

### Genetic Modification Using CasY Programmable Nucleases and egRNA Systems

This example describes genetic modification using CasY programmable nucleases and egRNA systems of the present disclosure.

Plant cells are transformed with plasmids encoding a CasY protein, an engineered guide RNA (egRNA) system, and a donor nucleic acid. The egRNA has crRNA with a spacer region that hybridizes to a region of the plant genome. The donor nucleic acid includes a promoter sequence and a sequence encoding an insecticidal protein. The CasY protein is expressed in the transformed plant cell. The egRNA system is transcribed in the transformed plant cell. The expressed CasY protein complexes with the transcribed egRNA system and is directed to the region of the plant genome. Cis cleavage activity of the CasY protein is activated upon recruitment of the CasY-egRNA RNP complex to the region of the plant genome. The activated CasY protein cleaves the region of the plant genome. The donor nucleic acid is incorporated into the plant genome by non-homologous end joining at the site of cleavage. The insecticidal protein is expressed in the plant cell following, thereby increasing the insect resistance of the plant.

### Example 8

### In Vitro Diagnostics Using CasY Programmable Nucleases and egRNA Systems

This example describes in vitro diagnostics using CasY programmable nucleases and egRNA systems of the present disclosure.

A saliva sample collected from a patient to be diagnosed is contacted with a CasY programmable nuclease, an egRNA system, and a detector nucleic acid. The egRNA system has a crRNA with a spacer region that hybridizes to a region of a nucleotide sequence of an infectious agent. The detector nucleic acid comprises a single stranded DNA and a detection moiety. The CasY programmable nuclease complexes with the egRNA system. If the infectious agent is present in the saliva sample, the CasY-egRNA RNP complex binds to the region of the nucleotide sequence of the infectious agent. Trans cleavage activity of the CasY protein is activated upon binding of the CasY-egRNA RNP complex to the region of the nucleotide sequence of the infectious agent, and the activated CasY cleaves the detector nucleic acid. The cleaved detector nucleic acid produces a detectable signal, indicating that the patient to be diagnosed is positive for the infectious agent.

### Example 9

### Distinguishing Two Single Nucleotide Polymorphisms in PNPLA3 Using DETECTR

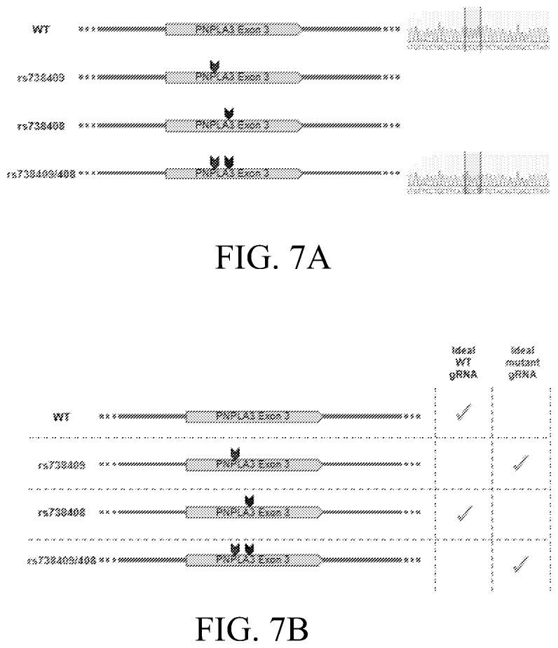

This example describes using DETECTR to distinguish two single nucleotide polymorphisms in PNPLA3. The PNPLA3 gene contains two SNP sites separated by only two nucleotide bases. FIG. 7A illustrates genetic variations in exon 3 of the patatin-like phospholipase domain-containing protein 3 (PNPLA3) gene. A first single nucleotide mutation (rs738409) leads to a I148M amino acid substitution associated with an increased risk of nonalcoholic fatty liver disease. A second single nucleotide mutation (rs738408) codes a silent mutation with a 70% linkage to the at-risk allele. There are nine possible genetic combinations of wild type (“WT”), at-risk mutant (rs738409), and non-risk mutant (rs738408) alleles.

Guide nucleic acids were designed to distinguish the at-risk allele from the non-risk allele and the wild type sequence using a DETECTR assay. FIG. 7B illustrates detection of PNPLA3 alleles using gRNAs to detect the presence or absence of the at-risk allele (rs738409) while ignoring the non-risk allele (rs738408). The wild type (“WT”) gRNA detects WT or non-risk alleles lacking the at-risk allele, and the mutant gRNA detects the at-risk allele with or without the non-risk allele.

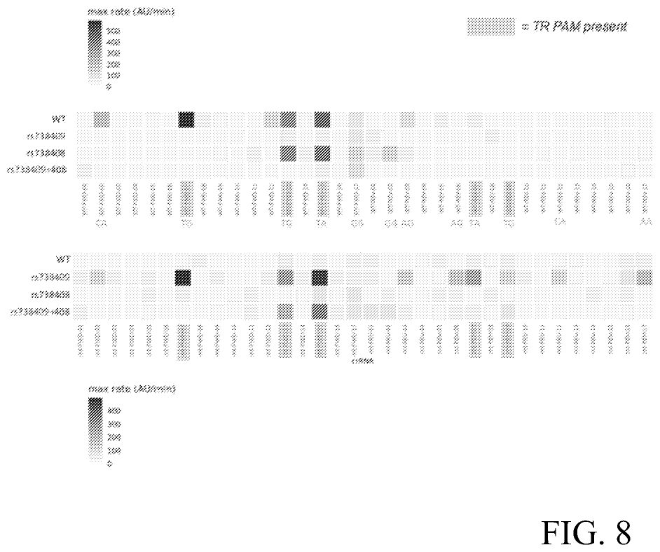

Composite egRNAs compatible with a CasY programmable nuclease were designed to detect the PNPLA3 SNPs. Composite egRNAs with spacers targeted to the at-risk SNP at different positions relative to the 5′ end of the spacer were tested. The sequences of the composite egRNAs are provided in TABLE 7. FIG. 8 shows the maximum rates (fluorescence detected per minute) of a DETECTR assay detecting wild type (“WT”), at-risk (rs738409), non-risk (rs738409), or both at-risk and non-risk (rs738409+408) alleles of PNPLA3 using different composite egRNAs. Samples were detected using CasY3 (SEQ ID NO: 3). Shaded egRNAs denote egRNAs directed to sequences containing a TR PAM site.

TABLE 7
Composite egRNAs for Detection of PNPLA3 SNPs

| SEQ ID | | | Position | |
|---|---|---|---|---|
| NO: | Name | Target | of SNP | Sequence |
| SEQ ID | PNPLA3- | WT | 1 | UAUUUCCACAAUCUUGAAAGAAAGA |
| NO: 50 | WT-F01 | PNPLA3 | | UUGUUAGCCUUUGAUAAGGCCCCCU |
| | | | | UCUACAGUGGCC |
| SEQ ID | PNPLA3- | WT | 2 | UAUUUCCACAAUCUUGAAAGAAAGA |
| NO: 51 | WT-F02 | PNPLA3 | | UUGUUAGCCUUUGAUAAGGCUCCCC |
| | | | | UUCUACAGUGGC |
| SEQ ID | PNPLA3- | WT | 3 | UAUUUCCACAAUCUUGAAAGAAAGA |
| NO: 52 | WT-F03 | PNPLA3 | | UUGUUAGCCUUUGAUAAGGCAUCCC |
| | | | | CUUCUACAGUGG |
| SEQ ID | PNPLA3- | WT | 4 | UAUUUCCACAAUCUUGAAAGAAAGA |
| NO: 53 | WT-F04 | PNPLA3 | | UUGUUAGCCUUUGAUAAGGCCAUCC |
| | | | | CCUUCUACAGUG |
| SEQ ID | PNPLA3- | WT | 5 | UAUUUCCACAAUCUUGAAAGAAAGA |
| NO: 54 | WT-F05 | PNPLA3 | | UUGUUAGCCUUUGAUAAGGCUCAUC |
| | | | | CCCUUCUACAGU |
| SEQ ID | PNPLA3- | WT | 6 | UAUUUCCACAAUCUUGAAAGAAAGA |
| NO: 55 | WT-F06 | PNPLA3 | | UUGUUAGCCUUUGAUAAGGCUUCAU |
| | | | | CCCCUUCUACAG |
| SEQ ID | PNPLA3- | WT | 7 | UAUUUCCACAAUCUUGAAAGAAAGA |
| NO: 56 | WT-F07 | PNPLA3 | | UUGUUAGCCUUUGAUAAGGCCUUCA |
| | | | | UCCCCUUCUACA |
| SEQ ID | PNPLA3- | WT | 8 | UAUUUCCACAAUCUUGAAAGAAAGA |
| NO: 57 | WT-F08 | PNPLA3 | | UUGUUAGCCUUUGAUAAGGCGCUUC |
| | | | | AUCCCCUUCUAC |
| SEQ ID | PNPLA3- | WT | 9 | UAUUUCCACAAUCUUGAAAGAAAGA |
| NO: 58 | WT-F09 | PNPLA3 | | UUGUUAGCCUUUGAUAAGGCUGCUU |
| | | | | CAUCCCCUUCUA |
| SEQ ID | PNPLA3- | WT | 10 | UAUUUCCACAAUCUUGAAAGAAAGA |
| NO: 59 | WT-F10 | PNPLA3 | | UUGUUAGCCUUUGAUAAGGCCUGCU |
| | | | | UCAUCCCCUUCU |
| SEQ ID | PNPLA3- | WT | 11 | UAUUUCCACAAUCUUGAAAGAAAGA |
| NO: 60 | WT-F11 | PNPLA3 | | UUGUUAGCCUUUGAUAAGGCCCUGC |
| | | | | UUCAUCCCCUUC |
| SEQ ID | PNPLA3- | WT | 12 | UAUUUCCACAAUCUUGAAAGAAAGA |
| NO: 61 | WT-F12 | PNPLA3 | | UUGUUAGCCUUUGAUAAGGCUCCUG |
| | | | | CUUCAUCCCCUU |
| SEQ ID | PNPLA3- | WT | 13 | UAUUUCCACAAUCUUGAAAGAAAGA |
| NO: 62 | WT-F13 | PNPLA3 | | UUGUUAGCCUUUGAUAAGGCUUCCU |
| | | | | GCUUCAUCCCCU |
| SEQ ID | PNPLA3- | WT | 14 | UAUUUCCACAAUCUUGAAAGAAAGA |
| NO: 63 | WT-F14 | PNPLA3 | | UUGUUAGCCUUUGAUAAGGCGUUCC |
| | | | | UGCUUCAUCCCC |
| SEQ ID | PNPLA3- | WT | 15 | UAUUUCCACAAUCUUGAAAGAAAGA |
| NO: 64 | WT-F15 | PNPLA3 | | UUGUUAGCCUUUGAUAAGGCUGUUC |
| | | | | CUGCUUCAUCCC |
| SEQ ID | PNPLA3- | WT | 16 | UAUUUCCACAAUCUUGAAAGAAAGA |
| NO: 65 | WT-F16 | PNPLA3 | | UUGUUAGCCUUUGAUAAGGCAUGUU |
| | | | | CCUGCUUCAUCC |
| SEQ ID | PNPLA3- | WT | 17 | UAUUUCCACAAUCUUGAAAGAAAGA |
| NO: 66 | WT-F17 | PNPLA3 | | UUGUUAGCCUUUGAUAAGGCUAUGU |
| | | | | UCCUGCUUCAUC |
| SEQ ID | PNPLA3- | WT | 1 | UAUUUCCACAAUCUUGAAAGAAAGA |
| NO: 67 | WT-R01 | PNPLA3 | | UUGUUAGCCUUUGAUAAGGCGAUGA |
| | | | | AGCAGGAACAUA |
| SEQ ID | PNPLA3- | WT | 2 | UAUUUCCACAAUCUUGAAAGAAAGA |
| NO: 68 | WT-R02 | PNPLA3 | | UUGUUAGCCUUUGAUAAGGCGGAUG |
| | | | | AAGCAGGAACAU |
| SEQ ID | PNPLA3- | WT | 3 | UAUUUCCACAAUCUUGAAAGAAAGA |
| NO: 69 | WT-R03 | PNPLA3 | | UUGUUAGCCUUUGAUAAGGCGGGAU |
| | | | | GAAGCAGGAACA |
| SEQ ID | PNPLA3- | WT | 4 | UAUUUCCACAAUCUUGAAAGAAAGA |
| NO: 70 | WT-R04 | PNPLA3 | | UUGUUAGCCUUUGAUAAGGCGGGGA |
| | | | | UGAAGCAGGAAC |
| SEQ ID | PNPLA3- | WT | 5 | UAUUUCCACAAUCUUGAAAGAAAGA |
| NO: 71 | WT-R05 | PNPLA3 | | UUGUUAGCCUUUGAUAAGGCAGGGG |
| | | | | AUGAAGCAGGAA |
| SEQ ID | PNPLA3- | WT | 6 | UAUUUCCACAAUCUUGAAAGAAAGA |
| NO: 72 | WT-R06 | PNPLA3 | | UUGUUAGCCUUUGAUAAGGCAAGGG |
| | | | | GAUGAAGCAGGA |
| SEQ ID | PNPLA3- | WT | 7 | UAUUUCCACAAUCUUGAAAGAAAGA |
| NO: 73 | WT-R07 | PNPLA3 | | UUGUUAGCCUUUGAUAAGGCGAAGG |
| | | | | GGAUGAAGCAGG |
| SEQ ID | PNPLA3- | WT | 8 | UAUUUCCACAAUCUUGAAAGAAAGA |
| NO: 74 | WT-R08 | PNPLA3 | | UUGUUAGCCUUUGAUAAGGCAGAAG |
| | | | | GGGAUGAAGCAG |
| SEQ ID | PNPLA3- | WT | 9 | UAUUUCCACAAUCUUGAAAGAAAGA |
| NO: 75 | WT-R09 | PNPLA3 | | UUGUUAGCCUUUGAUAAGGCUAGAA |
| | | | | GGGGAUGAAGCA |
| SEQ ID | PNPLA3- | WT | 10 | UAUUUCCACAAUCUUGAAAGAAAGA |
| NO: 76 | WT-R10 | PNPLA3 | | UUGUUAGCCUUUGAUAAGGCGUAGA |
| | | | | AGGGGAUGAAGC |
| SEQ ID | PNPLA3- | WT | 11 | UAUUUCCACAAUCUUGAAAGAAAGA |
| NO: 77 | WT-R11 | PNPLA3 | | UUGUUAGCCUUUGAUAAGGCUGUAG |
| | | | | AAGGGGAUGAAG |
| SEQ ID | PNPLA3- | WT | 12 | UAUUUCCACAAUCUUGAAAGAAAGA |
| NO: 78 | WT-R12 | PNPLA3 | | UUGUUAGCCUUUGAUAAGGCCUGUA |
| | | | | GAAGGGGAUGAA |
| SEQ ID | PNPLA3- | WT | 13 | UAUUUCCACAAUCUUGAAAGAAAGA |
| NO: 79 | WT-R13 | PNPLA3 | | UUGUUAGCCUUUGAUAAGGCACUGU |
| | | | | AGAAGGGGAUGA |
| SEQ ID | PNPLA3- | WT | 14 | UAUUUCCACAAUCUUGAAAGAAAGA |
| NO: 80 | WT-R14 | PNPLA3 | | UUGUUAGCCUUUGAUAAGGCCACUG |
| | | | | UAGAAGGGGAUG |
| SEQ ID | PNPLA3- | WT | 15 | UAUUUCCACAAUCUUGAAAGAAAGA |
| NO: 81 | WT-R15 | PNPLA3 | | UUGUUAGCCUUUGAUAAGGCCCACU |
| | | | | GUAGAAGGGGAU |
| SEQ ID | PNPLA3- | WT | 16 | UAUUUCCACAAUCUUGAAAGAAAGA |
| NO: 82 | WT-R16 | PNPLA3 | | UUGUUAGCCUUUGAUAAGGCGCCAC |
| | | | | UGUAGAAGGGGA |
| SEQ ID | PNPLA3- | WT | 17 | UAUUUCCACAAUCUUGAAAGAAAGA |
| NO: 83 | WT-R17 | PNPLA3 | | UUGUUAGCCUUUGAUAAGGCGGCCA |
| | | | | CUGUAGAAGGGG |
| SEQ ID | PNPLA3- | I148M | 1 | UAUUUCCACAAUCUUGAAAGAAAGA |
| NO: 84 | mutant- | PNPLA3 | | UUGUUAGCCUUUGAUAAGGCGCCCU |
| | F01 | | | UCUACAGUGGCC |
| SEQ ID | PNPLA3- | I148M | 2 | UAUUUCCACAAUCUUGAAAGAAAGA |
| NO: 85 | mutant- | PNPLA3 | | UUGUUAGCCUUUGAUAAGGCUGCCC |
| | F02 | | | UUCUACAGUGGC |
| SEQ ID | PNPLA3- | I148M | 3 | UAUUUCCACAAUCUUGAAAGAAAGA |
| NO: 86 | mutant- | PNPLA3 | | UUGUUAGCCUUUGAUAAGGCAUGCC |
| | F03 | | | CUUCUACAGUGG |
| SEQ ID | PNPLA3- | I148M | 4 | UAUUUCCACAAUCUUGAAAGAAAGA |
| NO: 87 | mutant- | PNPLA3 | | UUGUUAGCCUUUGAUAAGGCCAUGC |
| | F04 | | | CCUUCUACAGUG |
| SEQ ID | PNPLA3- | I148M | 5 | UAUUUCCACAAUCUUGAAAGAAAGA |
| NO: 88 | mutant- | PNPLA3 | | UUGUUAGCCUUUGAUAAGGCUCAUG |
| | F05 | | | CCCUUCUACAGU |
| SEQ ID | PNPLA3- | I148M | 6 | UAUUUCCACAAUCUUGAAAGAAAGA |
| NO: 89 | mutant- | PNPLA3 | | UUGUUAGCCUUUGAUAAGGCUUCAU |
| | F06 | | | GCCCUUCUACAG |
| SEQ ID | PNPLA3- | I148M | 7 | UAUUUCCACAAUCUUGAAAGAAAGA |
| NO: 90 | mutant- | PNPLA3 | | UUGUUAGCCUUUGAUAAGGCCUUCA |
| | F07 | | | UGCCCUUCUACA |
| SEQ ID | PNPLA3- | I148M | 8 | UAUUUCCACAAUCUUGAAAGAAAGA |
| NO: 91 | mutant- | PNPLA3 | | UUGUUAGCCUUUGAUAAGGCGCUUC |
| | F08 | | | AUGCCCUUCUAC |
| SEQ ID | PNPLA3- | I148M | 9 | UAUUUCCACAAUCUUGAAAGAAAGA |
| NO: 92 | mutant- | PNPLA3 | | UUGUUAGCCUUUGAUAAGGCUGCUU |
| | F09 | | | CAUGCCCUUCUA |
| SEQ ID | PNPLA3- | I148M | 10 | UAUUUCCACAAUCUUGAAAGAAAGA |
| NO: 93 | mutant- | PNPLA3 | | UUGUUAGCCUUUGAUAAGGCCUGCU |
| | F10 | | | UCAUGCCCUUCU |
| SEQ ID | PNPLA3- | I148M | 11 | UAUUUCCACAAUCUUGAAAGAAAGA |
| NO: 94 | mutant- | PNPLA3 | | UUGUUAGCCUUUGAUAAGGCCCUGC |
| | F11 | | | UUCAUGCCCUUC |
| SEQ ID | PNPLA3- | I148M | 12 | UAUUUCCACAAUCUUGAAAGAAAGA |
| NO: 95 | mutant- | PNPLA3 | | UUGUUAGCCUUUGAUAAGGCUCCUG |
| | F12 | | | CUUCAUGCCCUU |
| SEQ ID | PNPLA3- | I148M | 13 | UAUUUCCACAAUCUUGAAAGAAAGA |
| NO: 96 | mutant- | PNPLA3 | | UUGUUAGCCUUUGAUAAGGCUUCCU |
| | F13 | | | GCUUCAUGCCCU |
| SEQ ID | PNPLA3- | I148M | 14 | UAUUUCCACAAUCUUGAAAGAAAGA |
| NO: 97 | mutant- | PNPLA3 | | UUGUUAGCCUUUGAUAAGGCGUUCC |
| | F14 | | | UGCUUCAUGCCC |
| SEQ ID | PNPLA3- | I148M | 15 | UAUUUCCACAAUCUUGAAAGAAAGA |
| NO: 98 | mutant- | PNPLA3 | | UUGUUAGCCUUUGAUAAGGCUGUUC |
| | F15 | | | CUGCUUCAUGCC |
| SEQ ID | PNPLA3- | I148M | 16 | UAUUUCCACAAUCUUGAAAGAAAGA |
| NO: 99 | mutant- | PNPLA3 | | UUGUUAGCCUUUGAUAAGGCAUGUU |
| | F16 | | | CCUGCUUCAUGC |
| SEQ ID | PNPLA3- | I148M | 17 | UAUUUCCACAAUCUUGAAAGAAAGA |
| NO: 100 | mutant- | PNPLA3 | | UUGUUAGCCUUUGAUAAGGCUAUGU |
| | F17 | | | UCCUGCUUCAUG |
| SEQ ID | PNPLA3- | I148M | 1 | UAUUUCCACAAUCUUGAAAGAAAGA |
| NO: 101 | mutant- | PNPLA3 | | UUGUUAGCCUUUGAUAAGGCCAUGA |
| | R01 | | | AGCAGGAACAUA |
| SEQ ID | PNPLA3- | I148M | 2 | UAUUUCCACAAUCUUGAAAGAAAGA |
| NO: 102 | mutant- | PNPLA3 | | UUGUUAGCCUUUGAUAAGGCGCAUG |
| | R02 | | | AAGCAGGAACAU |
| SEQ ID | PNPLA3- | I148M | 3 | UAUUUCCACAAUCUUGAAAGAAAGA |
| NO: 103 | mutant- | PNPLA3 | | UUGUUAGCCUUUGAUAAGGCGGCAU |
| | R03 | | | GAAGCAGGAACA |
| SEQ ID | PNPLA3- | I148M | 4 | UAUUUCCACAAUCUUGAAAGAAAGA |
| NO: 104 | mutant- | PNPLA3 | | UUGUUAGCCUUUGAUAAGGCGGGCA |
| | R04 | | | UGAAGCAGGAAC |
| SEQ ID | PNPLA3- | I148M | 5 | UAUUUCCACAAUCUUGAAAGAAAGA |
| NO: 105 | mutant- | PNPLA3 | | UUGUUAGCCUUUGAUAAGGCAGGGC |
| | R05 | | | AUGAAGCAGGAA |
| SEQ ID | PNPLA3- | I148M | 6 | UAUUUCCACAAUCUUGAAAGAAAGA |
| NO: 106 | mutant- | PNPLA3 | | UUGUUAGCCUUUGAUAAGGCAAGGG |
| | R06 | | | CAUGAAGCAGGA |
| SEQ ID | PNPLA3- | I148M | 7 | UAUUUCCACAAUCUUGAAAGAAAGA |
| NO: 107 | mutant- | PNPLA3 | | UUGUUAGCCUUUGAUAAGGCGAAGG |
| | R07 | | | GCAUGAAGCAGG |
| SEQ ID | PNPLA3- | I148M | 8 | UAUUUCCACAAUCUUGAAAGAAAGA |
| NO: 108 | mutant- | PNPLA3 | | UUGUUAGCCUUUGAUAAGGCAGAAG |
| | R08 | | | GGCAUGAAGCAG |
| SEQ ID | PNPLA3- | I148M | 9 | UAUUUCCACAAUCUUGAAAGAAAGA |
| NO: 109 | mutant- | PNPLA3 | | UUGUUAGCCUUUGAUAAGGCUAGAA |
| | R09 | | | GGGCAUGAAGCA |
| SEQ ID | PNPLA3- | I148M | 10 | UAUUUCCACAAUCUUGAAAGAAAGA |
| NO: 110 | mutant- | PNPLA3 | | UUGUUAGCCUUUGAUAAGGCGUAGA |
| | R10 | | | AGGGCAUGAAGC |
| SEQ ID | PNPLA3- | I148M | 11 | UAUUUCCACAAUCUUGAAAGAAAGA |
| NO: 111 | mutant- | PNPLA3 | | UUGUUAGCCUUUGAUAAGGCUGUAG |
| | R11 | | | AAGGGCAUGAAG |
| SEQ ID | PNPLA3- | I148M | 12 | UAUUUCCACAAUCUUGAAAGAAAGA |
| NO: 112 | mutant- | PNPLA3 | | UUGUUAGCCUUUGAUAAGGCCUGUA |
| | R12 | | | GAAGGGCAUGAA |
| SEQ ID | PNPLA3- | I148M | 13 | UAUUUCCACAAUCUUGAAAGAAAGA |
| NO: 113 | mutant- | PNPLA3 | | UUGUUAGCCUUUGAUAAGGCACUGU |
| | R13 | | | AGAAGGGCAUGA |
| SEQ ID | PNPLA3- | I148M | 14 | UAUUUCCACAAUCUUGAAAGAAAGA |
| NO: 114 | mutant- | PNPLA3 | | UUGUUAGCCUUUGAUAAGGCCACUG |
| | R14 | | | UAGAAGGGCAUG |
| SEQ ID | PNPLA3- | I148M | 15 | UAUUUCCACAAUCUUGAAAGAAAGA |
| NO: 115 | mutant- | PNPLA3 | | UUGUUAGCCUUUGAUAAGGCCCACU |
| | R15 | | | GUAGAAGGGCAU |
| SEQ ID | PNPLA3- | I148M | 16 | UAUUUCCACAAUCUUGAAAGAAAGA |
| NO: 116 | mutant- | PNPLA3 | | UUGUUAGCCUUUGAUAAGGCGCCAC |
| | R16 | | | UGUAGAAGGGCA |
| SEQ ID | PNPLA3- | I148M | 17 | UAUUUCCACAAUCUUGAAAGAAAGA |
| NO: 117 | mutant- | PNPLA3 | | UUGUUAGCCUUUGAUAAGGCGGCCA |
| | R17 | | | CUGUAGAAGGGC |

Several composite egRNAs were identified that were capable of detecting the presence of the at-risk allele while ignoring the non-risk allele. In particular mt-FWD-13 (SEQ ID NO: 96) and mt-FWD-15 (SEQ ID NO: 98) were capable of detecting the presence of the at-risk allele while ignoring the non-risk allele. Several composite egRNAs were identified that were capable of detecting the wild type sequence and the non-risk allele absence of the at-risk mutation. In particular, WT-FWD-13 (SEQ ID NO: 62) and WT-FWD-15 (SEQ ID NO: 64) were capable of detecting the wild type sequence and the non-risk allele absence of the at-risk mutation.

### Example 10

### Pooled gRNAs to Distinguish Two Single Nucleotide Polymorphisms in PNPLA3

This example describes pooled gRNAs to distinguish two single nucleotide polymorphisms in PNPLA3. Guide RNAs identified in EXAMPLE 9 that are specific for a single PNPLA3 allele are pooled for detection of at-risk alleles. In a first assay, gRNAs are tested individually to confirm specificity of each gRNA for the targeted SNP combination. Samples were detected using a CasY programmable nuclease. NTC denotes a negative control lacking a target nucleic acid.

Guide RNAs directed to the WT allele and the rs738408 allele are then pooled for detection of the WT allele and the non-risk allele in the absence of the at-risk allele. Guide RNAs directed to the rs738409 allele and the rs738409+408 allele are pooled for the detection of the at-risk allele independent of the presence or absence of the non-risk allele. Pools of gRNA are designed to detect the wild type or non-risk alleles or at-risk allele independent of the presence or absence of the non-risk allele. Samples are detected using a CasY programmable nuclease. NTC denotes a negative control lacking a target nucleic acid. The results showed that pooled gRNAs were capable of detecting combinations of SNPs.

### Example 11

### Screening of Pre-Amplification Conditions for Rapid Detection of a Target Nucleic Acid

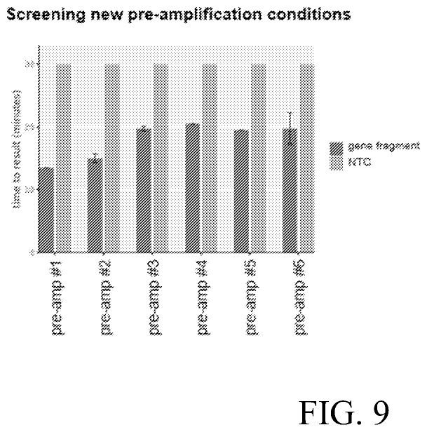

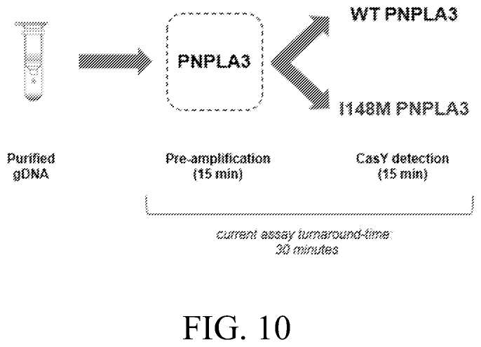

This example describes screening of pre-amplification conditions for rapid detection of a target nucleic acid. Six different amplification conditions were tested on samples containing either a target gene fragment or no target. FIG. 9 shows the time to result (minutes) of a DETECTR assay using different pre-amplification conditions (“pre-amp #1” through “pre-amp #5”). Time to result was measured as the time at which exponential amplification occurs. Variation of pre-amplification conditions enabled pre-amplification times of less than 15 minutes. NTC denotes a negative control lacking a target nucleic acid. The results show that select amplification conditions (pre-amp #1) enabled amplification of a target nucleic acid in less than 15 minutes. Amplification of the target in less than 15 minutes enabled detection of the target nucleic acid in about 30 minutes. FIG. 10 illustrates an assay workflow for detecting at-risk alleles of a target gene in about 30 minutes using a CasY programmable nuclease. A sample, for example purified genomic DNA (“gDNA”), undergoes pre-amplification for about 15 minutes followed by detection with a programmable nuclease, for example a CasY programmable nuclease, for about 15 minutes.

### Example 12

### Limit of Detection of a DETECTR Reaction Performed in Under 30 Minutes

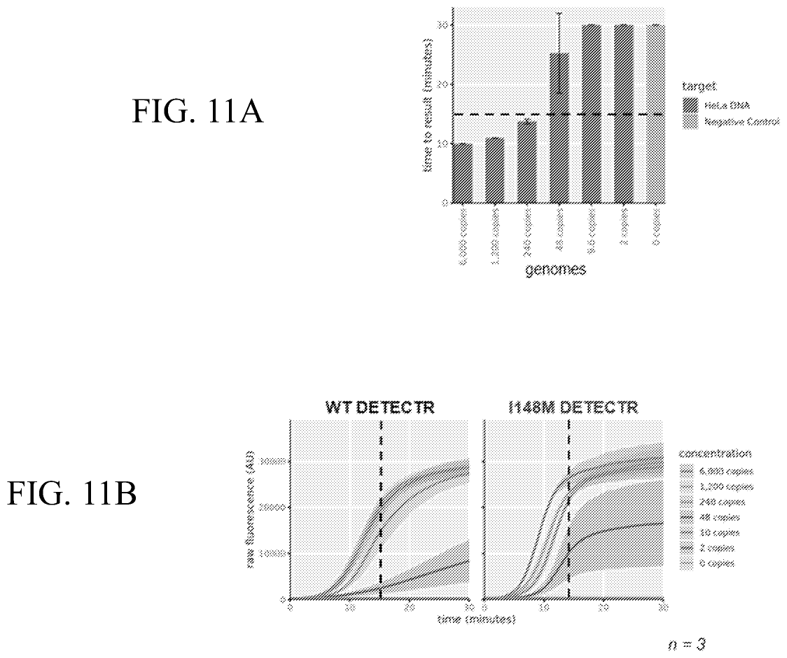

This example describes the limit of detection of a DETECTR reaction performed in under 30 minutes. Samples containing serial dilutions of HeLa DNA target nucleic acid were tested using a DETECTR assay. FIG. 11A shows limit of detection of a DETECTR assay in the presence of decreasing number of copies of genomic DNA (“HeLa DNA”) per reaction. Samples containing 240 copies of genomic DNA per reaction could be detected in less than 30 minutes. FIG. 11B shows the limit of detection of a DETECTR assays to detect a wild type (left) or at-risk (right) allele of PNPLA3 in the presence of decreasing copies of DNA (“concentration”) per reaction. Samples containing 240 copies of genomic DNA per reaction could be detected in less than 30 minutes (indicated by vertical dashed lines). Together, these results showed that a target nucleic acid can be detected in under 30 minutes at concentrations of as low as about 240 genome copies per reaction.

### Example 13

### Detection of at-Risk PNPLA3 Alleles in Heterozygous Samples

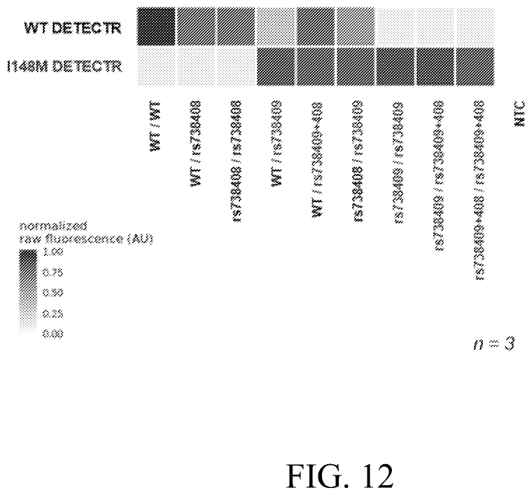

This example describes detection of at-risk PNPLA3 alleles in heterozygous samples. In a first assay, samples representing nine different homozygous and heterozygous genotypes with respect to PNPLA3 were tested using the pooled gRNAs identified and selected in EXAMPLE 10. FIG. 12 shows the results of a DETECTR assay to detect different homozygous or heterozygous combinations of PNPLA3 alleles. Samples were detected with pooled gRNAs designed to detect the wild type or non-risk alleles (“WT DETECTR”) or at-risk allele independent of the presence or absence of the non-risk allele (1148M DETECTR″). Samples heterozygous for the wild type or non-risk alleles and the at-risk allele were detected by both gRNA pools. NTC denotes a negative control lacking a target nucleic acid. These results show that the pooled gRNAs were functional to distinguish homozygous and heterozygous samples containing different combinations of at-risk, non-risk, and wild type alleles.

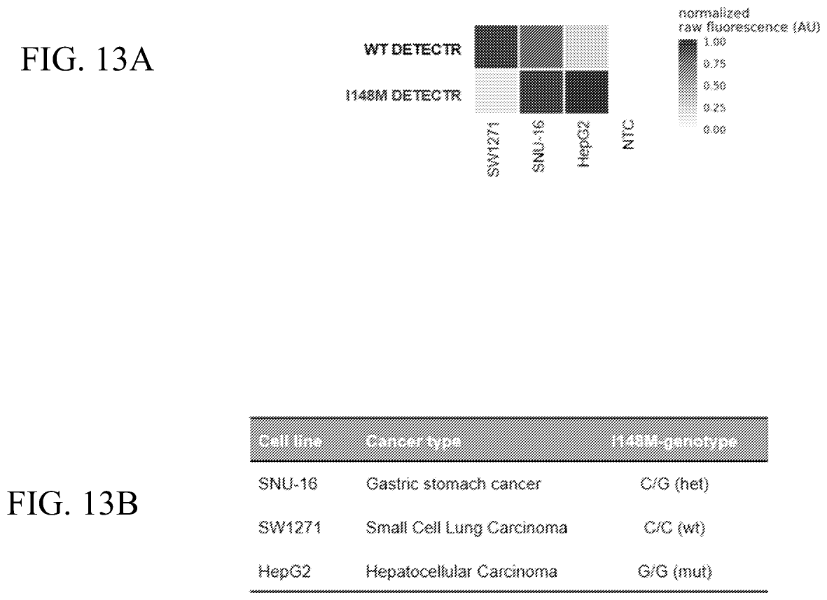

In a second assay, a DETECTR reaction was performed on cells from validated cell lines with different PNPLA3 genotypes. Samples were detected using the pooled gRNAs. FIG. 13A shows the results of a DETECTR assay to detect different PNPLA3 alleles in validated cell lines. Samples were detected with pooled gRNAs designed to detect the wild type or non-risk alleles (“WT DETECTR”) or at-risk allele independent of the presence or absence of the non-risk allele (1148M DETECTR″). SW1271 cells were heterozygous for the wild type allele, SNU-16 cells were heterozygous for wild type and at-risk alleles, and HepG2 cells were homozygous for the at-risk allele. NTC denotes a negative control lacking a target nucleic acid. The genotype of each cell line is provided in FIG. 13B. FIG. 13B shows the genotypes of the cell lines used in the assay shown in FIG. 13A. SW1271 cells are heterozygous for the wild type allele (“wt”), SNU-16 cells are heterozygous for wild type and at-risk alleles (“het”), and HepG2 cells are homozygous for the at-risk allele (“mut”). These results show that the DETECTR reaction with pooled gRNAs was functional to distinguish SNP phenotypes in homozygous and heterozygous cell samples.

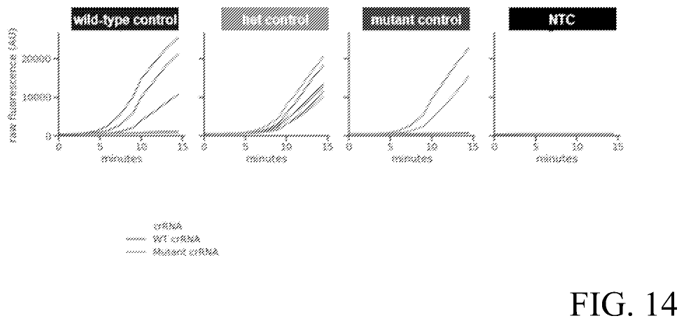

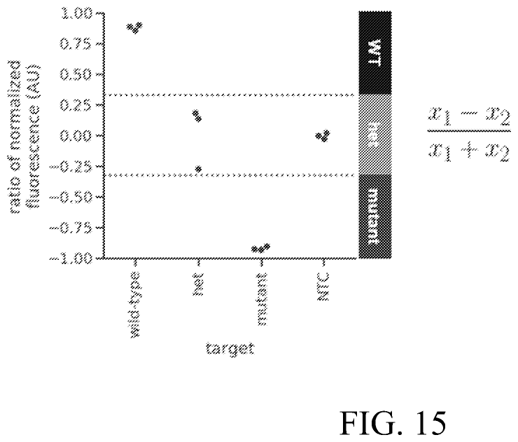

Samples containing synthetic control nucleic acids were assayed to determine a baseline fluorescence for each PNPLA3 genotype. FIG. 14 shows the results of a DETECTR assay measuring synthetic control samples for different genetic combinations of PNPLA3 alleles. Samples containing wild type synthetic control DNA (“wild-type control”), both wild type and at-risk allele synthetic control DNA (“het control”), at-risk allele synthetic control DNA (“mutant control”), or no target (“NTC”) were detected using gRNA directed to either the wild type sequence (“WT crRNA”) or the at-risk allele (“Mutant crRNA”). The resulting data was analyzed to determine threshold fluorescence ratios differentiate wild type, mutant, and heterozygous phenotypes. FIG. 15 shows the results of a DETECTR assay to detect the presence or absence of an at-risk PNPLA3 allele. Samples were either homozygous for the wild type allele (“wild-type”), heterozygous for the wild type allele and the at-risk allele (“het”), homozygous for the at-risk allele (“mutant”), or contained no target (“NTC”). Threshold fluorescence intensity levels, indicated by dashed horizontal lines, were set to distinguish between wild type, heterozygous, and at-risk sequences. Together, these results show that DETECTR can be used to differentiate samples that are homozygous or heterozygous for single nucleotide polymorphisms.

### Example 14

### Detection of at-Risk PNPLA3 Alleles in Heterozygous Samples from Human Subjects

This example describes detection of at-risk PNPLA2 alleles in heterozygous samples from human subjects. The DETECTR assays described in EXAMPLE 13 were used to assay samples collected from human subjects to determine their genotype with respect to an at-risk mutation in PNPLA3. Genotype was determined based on the threshold fluorescence ratios determined from the synthetic control assays performed in EXAMPLE 13. Sample genotypes were verified using a Taqman qPCR assay, which was the current gold standard genotyping assay in the field.

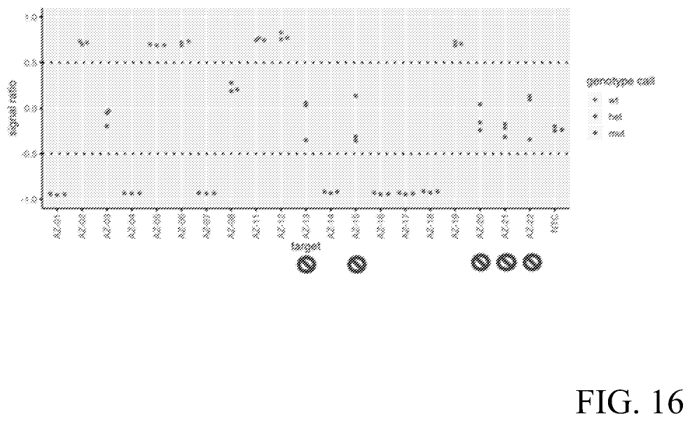

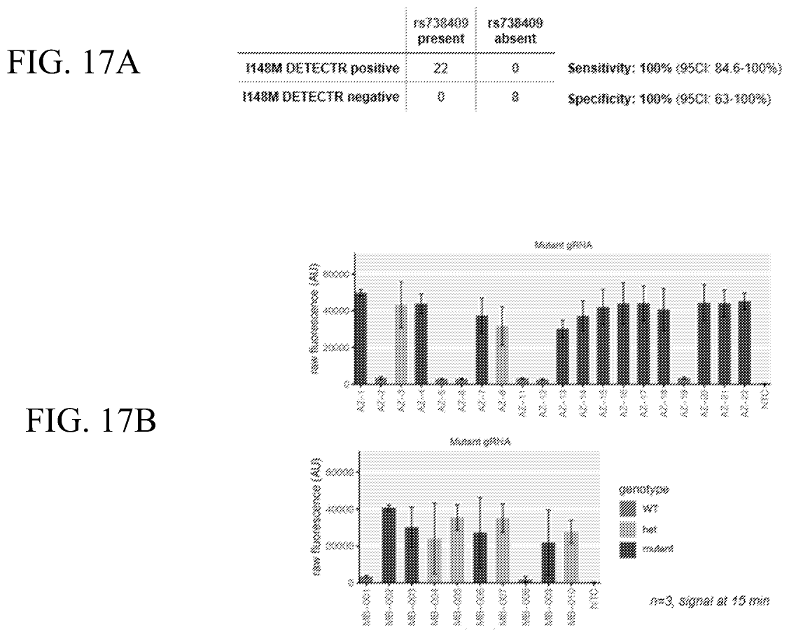

22 human samples were assayed using a DETECTR assay performed using a CasY3 programmable nuclease. FIG. 16 shows the results of a DETECTR assay to determine PNPLA3 genotype of 22 samples (AZ-01 through AZ-22). Samples were classified as homozygous wild type, heterozygous, or homozygous at-risk mutant based on threshold levels (horizontal dotted lines) of the fluorescence signal ratio. A sample without DNA (“NTC”) was used as a negative control. The DETECTR assay was performed using CasY3 (SEQ ID NO: 3). The DETECTR classification was compared to the genotype call, homozygous wild type (“wt”), heterozygous (“het”), or homozygous at-risk mutant (“mut”), determined by Taqman qPCR analysis (colored dots). The DETECTR classification had 100% concordance with the qPCR classification. In each case, the genotype classification from the DETECTR assay matched the genotype determined by qPCR analysis. FIG. 17A shows a comparison of DETECTR assays detecting the presence or absence of a PNPLA3 mutation (I148M DETECTR positive or I148M DETECTR negative, respectively) to the at-risk genotype encoding for the wild type sequence (rs738409 absent) or the mutant sequence (rs738409 present). The DETECTR assay showed 100% sensitivity (no false negatives), with a 90% confidence interval of 84.6% to 100%, and 100% specificity (no false positives), with a 95% confidence interval of 63% to 100%.

For further validation, the DETECTR assay was performed on additional human samples having various PNPLA3 genotypes. FIG. 17B shows the raw fluorescence of the DETECTR assay to determine PNPLA3 genotype of 22 samples (AZ-01 through AZ-22), shown in FIG. 16, and 10 additional samples (MB-001 through MB-010). Samples without DNA (“NTC”) were used as negative controls. The DETECTR assay was performed using CasY3 (SEQ ID NO: 3). The genotype call, homozygous wild type (“wt”), heterozygous (“het”), or homozygous at-risk mutant (“mut”), determined by Taqman qPCR analysis (bar shading).

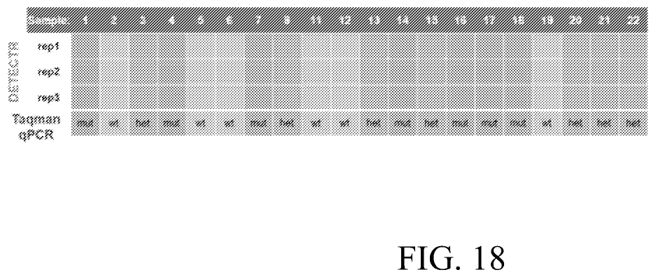

The results from the DETECTR assays to detect the presence or absence of an at-risk PNPLA3 allele in blinded samples are summarized in FIG. 18. Shading of the row denoted “Taqman qPCR” represents the genotype call, homozygous wild type (“wt”), heterozygous (“het”), or homozygous at-risk mutant (“mut”), determined by Taqman qPCR analysis. Shading of the rows denoted repeats 1 through 3 (rep1 through rep3) represents the genotype classification determined by DETECTR assay using a CasY3 (SEQ ID NO: 3). The results matched for DETECTR assays showed 100% agreement with the Taqman qPCR assay.

FIG. 19 shows the results of a DETECTR assay testing nucleotide spacer lengths. Different GFP target sites (T1-T9, from left to right and top to bottom, T3 corresponds to SEQ ID NO: 42) were targeted by CasY3 (SEQ ID NO: 3) and various crRNAs. crRNAs contained either a 7 nucleotide or 8-nucleotide repeat and either a 17 nucleotide or 18 nucleotide spacer. crRNAs are denoted at the top of each plot in parentheses as: (repeat length-spacer length). FIG. 20 shows the results of a DETECTR assay to test the temperature sensitivity CasY programmable nucleases. DETECTR assays were performed in the presence of 125 nM intermediary RNA (R1083, SEQ ID NO: 33), 125 nM crRNA (R801, SEQ ID NO: 37), 100 nM T8 reporter (SEQ ID NO: 21), and 20 nM GFP-T3 target (SEQ ID NO: 42). The programmable nuclease was incubated with the crRNA and the intermediary RNA at the indicated temperature and then moved to ice before performing the DETECTR assay.

### Example 15

### PAM Screening for CasY Proteins

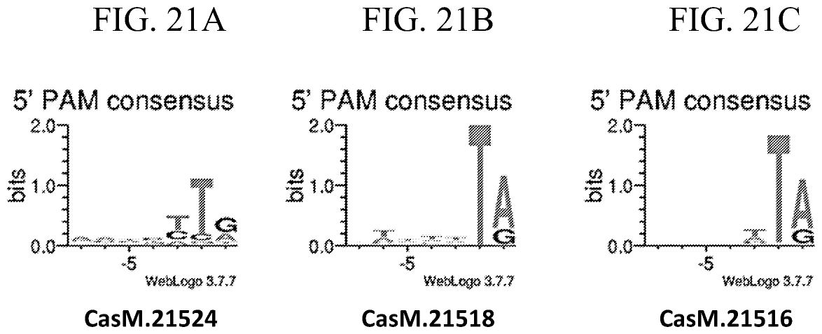

Cas proteins of SEQ ID NOs: 118-123 (TABLE 1) were screened by in vitro enrichment (IVE) for cis cleavage to determine recognized PAMs, using corresponding sgRNA as shown in TABLE 8. Briefly, Cas proteins were complexed with corresponding sgRNAs for 15 minutes at 37° C. The RNA protein (RNP) complexes were at 10× concentration (1 μl of 10× Cutsmart buffer, 1 μl of protein, 500 nM for sgRNA). After complexing 1:10 dilution was done with all the complexes. The undiluted and diluted complexes were added to the IVE reaction mix. PAM screening reactions used 10 μl of RNP in 100 μl reactions with 1,000 ng of a 5′ PAM library in 1× Cutsmart buffer and were carried out for 15 minutes at 25° C., 45 minutes at 37° C. and 15 minutes at 45° C. Reactions were terminated with 1 μl of proteinase K and 5 μl of 500 mM EDTA for 30 minutes at 37° C. Next generation sequencing was performed on cut sequences to identify enriched PAMs. As shown in TABLE 9, cis cleavage was observed with RNP complexes comprising CasM.21524, CasM.21518 or CasM.21516 proteins and corresponding sgRNAs. FIGS. 21A, 21B, and 21C illustrate the composition of the sequences derived from libraries digested with RNP complexes comprising CasM.21524, CasM.21518, and CasM.21516 proteins. FIG. 21A illustrates PAM preferences for a CasM.21524 protein. Frequency of nucleotides at each PAM position was independently calculated using a position frequency matrix (PFM) and plotted as a WebLogo. FIG. 21B illustrates PAM preferences for a CasM.21518 protein. Frequency of nucleotides at each PAM position was independently calculated using a position frequency matrix (PFM) and plotted as a WebLogo. FIG. 21C illustrates PAM preferences for a CasM.21516 protein. Frequency of nucleotides at each PAM position was independently calculated using a position frequency matrix (PFM) and plotted as a WebLogo. Examination of the PFM derived WebLogos (FIGS. 21A, 21B, and 21C) revealed the presence of enriched 5′ PAM consensus sequences for CasM.21524, CasM.21518, and CasM.21516 were NNNNNTR, where R is a purine and N is any nucleotide.

TABLE 8
Exemplary Nucleotide Sequence of sgRNA

| | SEQ ID | |
|---|---|---|
| sgRNA | NO: | Sequence |
| R4997 | 124 | CUUCGCCUCGUCCUCGGAGCAAGCUCC |
| | | UGUGGGCGAGCCUUUGAAAAGGCUAUU |
| | | AAAUACUCGUAUUG |
| R5001 | 125 | UUUUCCCCAACUGAAAGGUUGGAUGCC |
| | | UUUCAAAAGGCUAUUAAAUACUCGUAU |
| | | UG |
| R4999 | 126 | AUGUUCCAGGUUCUUUCGGGAGCCUUG |
| | | GCCUUUAUGAAGGCUAUUAAAUACUCG |
| | | UAUUG |
| R4993 | 127 | GCCAGUUUGGGAAACCUGGGUCUUUAU |
| | | UUUUAAAGACACAGGAAUUCCCGCGUC |
| | | UUUGUAAAGACUAUUAAAUACUCGUAU |
| | | UG |
| R4995 | 128 | CUUUUCCUUCCCCAAAAGGGAAGUUGC |
| | | CUUUUAAAAGGCUAUUAAAUACUCGUA |
| | | UUG |

TABLE 9
Exemplary cis-Cleavage Activity of Compositions
Comprising CasY and Corresponding sgRNA

| | | | cis-cleavage |
|---|---|---|---|
| | | | PAM (NNNNNNN) |
| | | cis- | ‘.’ indicates |
| Cas Y | | cleavage | location of spacer |
| Protein | sgRNA | (y/n) | relative to the PAM |
| CasM.21524 | R4997 | Y | NNNNNTR |
| | | | (SEQ ID NO. 129) |
| CasM.21518 | R5001 | Y | NNNNNTR |
| | | | (SEQ ID NO. 129) |
| CasM.21520 | R4999 | N | — |
| CasM.21522 | R4993 | N | — |
| CasM.21516 | R4995 | Y | NNNNNTR |
| | | | (SEQ ID NO. 129) |
| CasM.21466 | R4993 | N | — |

### Example 16

### CasY Proteins Provide Trans-Cleavage Activity

CasY proteins were tested for trans cleavage. Briefly, partially purified (nickel-NTA purified) CasY proteins were incubated with corresponding sgRNAs in low salt buffer at room temperature for 20 minutes, followed by addition of target nucleic acid at a final concentration of 10 nM. Low salt buffer is 20 mM Tricine, 15 mM MgCl₂, 0.2 mg/ml BSA, 1 mM TCEP (pH 9) at 37° C. The sgRNA sequences are provided in TABLE 8. As TABLE 10, the target nucleic acid was either (i) dsDNA containing the “51” protospacer target downstream of a 7N PAM, where N is any nucleotide, (ii) dsDNA containing the “51” protospacer target downstream of a TTTG PAM or (iii) single stranded DNA (ss 51) containing the “51” protospacer target downstream of a TTTG PAM. Trans cleavage activity was detected by fluorescence signal upon cleavage of a 12-T fluorophore-quencher reporter in a DETECTR reaction. A 12-T fluorophore—quencher-labeled ssDNA molecule that is cleaved upon CasY trans-activity generated a fluorescence readout. Trans cleavage activity signal was reported as a maximum rate of fluorescence accumulation of the experimental condition (containing target, +target) over that for the control (no target, −target). High fluorescence background was observed with the negative control (−target) compared to that with the counterpart target sample (+target), especially at higher protein concentrations. To resolve this issue, dilutions of the protein were performed, and the assay repeated at 1%, 0.1% or 0.01% dilutions of the original protein concentration. Trans cleavages were observed with RNP complexes comprising CasM21524 and CasM21520 proteins and corresponding sgRNAs (TABLE 10).

TABLE 10
Exemplary trans-Cleavage Activity of Compositions
Comprising CasY and Corresponding sgRNA

| | | trans-cleavage | trans cleavage |
|---|---|---|---|
| | | (y/n; active if | activity |
| | | trans cleavage | signal (max |
| CasY | | activity | rate exp/max |
| Protein | sgRNA | signal >1.5) | rate neg ctrl) |
| CasM.21524 | R4997 | Y | 2.9 |
| CasM.21518 | R5001 | N | — |
| CasM.21520 | R4999 | Y | 2.1 |
| CasM.21522 | R4993 | N | — |
| CasM.21516 | R4995 | N | — |
| CasM.21466 | R4993 | N | — |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A composition comprising
a programmable nuclease or a nucleic acid encoding the programmable nuclease; and
an engineered guide RNA comprising a crRNA or a nucleic acid encoding the crRNA,
wherein a repeat of the crRNA is no more than 24 bases in length.

2. The composition of claim 1, wherein a sequence of the repeat comprises 5′-AAGGC-3′.

3. The composition of any one of claims 1-2, wherein the engineered guide RNA comprises an intermediary RNA.

4. The composition of claim 3, wherein the intermediary RNA comprises a repeat hybridization region no more than 7 bases complementary to a sequence of the crRNA.

5. The composition of any one of claims 3-4, wherein the intermediary RNA comprises a repeat hybridization region no more than 5 bases complementary to a sequence of the crRNA.

6. The composition of any one of claims 4-5, wherein the repeat hybridization region is exposed in a bubble within a stem of a hairpin stem-loop structure of the intermediary RNA.

7. The composition of any one of claims 1-6, wherein the crRNA comprises a repeat and a spacer.

8. The composition of any one of claims 1-7, further comprising a target nucleic acid.

9. The composition of any one of claims 7-8, wherein the spacer is complementary to a target sequence of the target nucleic acid.

10. The composition of any one of claims 8-9, wherein the target nucleic acid is DNA.

11. The composition of claim 10, wherein the DNA is single stranded DNA.

12. The composition of claim 10, wherein the DNA is double stranded DNA.

13. The composition of any one of claims 7-12, wherein the spacer comprises 15 to 20 bases.

14. The composition of claim 13, wherein the spacer comprises 17 to 19 bases.

15. The composition of any one of claims 13-14, wherein the spacer comprises 17 bases.

16. The composition of any one of claims 7-15, wherein the repeat comprises 5 to 20 bases.

17. The composition of claim 16, wherein the repeat comprises 7-8 bases.

18. The composition of any one of claims 16-17, wherein the repeat comprises 5 bases.

19. The composition of any one of claims 7-18, wherein the repeat further comprises A, U, or C 5′ of the 5′-AAGGC-3′.

20. The composition of claim 19, wherein the repeat comprises A or U 5′ of the 5′-AAGGC-3′.

21. The composition of any one of claims 1-20, wherein the intermediary RNA comprises an RNA hairpin of from 20 to 56 bases.

22. The composition of any one of claims 1-21, wherein the intermediary RNA comprises an RNA hairpin of 21 bases.

23. The composition of any one of claims 1-22, wherein the intermediary RNA comprises an RNA hairpin of 25 bases.

24. The composition of any one of claims 1-23, wherein the intermediary RNA comprises an RNA hairpin of 56 bases.

25. The composition of any one of claims 4-24, wherein the repeat hybridization region is positioned at a 3′ end of the RNA hairpin.

26. The composition of any one of claims 4-25, wherein a sequence of the repeat hybridization region comprises 5′ GCCUU 3′.

27. The composition of any one of claims 21-26, wherein the intermediary RNA comprises a sequence 5′ of the RNA hairpin that hybridizes to a sequence 3′ of the repeat hybridization region.

28. The composition of any one of claims 4-27, wherein the intermediary RNA comprises from 50 to 105 bases.

29. The composition of claim 28, wherein the intermediary RNA comprises 50 bases.

30. The composition of any one of claims 4-29, wherein the intermediary RNA comprises a 5′AU sequence adjacent and 5′ of the 5 bases complementary to the sequence of the crRNA.

31. The composition of any one of claims 8-30, wherein the target nucleic acid comprises a protospacer adjacent motif (PAM) of TR or TTR, wherein R is A or G.

32. The composition of any one of claims 1-31, wherein the engineered guide RNA is a discrete engineered guide RNA system.

33. The composition of any one of claims 1-31, wherein the engineered guide RNA is a composite engineered guide RNA.

34. The composition of claim 33, wherein the crRNA and the intermediary RNA of the composite engineered guide RNA are linked.

35. The composition of claim 34, wherein the crRNA is adjacent and 3′ of the intermediary RNA.

36. The composition of any one of claims 33-35, wherein the composite engineered guide RNA comprises fewer than 100 bases.

37. The composition of any one of claims 33-36, wherein the composite engineered guide RNA comprises 50 to 100 bases.

38. The composition of any one of claims 33-37, wherein the composite engineered guide RNA comprises 63 bases.

39. The composition of any one of claims 33-38, wherein the crRNA is positioned at a 3′ end of the repeat hybridization region of the intermediary RNA.

40. The composition of any one of claims 33-39, wherein the composite engineered guide RNA comprises a tetraloop between the 5′-AAGGC-3′ sequence of the crRNA and the repeat hybridization region of the intermediary RNA.

41. The composition of claim 40, wherein the tetraloop comprises a U, G, A, or any combination thereof.

42. The composition of any one of claims 40-41, wherein the tetraloop is 5′-XGAU-3′, where X is any base.

43. The composition of claim 42, wherein the tetraloop is 5′-UGAU-3′.

44. The composition of any one of claims 1-43, wherein the programmable nuclease is a Cas12 protein.

45. The composition of claim 44, wherein the Cas12 protein is CasY.

46. The composition of claim 45, wherein the CasY has at least 60%, at least 70%, at least 80%, at least 85%, at least 90%, at least 92%, at least 95%, at least 97%, at least 99%, or 100% sequence identity with any one of SEQ ID NOs: 1-10.

47. The composition of claim 45, wherein the CasY has at least 60%, at least 70%, at least 80%, at least 85%, at least 90%, at least 92%, at least 95%, at least 97%, at least 99%, or 100% sequence identity with any one of SEQ ID NOs: 118-123.

48. The composition of claim 45, wherein the CasY has at least 60%, at least 70%, at least 80%, at least 85%, at least 90%, at least 92%, at least 95%, at least 97%, at least 99%, or 100% sequence identity with any one of SEQ ID-NOs: 1-10 and 118-123.

49. The composition of any one of claims 1-48, wherein the composition is at a temperature of up to and including 30° C.

50. The composition of any one of claims 1-49, wherein the composition is at a temperature of up to and including 37° C.

51. The composition of any one of claims 1-50, wherein the composition is at a pH of from 7 to 9.

52. The composition of claim 51, wherein the composition is at a pH of from 7.1 to 9.

53. The composition of any one of claims 51-52, wherein the composition is at a pH of from 8.5 to 9.

54. The composition of any ne of claims 51-53, wherein the composition is at a pH of about 8.5.

55. The composition of any one of claims 51-53, wherein the composition is at a pH of about 8.8.

56. A method of modifying a target nucleic acid, the method comprising contacting the composition of any one of claims 1-55 to the target nucleic acid.

57. The method of claim 56, wherein the modifying comprises introducing a double stranded break in the target nucleic acid.

58. The method of claim 56, wherein the programmable nuclease comprises an enzymatically dead programmable nuclease.

59. The method of claim 56, wherein the modifying comprises transcriptional activation.

60. The method of claim 58, wherein the enzymatically dead programmable nuclease is fused to a transcriptional activator.

61. The method of claim 60, wherein the transcriptional activator comprises VP16, VP64, VP48, VP160, a p65 subdomain, an EDLL activation domain, a TAL activation domain, SET1A, SET1B, MLL1 to 5, ASH1, SYMD2, NSD1, JHDM2a/b, UTX, JMJD3, GCN5, PCAF, CBP, p300, TAF1, TIP60/PLIP, MOZ/MYST3, MORF/MYST4, SRC1, ACTR, P160, CLOCK, Ten-Eleven Translocation (TET) dioxygenase 1 (TET1CD), TET1, DME, DML1, DML2, or ROS1.

62. The method of claim 56, wherein the modifying comprises transcriptional repression.

63. The method of claims 58 and 62, wherein the enzymatically dead programmable nuclease is fused to a transcriptional repressor.

64. The method of claim 63, wherein the transcriptional repressor comprises a Krüppel associated box (KRAB or SKD); a KOX1 repression domain; a Mad mSIN3 interaction domain (SID); an ERF repressor domain (ERD), a SRDX repression domain, Pr-SET7/8, SUV4-20H1, RIZ1, JMJD2A/JHDM3A, JMJD2B, JMJD2C/GASC1, JMJD2D, JARID1A/RBP2, JARID1B/PLU-1, JARID1C/SMCX, JARID1D/SMCY, HDAC1, HDAC2, HDAC3, HDAC8, HDAC4, HDAC5, HDAC7, HDAC9, SIRT1, SIRT2, HDAC11, HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2, Lamin A, or Lamin B.

65. The method of any one of claims 56-64, wherein the target nucleic acid is a target DNA.

66. The method of claim 65, wherein the target DNA is from an animal.

67. The method of claim 65, wherein the target DNA is from a plant.

68. The method of any one of claims 66-67, wherein the target DNA is target chromosomal DNA.

69. The method of any one of claims 56-68, further comprising administering the composition to a cell.

70. The method of claim 69, further comprising inducing production of a biologic by the cell.

71. The method of any one of claims 56-70, further comprising administering the composition to a subject in need thereof.

72. The method of claim 71, wherein the subject is a human.

73. A method of assaying for a target nucleic acid in a sample from a subject, the method comprising:
contacting the sample to:
the composition of any one of claims 1-55;
a detector nucleic acid; and
assaying for a signal produced by cleavage of the detector nucleic acid.

74. The method of claim 73, wherein the target nucleic acid is DNA.

75. The method of claim 73, wherein the target nucleic acid is RNA.

76. The method of claim 75, the method further comprising reverse transcribing the RNA prior to the contacting.

77. The method of any one of claims 73-76, the method further comprising amplifying the target nucleic acid prior to the contacting.

78. The method of any one of claims 73-77, wherein the target nucleic acid is viral DNA or bacterial DNA.

79. The method of claim 78, wherein the viral DNA is from papovavirus, human papillomavirus (HPV), hepadnavirus, Hepatitis B Virus (HBV), herpesvirus, varicella zoster virus (VZV), epstein-barr virus (EBV), kaposi's sarcoma-associated herpesvirus, adenovirus, poxvirus, or parvovirus, an influenza virus, a respiratory syncytial virus, or a coronavirus.

80. The method of any one of claims 73-77, wherein the target nucleic acid comprises a single nucleotide polymorphism.

81. The method of claim 80, wherein the signal is produced in the presence of the target nucleic acid comprising a first variant at the single nucleotide polymorphism, and wherein the signal is higher in the presence of the target nucleic acid comprising the first variant at the single nucleotide polymorphism than in the presence of the target nucleic acid comprising a second variant at the single nucleotide polymorphism.

82. The method of claim 80, further comprising distinguishing a first variant and a second variant of the single nucleotide polymorphism.

83. The method of any one of claims 73-82, further comprising determining a homozygous or heterozygous genotype of the sample for a first variant and a second variant of the target nucleic acid.

84. The method of claim 83, wherein the sample is heterozygous for a first variant and a second variant of the target nucleic acid.
